(19) United States
(12) Patent Application Publication
(10) Pub. No.: US 2023/0374541 A1
(43) Pub. Date: Nov. 23, 2023

(54) RECOMBINANT ADENO-ASSOCIATED VIRUSES FOR CNS OR MUSCLE DELIVERY

(71) Applicant: REGENXBIO INC., Rockville, MD (US)

(72) Inventors: Olivier Danos, Princeton, NJ (US); Samantha Yost, Rockville, MD (US); Andrew Mercer, Poolesville, MD (US); Ye Liu, Clarksville, MD (US); Joseph Bruder, Gaithersburg, MD (US); Subha Karumuthil-Melethil, Germantown, MD (US); Elad Firnberg, Baltimore, MD (US); Randolph Qian, Gaithersburg, MD (US); April R. Tepe, Laurel, MD (US); Jennifer M. Egley, Gaithersburg, MD (US)

(21) Appl. No.: 18/030,683

(22) PCT Filed: Oct. 7, 2021
(86) PCT No.: PCT/US2021/054058

§ 371 Date: Apr. 6, 2023

Related U.S. Application Data
(60) Provisional application No. 63/088,988, filed on Oct. 7, 2020.

(51) Int. Cl. C12N 15/86; A61K 48/00

(52) U.S. Cl. CPC C12N 15/86; A61K 48/0058; C12N 2750/14122; C12N 2750/14143

(57) ABSTRACT

The present invention relates to recombinant adeno-associated viruses (rAAVs) having capsid proteins engineered to include amino acid sequences and/or amino acid substitutions that confer and/or enhance desired properties, particularly increased transduction in CNS or muscle cells relative to a rAAV having a reference capsid.

### 1. FIELD OF THE INVENTION

The present invention relates to recombinant adeno-associated viruses (rAAVs) having capsid proteins with one or more amino acid substitutions and/or peptide insertions that confer and/or enhance desired properties, including tissue tropisms. In particular, the invention provides engineered capsid proteins comprising one or more amino acid substitutions or peptide insertions that enhance the tropism of an AAV serotype for one or more tissue types as well as capsids that are not engineered but are found to confer muscle or CNS tropisms on rAAVs. Particularly, the one or more amino acid substitutions and/or insertions in the AAV capsid improve transduction, genome integration and/or transgene expression in heart and/or muscle tissue or the central nervous system while reducing tropism for the liver and/or the dorsal root ganglion and/or peripheral nerve cells. rAAVs having the capsid proteins disclosed herein are useful for delivering a transgene encoding a therapeutic protein for treatment of CNS or muscle disease.

### 2. BACKGROUND

The use of adeno-associated viruses (AAV) as gene delivery vectors is a promising avenue for the treatment of many unmet patient needs. Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Without a helper virus, AAV establishes a latent infection. An AAV genome generally has a Rep gene and a Cap gene, flanked by inverted terminal repeats (ITRs), which serve as replication and packaging signals for vector production. The capsid proteins form capsids that carry genome DNA and can determine tissue tropism to deliver DNA into target cells.

Due to low pathogenicity and the promise of long-term, targeted gene expression, recombinant AAVs (rAAVs) have been used as gene transfer vectors, in which therapeutic sequences are packaged into various capsids. Such vectors have been used in preclinical gene therapy studies and over twenty gene therapy products are currently in clinical development. Recombinant AAVs, such as recombinant AAV9 particles, have demonstrated desirable neurotropic properties and clinical trials using recombinant AAV9 for treatment of CNS disease are underway. Delivery to muscle and/or heart tissue is also desirable. Reduction of transduction of liver and/or dorsal root ganglion cells may also be desirable to reduce toxicity. However, attempts to enhance the neurotropic or muscle/heart tropic properties of rAAVs in human subjects have met with limited success.

There remains a need for rAAV vectors with enhanced neurotropic or with tropism for muscle and/or heart properties for use, e.g., in treating disorders associated with the central nervous system or where expression in the heart and/or muscle are desirable, with minimal transduction in liver and/or dorsal root ganglion cells and/or peripheral nerve cells to minimize adverse effects. There also is a need for rAAV vectors with enhanced tissue-specific targeting and/or enhanced tissue-specific transduction to deliver therapies.

### 3. SUMMARY OF THE INVENTION

Provided are recombinant adeno-associated viruses (rAAVs) having capsid proteins engineered to have one or more amino acid substitutions and/or peptide insertion that enhance tissue targeting, transduction and/or integration of the rAAV genome in CNS and/or muscle tissue relative to a reference capsid, for example, the parent capsid or an AAV8 or AAV9 capsid, while having reduced biodistribution in certain tissues, such as liver and dorsal root ganglion cells, relative to the distribution in CNS and/or muscle and/or relative to the parent capsid or a reference capsid, such as AAV8 or AAV9 capsid, to reduce toxicity. Biodistribution studies in mice and non-human primates permit assessment of relative transduction and transgene transcription and expression in tissue types of capsids, including engineered capsids (see, Examples 13-18, infra). Accordingly, provided herein are rAAVs with enhanced or increased biodistribution, including transduction, genome integration, transgene transcription and expression, in CNS tissues (including frontal cortex, hippocampus, cerebellum, midbrain) relative to a reference capsid (for example the unengineered, parental capsid that has been modified or AAV8 or AAV9), with reduced distribution, including transduction, genome integration, transgene transcription and expression in the liver and/or dorsal root ganglion cells (cervical, thoracic, and/or lumbar) compared to the biodistribution in CNS tissue and/or relative to an AAV with a reference capsid, such as the parental capsid or AAV8 or AAV9. Such rAAVs may be useful to deliver therapeutic proteins for the treatment of CNS disease. In addition, provided herein are rAAVs with enhanced or increased biodistribution, including transduction, genome integration, transgene transcription and expression, in skeletal muscle and/or cardiac muscle tissues relative to a reference capsid (for example the unengineered, parental capsid or AAV8 or AAV9), with reduced distribution, including transduction, genome integration, transgene transcription and expression in the liver and/or dorsal root ganglion cells (cervical, thoracic, and/or lumbar) compared to the biodistribution in muscle tissue and/or relative to an AAV with a reference capsid, such as the parental capsid or AAV8 or AAV9. Such rAAVs may be useful to deliver therapeutic proteins for the treatment of muscle disease.

In particular, provided are AAV9 capsid proteins or AAV8 capsid proteins (SEQ ID NO:67 or 66, respectively, and as numbered in FIG. 7) or having one or more amino acid substitutions (including, 2, 3 or 4 amino acid substitutions) that preferentially (in particular, at a greater level than rAAVs with the wild type AAV8 or AAV9 capsid) transduce (target) cells of the CNS, and, in certain embodiments, do not target or transduce, or have reduced transduction compared to rAAVs with wild type AAV8 or AAV9 capsids, the liver and/or the dorsal root ganglion and/or peripheral nerve cells. In other embodiments, provided are AAV9 capsid proteins or AAV8 capsid proteins (SEQ ID NO:67 or 66, respectively, and as numbered in FIG. 7) having one or more amino acid substitutions (including, 2, 3 or 4 amino acid substitutions) that preferentially (in particular, at a greater level than rAAVs with the wild type AAV9 capsid) transduce (target) cells of the heart and/or skeletal muscle, and, in certain embodiments, do not target or transduce, or have reduced transduction compared to rAAVs with wild type AAV8 or AAV9 capsids, the liver and/or the dorsal root ganglion and/or peripheral nerve cells. Such amino acid modifications include S263F/S269T/A273T of AAV9, and corresponding substitutions in other AAV type capsids (for example according to the alignment in FIG. 7), or W530R, Q474A, N272A, or G266A of AAV9, and corresponding substitutions in other AAV type capsids or A269S of AAV8 and corresponding substitutions in other AAV capsids (for example, according to the alignment in FIG. 7). Also provided are capsids, particularly AAV9 capsids having a peptide TLAAPFK (SEQ ID NO:1) inserted between Q588 and A589 (herein PHP.hDYN) or alternatively between 5268 and 5269 or between 5454 and G455) or inserted in another AAV capsid at a corresponding position (see, e.g., FIG. 7). Or, alternatively, the capsid is an AAV9 PHP.eB capsid (which has the modifications A587D and Q588G and insertion of the peptide TLAVPFK (SEQ ID NO:20) between G588 and A589) and the peptide TILSRSTQTG (SEQ ID NO:15) between position 138 and 139, or the corresponding. Additional capsids have a Kidney1 peptide LPVAS inserted into the capsid, for example between S454 and G455 of AAV9, or alternatively between 5268 and 5269 or between Q588 and A589, or the corresponding position of a different capsid. In some embodiments, the capsids can comprise R697W substitution of AAV rh64R1. The capsids having these amino acid substitutions and insertions may further have substitutions of the NNN (asparagines) at 496 to 498 with AAA (alanines) of the AAV9 capsid or at positions 498 to 500 of the AAV8 capsid, or corresponding substitutions in other AAV type capsids. Engineered capsids include AAV8.BB.LD (A269S,498-NNN/AAA-500 substitutions in the amino acid sequence of AAV8, SEQ ID NO 66), AAV9.BB.LD (S263G/S269T/A273T, 496-NNN/AAA-498 substitutions in the amino acid sequence of AAV9, SEQ ID NO 67), AAV9.496-NNN/AAA-498 (SEQ ID NO: 31), AAV9.496-NNN/AAA-498.W503R (SEQ ID NO: 32), AAV9.W503R (SEQ ID NO: 33), or AAV9.Q474A (SEQ ID NO: 34). In other examples, the capsid can be AAV9.N272A.496-NNN-498 (SEQ ID NO:91) or AAV9.G266A.496-NNN-498 (SEQ ID NO: 92). In other embodiments, the capsid is not an engineered capsid, but is an AAVrh.10 capsid (SEQ ID NO: 69), an AAVrh.46 capsid (SEQ ID NO:93), an AAVrh.64.R1 capsid (SEQ ID NO: 90) or an AAVrh.73 capsid (SEQ ID NO: 88). In certain embodiments, transduction is measured by detection of transgene, such as GFP fluorescence.

The capsid protein to be engineered may be an AAV9 capsid protein but may also be any AAV capsid protein, such as AAV serotype 1 (SEQ ID NO:59); AAV serotype 2 (SEQ ID NO:60); AAV serotype 3 (SEQ ID NO:61), AAV serotype 3B, AAV serotype 4 (SEQ ID NO:62); AAV serotype 5 (SEQ ID NO:63); AAV serotype 6 (SEQ ID NO:64); 451-461 of AAV7 capsid (SEQ ID NO:65); 451-461 of AAV8 capsid (SEQ ID NO:66); AAV serotype 9 (SEQ ID NO:67); AAV serotype 9e (SEQ ID NO:68); AAV serotype rh10 (SEQ ID NO:69); AAV serotype rh20 (SEQ ID NO:70); and AAV serotype hu.37 (SEQ ID NO:71), AAV serotype rh39 (SEQ ID NO:73), and AAV serotype rh74 (SEQ ID NO:72 or SEQ ID NO:80), AAV serotype rh.34, AAV serotype hu.60, AAV serotype rh.21, AAV serotype rh.15, AAV serotype rh.24, AAV serotype hu.5, AAV serotype hu.10 (SEQ ID NO: 69), AAV serotype rh64R1 (SEQ ID NO:90), AAV serotype rh46 (SEQ ID NO:93), and AAV serotype rh73 (SEQ ID NO: 88) (see FIG. 7 for alignment of certain sequences) and Table 17 for sequences. In some embodiments, the capsids of these vectors are not engineered. For example, unmodified AAV serotype rh64R1 (SEQ ID NO:90) AAV serotype rh.10 ((SEQ ID NO: 69) AAV serotype rh46 (SEQ ID NO:93), and AAV serotype rh73 (SEQ ID NO: 88) can be used in the disclosed methods and compositions.

In certain embodiments, provided are rAAVs incorporating the engineered capsids described herein, including rAAVs with genomes comprising a transgene of therapeutic interest, including a transgene encoding a therapeutic protein for treatment of a muscle, heart or CNS disease. Packaging cells for producing the rAAVs described herein are provided. Method of treatment by delivery of, and pharmaceutical compositions comprising, the engineered rAAVs described herein are also provided. Also provided are methods of manufacturing the rAAVs with the engineered capsids described herein.

The invention is illustrated by way of examples infra describing the construction of rAAV9 capsids engineered with amino acid substitutions and assaying of tissue distribution when administered to non-human primates.

### 3.1. Embodiments

1. A recombinant AAV capsid protein comprising one or more amino acid substitutions relative to the wild type or unengineered capsid protein, in which the rAAV capsid protein is an AAV9 capsid protein (SEQ ID NO:67) with S263G/S269R/A273T substitutions, a G266A substitution, an N272A substitution, a W503R substitution, a Q474A substitution, 496-NNN/AAA-498 substitutions, has an insertion of the peptide TLAAPFK between Q588 and A589, 5268 and 5269, or 5454 and G455, or is an AAV8 capsid (SEQ ID NO:6) with an A269S substitution or 498-NNN/AAA-500 substitutions, or corresponding substitutions or peptide insertions in a capsid protein of another AAV type capsid.

2. The recombinant AAV capsid protein of embodiment 1 further comprising 498-NNN/AAA-500 amino acid substitutions for an AAV8 capsid protein (SEQ ID NO: 66) or 496-NNN/AAA-498 amino acid substitutions for an AAV9 capsid protein (SEQ ID NO:67), or corresponding substitutions in a capsid protein of another AAV type capsid.

3. The recombinant AAV capsid protein of embodiments 1 or 2 which is an AAV8.BBB.LD capsid (SEQ ID NO: 27), an AAV9.BBB.LD capsid (SEQ ID NO: 29), an AAV9.496-NNN/AAA-498 capsid (SEQ ID NO: 31), AAV9.496-NNN/AAA-498 capsid (SEQ ID NO: 32), AAV9.W503R capsid (SEQ ID NO: 33), AAV9.Q474A capsid (SEQ ID NO: 34), AAV9.N272A.496-NNN/AAA-498 capsid (SEQ ID NO: 91) or AAV9.N266A.496-NNN/AAA-498 capsid (SEQ ID NO: 92).

4. The recombinant AAV capsid protein of embodiments 1 to 3 in which the amino acid substitutions or insertions are in an AAV9 capsid, including an AABPHP.eB capsid, protein, or an AAV8 capsid.

5. The recombinant AAV capsid protein of embodiment 1 or 2 wherein the AAV type capsid is AAV rh.34, AAV4, AAV5, AAV hu.26, AAV rh.31, AAV hu.13, AAV hu.26, AAV hu.56, AAV hu.53, AAV7, AAV rh.10, AAV rh.64.R1, AAV rh.46 or AAV rh.73.

6. The recombinant AAV capsid protein of any of embodiments 1 to 5, which when incorporated into a rAAV vector, the rAAV vector has increased targeting, transduction or genome integration into CNS cells, relative to a rAAV vector incorporating the corresponding wild type capsid protein without the amino acid substitutions or peptide insertions.

7. The recombinant capsid protein of embodiment 6, which when incorporated into a rAAV vector, the rAAV vector has decreased targeting, transduction or genome integration into liver cells, relative to a rAAV vector incorporating the corresponding wild type capsid protein without the amino acid substitutions or peptide insertions.

8. The recombinant capsid protein of embodiment 6 or 7, which when incorporated into a rAAV vector, the rAAV vector has decreased targeting, transduction or genome integration into dorsal root ganglion cells, relative to a rAAV vector incorporating the corresponding capsid protein without the amino acid substitutions or peptide insertions.

9. The recombinant capsid protein of any of the embodiments 6 to 8, which when incorporated into a rAAV vector, the rAAV vector has decreased targeting, transduction or genome integration into peripheral nerve cells, relative to a rAAV vector incorporating the corresponding capsid protein without the amino acid substitutions or peptide insertions.

10. The recombinant AAV capsid protein of any of embodiments 1 to 5, which when incorporated into a rAAV vector, the rAAV vector has increased targeting, transduction or genome integration into skeletal and/or cardiac muscle cells, relative to a rAAV vector incorporating the corresponding capsid protein without the amino acid substitutions or peptide insertions.

11. The recombinant capsid protein of embodiment 10, which when incorporated into a rAAV vector, the rAAV vector has decreased targeting, transduction or genome integration into liver cells, relative to a rAAV vector incorporating the corresponding capsid protein without the amino acid substitutions or peptide insertions.

12. The recombinant capsid protein of embodiment 10 or 11, which when incorporated into a rAAV vector, the rAAV vector has decreased targeting, transduction or genome integration into CNS cells, relative to a rAAV vector incorporating the corresponding capsid protein without the amino acid substitutions or peptide insertion.

13. The recombinant capsid protein of any of embodiments 10 to 12, which when incorporated into a rAAV vector, the rAAV vector has decreased targeting, transduction or genome integration into dorsal root ganglion cells, relative to an rAAV vector incorporating the corresponding capsid protein without the amino acid substitutions

14. A nucleic acid comprising a nucleotide sequence encoding the rAAV capsid protein of any of embodiments 1 to 13, or encoding an amino acid sequence sharing at least 80% identity therewith and retaining the biological activity of the capsid.

15. The nucleic acid of embodiment 14 encoding the rAAV capsid protein of any of embodiments 1 to 13.

16. A packaging cell capable of expressing the nucleic acid of embodiment 14 or 15 to produce AAV vectors comprising the capsid protein encoded by said nucleotide sequence.

17 A rAAV vector comprising the capsid protein of any of embodiments 1 to 13.

18. The rAAV vector of embodiment 17 further comprising a transgene encoding a therapeutic protein operably linked to a regulatory sequence for expression in the muscle and/or CNS cells.

19. A pharmaceutical composition comprising the rAAV vector of embodiment 17 or 18 and a pharmaceutically acceptable carrier.

20. A method of delivering a transgene to a cell, said method comprising contacting said cell with the rAAV vector of embodiment 17 or 18, wherein said transgene is delivered to said cell.

21. The method of embodiment 20 in which the cell is a CNS cell, cardiac muscle cell or skeletal muscle cell.

22. A method of delivering a transgene to a target tissue of a subject in need thereof, said method comprising administering to said subject the rAAV vector of embodiment 17 or 18, wherein the transgene is delivered to said target tissue.

23. The method of embodiment 22 wherein the transgene is a muscle disease or heart disease therapeutic and said target tissue is cardiac muscle or skeletal muscle.

24. The method of embodiment 23, wherein the rAAV is administered systemically, including intravenously or intramuscularly.

25. The method of embodiment 22 wherein the transgene is a CNS disease therapeutic and said target tissue is CNS.

26. The method of embodiment 25 wherein the rAAV is administered intrathecally or intracerebroventricularly.

27. A pharmaceutical composition for use in delivering a transgene to a cell, said pharmaceutical composition comprising the rAAV vector of embodiment 17 or 18, wherein said transgene is delivered to said cell.

28. A pharmaceutical composition for use in delivering a transgene encoding a therapeutic protein to a target tissue of a subject in need thereof, said pharmaceutical composition comprising the rAAV vector of embodiment 17 or 18, wherein the transgene is delivered to said target tissue.

29. The pharmaceutical composition of embodiment 27 or 28 wherein said therapeutic protein is a muscle disease therapeutic or a heart disease therapeutic and said target tissue is cardiac muscle or skeletal muscle.

30. The pharmaceutical composition of embodiment 27 to 29 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction in cardiac muscle or skeletal muscle cells compared to a reference AAV capsid.

31. The pharmaceutical composition of embodiment 27 to 30 wherein the rAAV exhibits at least 50%, 60%, 70%, 80%, 90%, 95% or 99% less transduction in liver compared to the reference AAV capsid.

32. The pharmaceutical composition of embodiment 27 to 31 wherein the rAAV exhibits at least 50%, 60%, 70%, 80%, 90%, 95% or 99% less transduction in dorsal root ganglion cells compared to the reference AAV capsid.

33. The pharmaceutical composition of embodiment 27, 28 or 32 wherein said therapeutic protein is a CNS disease therapeutic and said target tissue is CNS.

34. The pharmaceutical composition of embodiment 27, 28, 32 or 33 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction in CNS cells compared to a reference AAV capsid.

35. The pharmaceutical composition of embodiment 27, 28, 33 to 34 wherein the rAAV exhibits at least 50%, 60%, 70%, 80%, 90%, 95% or 99% less transduction in liver compared to the reference AAV capsid.

36. The pharmaceutical composition of embodiment 27, 28, 32 to 35 wherein the rAAV exhibits at least 50%, 60%, 70%, 80%, 90%, 95% or 99% less transduction in dorsal root ganglion cells compared to the reference AAV capsid.

37. The pharmaceutical composition of embodiments 27 to 36, wherein the AAV reference capsid is AAV8 or AAV9.

38. A method of treating a CNS disorder in a subject in need thereof, said method comprising administering a therapeutically effective amount of pharmaceutical composition of any of embodiments 27, 28, 32 to 37.

39. A method of treating a muscle disorder in a subject in need thereof, said method comprising administering a therapeutically effective amount of the pharmaceutical composition of any of embodiments 27-31 and 37.

### 4. BRIEF DESCRIPTION OF THE FIGURES

FIG. 1 depicts sequence comparison of the capsid amino acid sequences including the VR-IV loop of the adeno-associated virus type 9 (AAV9 VR-IV) from residues L447 to R476, (with residues 451-459 bracketed) to corresponding to regions of other AAVs. Figure discloses SEQ ID NOS:49-54, 50, and 55-58, respectively, in order of appearance. The top sequence is the consensus sequence, SEQ ID NO:48.

FIG. 2 depicts a protein model of an AAV capsid structure, showing capsid variable regions VR-IV, VR-V and VR-VIII. The box highlights the loop region of VR-IV which provides surface-exposed amino acids as represented in the model.

FIG. 3 depicts high packaging efficiency (titer) in terms of genome copies per mL (GC/mL) of wild type AAV9 and eight (8) candidate modified rAAV9 vectors (1090, 1091, 1092, 1093, 1094, 1095, 1096, and 1097), where the candidate vectors each contain a FLAG insert immediately after different sites within AAV9s VR-IV, from residues 1451 to Q458, respectively. All vectors were packaged with luciferase transgene in 10 mL culture; error bars represent standard error of the mean.

FIG. 4 demonstrates surface exposure of 1 VR-IV loop FLAG inserts in each of eight (8) candidate modified rAAV9 vectors (1090, 1091, 1092, 1093, 1094, 1095, 1096, and 1097), confirmed by immunoprecipitation of packaged vectors by binding to anti-FLAG resin.

FIGS. 5A-5B depict transduction efficiency in Lec2 cells, transduced with capsid vectors carrying the luciferase gene (as a transgene), which were packaged into either wild type AAV9 (9-luc), or into each of eight (8) candidate modified (FLAG peptide inserted) rAAV9 vectors (1090, 1091, 1092, 1093, 1094, 1095, 1096, and 1097); transduction activity is expressed as percent luciferase activity, taking the activity of 9-luc as 100% (FIG. 5A), or as Relative Light Units (RLU) per microgram of protein (FIG. 5B).

FIGS. 6A-6E. FIG. 6A depicts a bar graph illustrating that insertions immediately after 5454 of AAV9 of varying peptide length and composition may affect production efficiencies of AAV particles in a packaging cell. Ten peptides of varying composition and length were inserted after S454 within AAV9 VR-IV. qPCR was performed on harvested supernatant of transfected suspension HEK293 cells five days post-transfection. The results depicted in the bar graph demonstrate that the nature of the insertions affects the ability of AAV particles to be produced and secreted by HEK293 cells, and indicated by overall yields (titer). (Error bars represent standard error of the mean length of peptide, which is noted on the Y-axis in parenthesis.) FIGS. 6B-6E depict fluorescence images of transduced cell cultures of the following cell lines: (6B) Lec2 cell line (6C) HT-22 cell line, (6D) hCMEC/D3 cell line, and (6E) C2C12 cell line. AAV9 wild type and S454 insertion homing peptide capsids containing GFP transgene were used to transduce the noted cell lines. P1 vector was not included in images due to extremely low transduction efficiency, and P8 vector was not included due to low titer. AAV9.S454.FLAG showed low transduction levels in every cell type tested.

FIG. 7 depicts alignment of AAVs 1-9e, 3B, rh10, rh20, rh39, rh73, rh74 version 1 and version 2, hu12, hu21, hu26, hu37, hu51 and hu53 capsid sequences with insertion sites for heterologous peptides after the initiation codon of VP2, and within or near variable region 1 (VR-D, variable region 4 (VR-IV), and variable region 8 (VR-VIII), all highlighted in grey; a particular insertion site within variable region eight (VR-VIII) of each capsid protein is shown by the symbol “#” (after amino acid residue 588 according to the amino acid numbering of AAV9).

FIG. 8 depicts copies of GFP (green fluorescent protein) transgene in mice brain cells, following administration of the AAV vectors: AAV9; AAV.PHP.eB, also referred to herein as AAV9e (AAV9 with the peptide TLAVPFK (SEQ ID NO:20) inserted between positions 588 and 589 and modifications A587D/A588G); AAV.hDyn (AAV9 with TLAAPFK (SEQ ID NO:1) between 588 and 589); AAV.PHP.S (AAV9 with the peptide QAVRTSL (SEQ ID NO:16) inserted between positions 588 and 589); and AAV.PHP.SH (AAV9 with the peptide QAVRTSH (SEQ ID NO:17) inserted between positions 588 and 589).

FIGS. 9A-9C depict the amino acid sequences for a recombinant AAV9 vector including a peptide insertion of TLAAPFK (SEQ ID NO:1) between Q588 and A589 (FIG. 9A), between 5268 and 5269 of VR-III (FIG. 9B), and between S454 and G455 of VR-IV (FIG. 9C), each with the TLAAPFK (SEQ ID NO:1) insert shown in bold.

FIGS. 10A-10B depict an in vitro transwell assay for AAV vectors crossing a blood brain barrier (BBB) cell layer (FIG. 10A), and results showing that AAV.hDyn (indicated by inverted triangles) crosses the BBB cell layer of the assay faster than AAV9 (squares), as well as faster and to a greater extent than AAV2 (circles) (FIG. 10B).

FIG. 11 depicts results of Next Generation Sequencing (NGS) analysis of brain gDNA from mice to which pools of engineered and native capsids have been intravenously administered, revealing relative abundances in tissues of the mice of the different capsids in the pool. Three different pools were injected into mice. Dotted lines indicate which vectors were pooled together. Parental AAV9 was included in each pool as control (Pool 1: BC01, Pool 2: BC31, Pool 3: BC01). Bar codes for each capsid of the pool are listed in Tables 6a-6c.

FIGS. 12A-12H depict an in vivo transduction profile of AAV.hDyn in female C57Bl/6 mice, showing copy number/microgram gDNA in naïve mice, or mice injected with either AAV9 or AAV.hDyn in brain (FIG. 12A), liver (FIG. 12B), heart (FIG. 12C), lung (FIG. 12D), kidney (FIG. 12E), skeletal muscle (FIG. 12F), sciatic nerve (FIG. 12G), and ovary (FIG. 12H), where AAV.hDyn shows increased brain bio-distribution compared to AAV9.

FIGS. 13A-13C depict distribution of GFP from AAV.hDyn throughout the brain, where images of immunohistochemical staining of brain sections from the striatum (FIG. 13A), hippocampus (FIG. 13B), and cortex (FIG. 13C) revealed a comprehensive transduction of the brain by the modified vector.

FIG. 14 depicts in vivo kidney to liver transduction efficiency ratio of genetically engineered AAV9 vectors containing insertions of homing peptides immediately after amino acid 454. Details on peptides used in this study are outlined in Table 6.

FIG. 15 depicts the relative abundance (RA) of the viral genomes (normalized to input) in the frontal cortex of the cynomolgus monkey model.

FIGS. 16 and 17 depict the Relative Abundance of the viral genomes (normalized to input) in the hippocampus and the cerebellum of the cynomolgus monkey model, respectively. The RA of AAV.rh34 is shown by the shaded column on the left side of the graphs and the RA of AAV9 reference is showed by the shaded column in the middle of the graphs. FIGS. 16 and 17 show that AAV.rh34 is a top performing capsid in the intravenous administration pool.

FIG. 18 depicts a Venn diagram of the: top 45 performers in FC (highest RA to AAV9), and bottom 45 performers in the cervical, thoracic, and lumbar DRGs (lowest RA to AAV9) and the AAVrh34 capsid is shown as the only capsid that was present in each group of 45 amongst the pool of capsids.

FIG. 19 depicts the RA of the viral genomes (normalized to input and AAV9 control) in the frontal cortex of the cynomolgus monkey model.

FIG. 20 depicts the RA of the viral genomes (normalized to input) in the hippocampus of the cynomolgus monkey model.

FIG. 21 depicts the RA of the viral genomes (normalized to input) in the midbrain of the cynomolgus monkey model.

FIG. 22 depicts the RA of the viral genomes (normalized to input) in the cerebellum of the cynomolgus monkey model.

FIG. 23 depicts the RA of the viral genomes (normalized to input) in the cervical DRGs of the cynomolgus monkey model.

FIG. 24 depicts the RA of the viral genomes (normalized to input) in the lumbar DRGs of the cynomolgus monkey model.

FIG. 25 depicts a Venn diagram of the top performing 15 capsids transducing the frontal cortex, hippocampus, midbrain and cerebellum following ICV administration. As indicated in the diagram, AAV6, AAV8.BBB, AAV.rh.46, and AAV1 were the only AAVs represented in each of the top performing groups.

FIG. 26 depicts a Venn diagram of the top performing 45 capsids transducing the hippocampus and the 45 capsids with the lowest transduction values for DRG, to identify hippocampus-targeting DRG friendly capsids. As indicated in the diagram, AAV.hu.60, AAV.rh.21, AAV.PHP.hB, AAV.rh.15, AAV.rh.24, AAV9.W503R, hu.5, AAV9.Q474A, and AAV.hu.10 were the only AAVs represented in each of the groups.

FIG. 27 depicts a Venn diagram of the top performing 40 capsids transducing the heart, biceps, and gastrocnemius and the 40 capsids with the lowest transduction values for the liver, to identify muscle-targeting capsids that have reduced targeting to the liver. As indicated in the diagram, AAV.PHPeB.VP2Herp was the only AAVs represented in each of the groups.

FIG. 28 depicts a Venn diagram of the top performing 15 capsids transducing the heart, biceps, and gastrocnemius muscle.

FIGS. 29A and B depict the RA of the viral genomes (normalized to input) in the gastrocnemius and the liver of the cynomolgus monkey model, respectively.

FIGS. 30A and 30B depict the number of genome copies of DNA (A) or RNA (B) of select “liver-detargeting” (LD) vectors as detected in the liver of NHPs following IV administration of the capsid library.

FIG. 31 depicts the biodistribution of select “liver-detargeting” (LD) vectors compared to their parental AAV9 capsid in various tissues, in NHPs following IV administration of the capsid library.

FIG. 32 depicts the biodistribution of select LD vectors compared to their parental AAV8 capsid in various tissues, in NHPs following IV administration of the capsid library.

FIGS. 33A and 33B. FIG. 33A depicts the change in relative abundance for point mutations affecting AAV9 transduction (or liver transduction) as compared to AAV9. The four mutants depicted in this study demonstrate retention in the blood when compared to wild-type AAV9. FIG. 33B shows the change in relative abundance for the AAV8 and AAV9 mutants combining the NNN/AAA mutation with the transport motif BBB (A269S, in AAV8; S263G/S269T/A273T, in AAV9), as compared to parental capsid (AAV8 or AAV9, respectively).

FIG. 34A and FIG. 34B. FIG. 34A shows the increase in blood retention 3-24 hr relative to AAV9. FIG. 34B shows the increase in blood retention 3-24 hr relative to parental AAV.

FIG. 35 depicts the biodistribution of select AAV vectors in muscle tissues, including cardiac muscle, as well as cerebrum, liver and pancreas in wild-type B6 mice following IV administration of the capsid library.

FIGS. 36A-36H depicts biodistribution of various capsids in wild-type B6 mice compared to that of mdx mouse tissue.

### 5. DETAILED DESCRIPTION

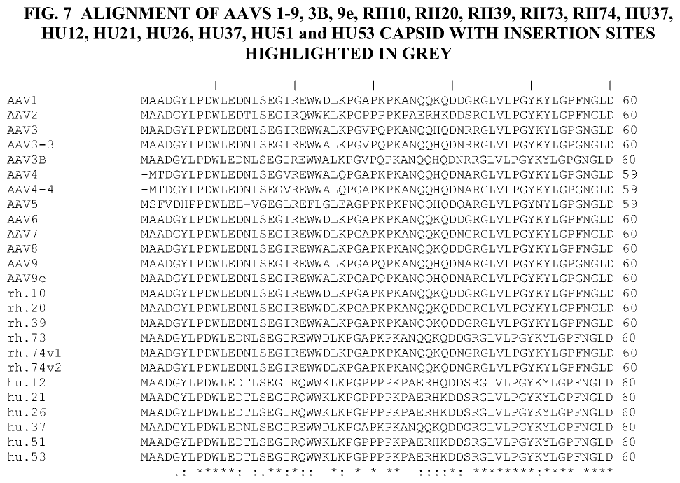

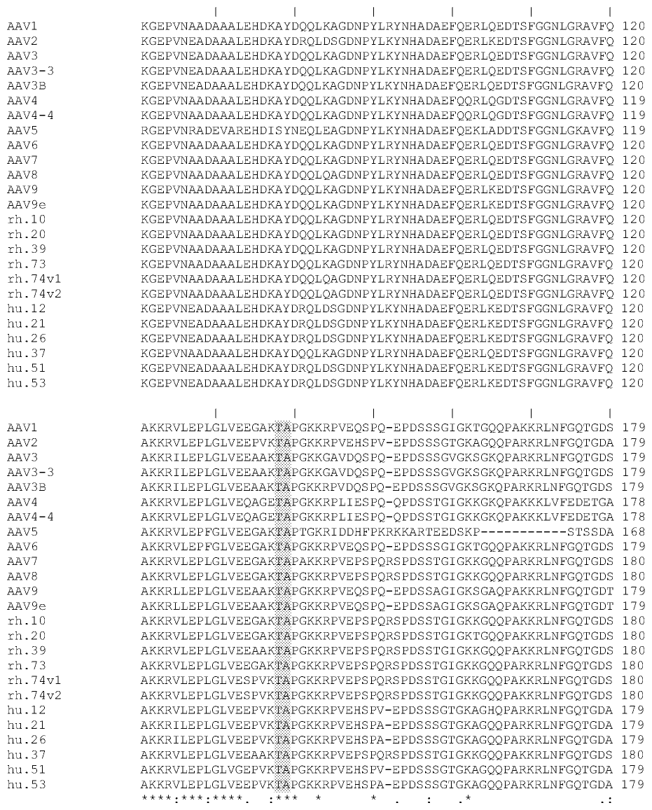

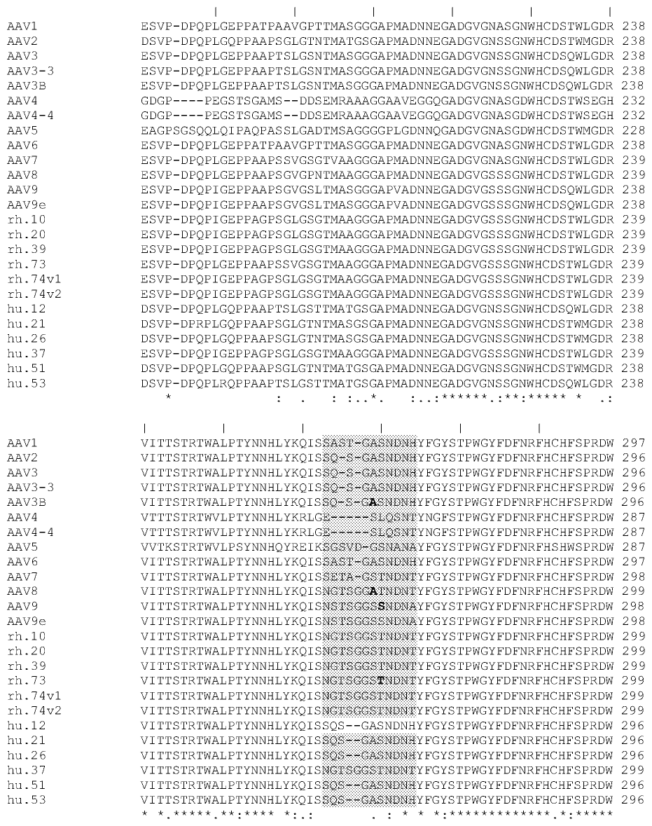

Provided are recombinant adeno-associated viruses (rAAVs) having capsid proteins engineered relative to a reference capsid protein, such that the rAAV has enhance desired properties, such as increased tissue targeting, including transduction, genome integration and transgene expression, particularly, preferentially, relative to the reference capsid protein (e.g., the unengineered or wild type capsid), to CNS or to heart and/or skeletal muscle tissue. In embodiments, the engineered capsid has reduced tropism (i.e., tissue targeting, transduction and integration of the rAAV genome) relative to the reference capsid for liver, dorsal root ganglion and/or peripheral nervous tissue to reduce toxicity of the AAV gene therapy. The modifications include amino acid substitutions (including 1, 2, 3, 4, 5, 6, 7 or 8 amino acid substitutions) and/or peptide insertions (4 to 20, or 7 contiguous amino acids, and in embodiments no more than 12 contiguous amino acids from a heterologous protein) as described herein. The AAV capsid protein to be engineered is, in certain embodiments, an AAV9 capsid protein or an AAV8 capsid protein. In other embodiments, the AAV capsid to be engineered is an AAV rh.34, AAV4, AAV5, AAV hu.26, AAV rh.31, AAV hu.13, AAV hu.56, AAV hu.53, AAV7, AAV rh64R1, AAV rh46 or AAV rh73 capsid protein. (See FIG. 7 and Table 17 for sequences)

Accordingly provided are engineered capsids having one or more amino acid substitutions that promote transduction and/or tissue tropism, particularly for enhanced, relative to an unengineered capsid, targeting for heart and/or skeletal muscle and, in embodiments, reduced, relative to an unengineered capsid, targeting for liver, dorsal root ganglion, and/or peripheral nervous tissue. In embodiments, the amino acid substitutions are S263F/S269T/A273T of AAV9, and corresponding substitutions in other AAV type capsids (for example according to the alignment in FIG. 7), or W530R, Q474A, N272A, or G266A of AAV9, and corresponding substitutions in other AAV type capsids or A269S of AAV8 and corresponding substitutions in other AAV capsids (for example, according to the alignment in FIG. 7). Also provided are capsids, particularly AAV9 capsids having a peptide TLAAPFK (SEQ ID NO:1) inserted between Q588 and A589 (herein PHP.hDYN) or alternatively between S268 and 5269 or between 5454 and G455) or inserted in another AAV capsid at a corresponding position (see, e.g., FIG. 7). Or, alternatively, the capsid is an AAV9 PHP.eB capsid (which has the modifications A587D and Q588G and insertion of the peptide TLAVPFK (SEQ ID NO:20) between G588 and A589) and the peptide TILSRSTQTG (SEQ ID NO:15) between position 138 and 139, or the corresponding. Additional capsids have a Kidney1 peptide LPVAS inserted into the capsid, for example between S454 and G455 of AAV9, or alternatively between S268 and S269 or between Q588 and A589, or the corresponding position of a different capsid. In some embodiments, the capsids can comprise R697W substitution of AAV rh64R1. The capsids having these amino acid substitutions and insertions may further have substitutions of the NNN (asparagines) at 496 to 498 with AAA (alanines) of the AAV9 capsid or at positions 498 to 500 of the AAV8 capsid, or corresponding substitutions in other AAV type capsids. Engineered capsids include AAV8.BB.LD (A269S,498-NNN/AAA-500 substitutions in the amino acid sequence of AAV8, SEQ ID NO 66), AAV9.BB.LD (S263G/S269T/A273T, 496-NNN/AAA-498 substitutions in the amino acid sequence of AAV9, SEQ ID NO 67), AAV9.496-NNN/AAA-498 (SEQ ID NO: 31), AAV9.496-NNN/AAA-498.W503R (SEQ ID NO: 32), AAV9.W503R (SEQ ID NO: 33), or AAV9.Q474A (SEQ ID NO: 34). In other examples, the capsid can be AAV9.N272A.496-NNN-498 (SEQ ID NO:91) or AAV9.G266A.496-NNN-498 (SEQ ID NO: 92). In other embodiments, the capsid is not an engineered capsid, but is an AAVrh.10 capsid (SEQ ID NO: 69), an AAVrh.46 capsid (SEQ ID NO:93), an AAVrh.64.R1 capsid (SEQ ID NO: 90) or an AAVrh.73 capsid (SEQ ID NO: 88). In certain embodiments, transduction is measured by detection of transgene, such as GFP fluorescence. The capsids having these amino acid substitutions and insertions may further have substitutions of the NNN (asparagines) at 496 to 498 with AAA (alanines) of the AAV9 capsid, or corresponding substitutions in other AAV type capsids. This engineered capsid may exhibit preferential targeting for heart and/or skeletal muscle, and reduced targeting (compared to an AAV bearing the unengineered capsid) for liver and/or dorsal root ganglion cells and/or peripheral nervous system tissue, and may particularly useful for delivery of a transgene encoding a therapeutic protein for treatment of a muscle disease.

In another embodiment, provided is a recombinant capsid protein, including an engineered AAV9 capsid protein, and an rAAV comprising the capsid protein, in which the peptide TLAVPFK (SEQ ID NO:20) is inserted between G588 and A589 of AAV9, and, in particular, the capsid protein also has amino acid substitutions A587D/Q588G (PHP.eB) and further has the peptide TILSRSTQTG (SEQ ID NO:15) inserted after position 138 of AAV9 (collectively, AAVPHPeB.VP2Herp; see Table 17), or in the corresponding positions of another AAV. Additional capsids have a Kidney1 peptide LPVAS (SEQ ID NO:6) (or alternatively CLPVASC (SEQ ID NO:5)) inserted into the capsid, for example between 5454 and G455 of AAV9 (see Table 17), or alternatively between 5268 and 5269 or between Q588 and A589, or the corresponding position of a different capsid. Such an engineered capsid may exhibit preferential targeting for heart and skeletal muscle, and reduced targeting (as compared to an AAV having the unengineered capsid) for liver and/or dorsal root ganglion cells and may particularly useful for delivery of a transgene encoding a therapeutic protein for treatment of a muscle disease (such as, but not limited to a muscular dystrophy).

In embodiments the engineered rAAV exhibits at least 1.1-fold, 1.5-fold, 2-fold, 3-fold, 4-fold, 5-fold, 6-fold, 7-fold, 8-fold, 9-fold, or 10-fold greater transduction in cardiac muscle and/or skeletal muscle cells compared to a reference AAV capsid, including an AAV9 capsid or an AAV8 capsid, or the parental capsid. In particular embodiments, the muscle is gastrocnemius muscle, bicep, tricep and/or heart muscle. In further embodiments, the engineered rAAV exhibits of 50%, 60%, 70%, 80%, 90%, 95% or 99% less transduction in liver compared to the reference AAV capsid compared to a reference AAV capsid, including an AAV9 capsid or an AAV8 capsid, or the parental capsid. In further embodiments, the rAAV exhibits of 50%, 60%, 70%, 80%, 90%, 95% or 99% less transduction in dorsal root ganglion cells (including in cervical, thoracic or lumbar DRG cells) compared to the reference AAV capsid. The enhanced and/or reduce transduction may be with any mode of administration, by intravenous administration, intramuscular administration, or any type of systemic administration, intrathecal administration or ICV administration.

Also provided are engineered capsids having one or more amino acid substitutions that promote transduction and/or tissue tropism, particularly for enhanced, relative to an unengineered capsid, targeting for CNS and, in embodiments, reduced, relative to an unengineered capsid, targeting for liver, dorsal root ganglion, and/or peripheral nervous tissue. In embodiments, the amino acid substitutions are A269S of AAV8 (or at a corresponding position in a different AAV serotype capsid), S263G/S269T/A273T of AAV9 (or at a corresponding position in a different AAV serotype capsid), N272A or N266A of AAV9 (or at a corresponding position in a different AAV serotype capsid), Q474A of AAV9 (or at a corresponding position in a different AAV serotype capsid), or W503R of AAV9 (or at a corresponding position in a different AAV serotype capsid), or R697W of rh64R1 (or at a corresponding position in a different AAV serotype capsid). The capsids having these amino acid substitutions and insertions may further have or alternatively have substitutions of the NNN (asparagines) at 496 to 498 with AAA (alanines) of the AAV9 capsid (SEQ ID NO: 67) or have substitutions of the NNN (asparagines) at 498 to 4500 with AAA (alanines) of the AAV8 capsid (SEQ ID NO: 66), or corresponding substitutions in other AAV type capsids. This engineered capsid may exhibit preferential targeting for CNS, and reduced targeting (compared to an AAV bearing the unengineered capsid) for liver and/or dorsal root ganglion cells and/or peripheral nervous system tissue, and may particularly useful for delivery of a transgene encoding a therapeutic protein for treatment of a CNS disease.

Also provided are recombinant capsid proteins, and rAAVs comprising them, that have inserted peptides that target and/or promote rAAV cellular uptake, transduction and/or genome integration in CNS tissue and, in embodiments, reduced, relative to an unengineered capsid, targeting for liver, dorsal root ganglion, and/or peripheral nervous tissue, for example, the peptide TILSRSTQTG (SEQ ID NO:15); TLAVPFK (SEQ ID NO:20); or TLAAPFK (SEQ ID NO:1). In particular embodiments the peptide TLAAPFK (SEQ ID NO:1) is inserted between Q588 and A589 of AAV9 (AAV9.hDyn; see Table 17), or the corresponding position of another AAV (see FIG. 7). Alternatively, the capsid is rh.34, rh.10, rh.46, rh.73, or rh64.R1 (FIG. 7 or Table 17 for sequence), or an engineered form of rh.34, rh.10, rh.46, rh.73, or rh64.R1. These engineered capsids may exhibit preferential targeting for CNS, and reduced targeting (compared to an AAV bearing the unengineered capsid) for liver and/or dorsal root ganglion cells and/or peripheral nervous system tissue, and may particularly useful for delivery of a transgene encoding a therapeutic protein for treatment of a CNS disease.

In embodiments the engineered rAAV exhibits at least 1.1-fold, 1.5-fold, 2-fold, 3-fold, 4-fold, 5-fold, 6-fold, 7-fold, 8-fold, 9-fold, or 10-fold greater transduction in CNS tissue compared to a reference AAV capsid, such as the parental capsid or AAV8 or AAV9. The CNS tissue may be one or more of the frontal cortex, hippocampus, cerebellum, midbrain and/or hindbrain. In further embodiments, the engineered rAAV exhibits of 50%, 60%, 70%, 80%, 90%, 95% or 99% less transduction in liver compared to the reference AAV capsid such as the parental capsid or AAV8 or AAV9. In further embodiments, the rAAV exhibits of 50%, 60%, 70%, 80%, 90%, 95% or 99% less transduction in dorsal root ganglion cells (including in cervical, thoracic or lumbar DRG cells) compared to the reference AAV capsid such as the parental capsid or AAV8 or AAV9. The enhanced and/or reduce transduction may be with any mode of administration, by intravenous administration, intramuscular administration, or any type of systemic administration, intrathecal administration or ICV administration.

Recombinant vectors comprising the capsid proteins also are provided, along with pharmaceutical compositions thereof, nucleic acids encoding the capsid proteins, and methods of making and using the capsid proteins and rAAV vectors having the engineered capsids for targeted delivery, improved transduction and/or treatment of disorders associated with the target tissue.

As used throughout, AAV “serotype” refers to an AAV having an immunologically distinct capsid, a naturally-occurring capsid, or an engineered capsid.

### 5.1. Definitions

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “subject”, “host”, and “patient” are used interchangeably. As used herein, a subject is a mammal such as a non-primate (e.g., cows, pigs, horses, cats, dogs, rats etc.) or a primate (e.g., monkey and human), or, in certain embodiments, a human.

As used herein, the terms “therapeutic agent” refers to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

As used herein, the term “prophylactic agent” refers to any agent which can be used in the prevention, delay, or slowing down of the progression of a disease or disorder, where the disease or disorder is associated with a function to be provided by a transgene. As used herein, a “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

The “central nervous system” (“CNS”) as used herein refers to neural tissue reaches by a circulating agent after crossing a blood-brain barrier, and includes, for example, the brain, optic nerves, cranial nerves, and spinal cord. The CNS also includes the cerebrospinal fluid, which fills the central canal of the spinal cord as well as the ventricles of the brain.

### 5.2. Recombinant AAV Capsids and Vectors

Provided are recombinant adeno-associated viruses (rAAVs) having capsid proteins engineered relative to a reference capsid protein, such that the rAAV has enhance desired properties, such as increased tissue targeting, including transduction, genome integration and transgene expression, particularly, preferentially, relative to the reference capsid protein (e.g., the unengineered or wild type capsid), to CNS or to heart and/or skeletal muscle tissue. In embodiments, the engineered capsid has reduced tropism (i.e., tissue targeting, transduction and integration of the rAAV genome) relative to the reference capsid for liver, dorsal root ganglion and/or peripheral nervous tissue to reduce toxicity of the AAV gene therapy. The modifications include amino acid substitutions (including 1, 2, 3, 4, 5, 6, 7 or 8 amino acid substitutions) and/or peptide insertions (4 to 20, or 7 contiguous amino acids, and in embodiments no more than 12 contiguous amino acids from a heterologous protein) as described herein.

5.2.1 Engineered Capsids with Amino Acid Substitutions

In some embodiments, AAV capsids were modified by introducing selected single to multiple amino acid substitutions which increase effective gene delivery to the CNS or to cardiac or skeletal muscle, detarget the liver and/or dorsal root ganglion to reduce toxicity, and/or reduce immune responses of neutralizing antibodies.

In particular embodiments the capsids have one or more amino acid substitutions including a W503R substitution, a Q474 substitutional a N272A or N266A substitution in AAV9 or the corresponding substitution in another AAV serotype or an A269S substitution in AAV8 or the corresponding substitution in another AAV serotype. rAAV having a capsid with the Q474A substitution may be particularly useful for delivery to skeletal and/or cardiac muscle or CNS tissue and rAAV having a capsid with the W503R substitution may be particularly useful for delivery to CNS tissue, particularly with reduced, compared to reference capsid containing rAAVs, transduction in the liver and/or DRGs. Other substitutions include S263G/S269R/A273T substitutions in AAV9 or A587D/Q588G in AAV9 or corresponding substitutions in other AAV serotypes. In some embodiments, the rAAV capsid can have a R697W substitution. The capsids having these amino acid substitutions and insertions may further have substitutions of the NNN (asparagines) at 496 to 498 with AAA (alanines) of the AAV9 capsid, or of the NNN (asparagines) at 498 to 500 with AAA (alanines) of the AAV8 capsid corresponding substitutions in other AAV type capsids. Other AAV serotypes that may be used for the amino acid substitutions and that may be the reference capsid include AAV8, AAV rh.34, AAV4, AAV5, AAV hu.26, AAV rh.31, AAV hu.13, AAV hu.26, AAV hu.56, AAV hu.53, AAV7, rh64R1, rh46 or rh73. In particular embodiments for CNS delivery, the capsid is rh34, either unmodified or serving as the parental capsid to be modified as detailed herein.

Effective gene delivery to the CNS by intravenously administered rAAV vectors requires crossing the blood brain barrier. Key clusters of residues on the AAVrh.10 capsid that enabled transport across the brain vasculature and widespread neuronal transduction in mice have recently been reported. Specifically, AAVrh.10-derived amino acids N262, G263, T264, S265, G267, 5268, T269, and T273 were identified as key residues that promote crossing the BBB (Albright et al, 2018, Mapping the Structural Determinants Required for AAVrh.10 Transport across the Blood-Brain Barrier). Amino acid substitutions in capsids, such as AAV8 and AAV9 capsids that promote rAAV crossing of the blood brain barrier, transduction, detargeting of the liver and/or reduction in immune responses have been identified.

In some embodiments, provided are capsids having one or more amino acid substitutions that promote transduction and/or tissue tropism of the rAAV having the modified capsid. In particular embodiments, provided are capsids having a single mutation at amino acid 269 of the AAV8 capsid replacing alanine with serine (A269S) (see, Tables 5a-5c, herein referred to as AAV8.BBB) and amino acid substitutions at corresponding positions in other AAV types. In some embodiments, provided are capsids having multiple substitutions at amino acids 263, 269, and 273 of the AAV9 capsid resulting in the following substitutions: S263G, S269T, and A273T (herein referred to as AAV9.BBB) or substitutions corresponding to these positions in other AAV types.

Exposure to the AAV capsid can generate an immune response of neutralizing antibodies. One approach to overcome this response is to map the AAV-specific neutralizing epitopes and rationally design an AAV capsid able to evade neutralization. A monoclonal antibody, specific for intact AAV9 capsids, with high neutralizing titer has recently been described (Giles et al, 2018, Mapping an Adeno-associated Virus 9-Specific Neutralizing Epitope To Develop Next-Generation Gene Delivery Vectors). The epitope was mapped to the 3-fold axis of symmetry on the capsid, specifically to residues 496-NNN-498 and 588-QAQAQT-592 of AAV9 (SEQ ID NO:8). Capsid mutagenesis demonstrated that single amino acid substitution within this epitope markedly reduced binding and neutralization. In addition, in vivo studies showed that mutations in the epitope conferred a “liver-detargeting” phenotype to the mutant vectors, suggesting that the same residues are also responsible for AAV9 tropism. Liver detargeting has also been associated with substitution of amino acid 503 replacing tryptophan with arginine. Presence of the W503R mutation in the AAV9 capsid was associated with low glycan binding avidity (Shen et al, 2012, Glycan Binding Avidity Determines the Systemic Fate of Adeno-Associated Virus Type 9).

In some embodiments, provided are capsids in which the AAV8.BBB and AAV9.BBB capsids were further modified by substituting asparagines at amino acid positions 498, 499, and 500 of AAV8 (herein referred to as AAV8.BBB.LD) or 496, 497, and 498 of AAV9 (herein referred to as AAV9.BBB.LD) with alanines. In some embodiments, the AAVrh10 capsid was modified by substituting three asparagines at amino acid positions 498, 499, and 500 to alanines (AAVrh10.LD) (Tables 5a-5c).

In some embodiments, provided are capsids having three asparagines at amino acid positions 496, 497, and 498 of the AAV9 capsid replaced with alanines and also tryptophan at amino acid 503 of the AAV9 capsid with arginine or capsids with substitutions corresponding to these positions in other AAV types. In some embodiments, provided are capsids having glutamine at amino acid position 474 of the AAV9 capsid substituted with alanine or capsids with substitutions corresponding to this position in other AAV types.

In some embodiments, the capsid is an AAV8.BB.LD capsid (A269S,498-NNN/AAA-500 substitutions in the amino acid sequence of AAV8, SEQ ID NO 66), an AAV9.BBB.LD capsid (S263G/S269T/A273T, 496-NNN/AAA-498 substitutions in the amino acid sequence of AAV9, SEQ ID NO 67), an AAV9.496-NNN/AAA-498 capsid (SEQ ID NO: 31), an AAV9.496-NNN/AAA-498.W503R capsid (SEQ ID NO: 32), an AAV9.W503R capsid (SEQ ID NO: 33), or an AAV9.Q474A capsid (SEQ ID NO: 34). In other examples, the capsid can be an AAV9.N272A.496-NNN-498 capsid (SEQ ID NO:91) or an AAV9.G266A.496-NNN-498 capsid (SEQ ID NO: 92).

In some embodiments, the rAAVs described herein increase tissue-specific (such as, but not limited to, CNS or skeletal and/or cardiac muscle) cell transduction in a subject (a human, non-human-primate, or mouse subject) or in cell culture, compared to the rAAV not comprising the amino acid substitution. In some embodiments, the increase in tissue specific cell transduction is at least 2, 10, 20, 30, 40, 50, 60, 70, 80, 90, or 100 fold more than that without the modification. For example, in some embodiments, there is a 50-80 fold increase in tissue specific cell transduction compared to transduction with the same AAV type without the modification. The increase in transduction may be assessed using methods described in the Examples herein and known in the art.

In some embodiments, the rAAVs described herein increase the incorporation of rAAV genomes into a cell or tissue type in a subject (a human, non-human primate or mouse subject) or in cell culture to the rAAV not comprising the peptide insertion. In some embodiments, the increase in genome integration is at least 2, 10, 20, 30, 40, 50, 60, 70, 80, 90, or 100 fold more than an AAV having a capsid without the modification (i.e., the parental capsid). For example, in some embodiments, there is a 50-80 fold increase in genome integration compared to genome integration with the same AAV type without the modification.

5.2.2 rAAV Vectors with Peptide Insertions

Provided are rAAVs having capsid proteins with one or more (generally one or two) peptide insertions wherein the peptide insertion increase effective gene delivery to the CNS or to cardiac or skeletal muscle and to detarget the liver and/or dorsal root ganglion to reduce toxicity relative to the parental capsid protein. In particular embodiments, the peptides include TLAVPFK, TLAAPFK, or TILSRSTQTG (or an at least 4, 5, 6, 7 amino acid portion thereof). The peptides may be inserted into the AAV9 capsid, for example after the positions 138; 262-273; 452-461; 585-593 of AAV9 cap, particularly after position 138, 454 or 588 of AAV9 or a corresponding position in another AAV as detailed herein. In particular embodiments, the capsid has the peptide TLAVPFK (SEQ ID NO:20) is inserted between G588 and A589 of AAV9, and, in particular, the capsid protein also has amino acid substitutions A587D/Q588G (PHP.eB) and further has the peptide TILSRSTQTG (SEQ ID NO:15) inserted after position 138 of AAV9 (collectively, AAVPHPeB.VP2Herp; see Table 17), or in the corresponding positions of another AAV. Additional capsids have a Kidney1 peptide LPVAS inserted into the capsid, for example between 454 and 455 of AAV9 (see Table 17), or alternatively or alternatively between 5268 and 5269 or between Q588 and A589 of AAV9 or the corresponding position of another AAV serotype. Such an engineered capsid may exhibit preferential targeting for heart and skeletal muscle, and reduced targeting (as compared to an AAV having the unengineered capsid) for liver and/or dorsal root ganglion cells and may particularly useful for delivery of a transgene encoding a therapeutic protein for treatment of a muscle disease (such as, but not limited to a muscular dystrophy).

In some embodiments, the peptide insertion comprises at least 4, 5, 6, 7, 8, 9, or all 10 consecutive amino acids of sequence TILSRSTQTG (SEQ ID NO:15), preferably which contains the TQT or STQT (SEQ ID NO:9) motif. In some embodiments, the peptide insertion consists of at least 4, 5, 6, 7, 8, 9, or all 10 consecutive amino acids of sequence TILSRSTQTG (SEQ ID NO:15), preferably which contains the TQT or STQT (SEQ ID NO:9) motif.

In certain embodiments, the peptide insertion may be a sequence of consecutive amino acids from a domain that targets kidney tissue, or a conformation analog designed to mimic the three-dimensional structure of said domain. In some embodiments, the kidney-homing domain comprises the sequence CLPVASC (SEQ ID NO:5) (see, e.g., U.S. Pat. No. 5,622,699). In some embodiments, the peptide insertion from said kidney-homing domain comprises at least 4, 5, 6, or all 7 amino acids from sequence CLPVASC (SEQ ID NO:5). In some embodiments, the peptide insertion comprises or consists of the sequence CLPVASC (SEQ ID NO:5).

It has been found that both of the cysteine residues in certain homing peptides can be deleted without significantly affecting the organ homing activity of the peptide. For example, a peptide having the sequence LPVAS (SEQ ID NO:6) also can be a kidney-homing peptide. Methods for determining the necessity of a cysteine residue or of amino acid residues N-terminal or C-terminal to a cysteine residue for organ homing activity of a peptide are routine and well known in the art. Thus, in some embodiments, the peptide insertion comprises at least 4 or all 5 amino acids from sequence LPVAS (SEQ ID NO:6). In some embodiments, the peptide insertion comprises or consists of the sequence LPVAS (SEQ ID NO:6).

In particular embodiments, provided are rAAVs having a capsid that has the peptide TLAAPFK (SEQ ID NO:1) is inserted between Q588 and A589 of AAV9 (AAV9.hDyn; see Table 17), or the corresponding position of another AAV (see, e.g., FIG. 7). Such an engineered capsid may exhibit preferential targeting for CNS tissue, and reduced targeting (as compared to an AAV having the unengineered capsid) for liver and/or dorsal root ganglion cells and may particularly useful for delivery of a transgene encoding a therapeutic protein for treatment of a CNS disease.

Provided are capsids with peptide insertions at positions amenable to peptide insertions within and near the AAV9 capsid VR-IV loop (see FIG. 2) and corresponding regions on the VR-IV loop of capsids of other AAV types. Though previous studies analyzed potential positions in various AAVs, none identified the AAV9 VR-IV as amenable for this purpose (consider, e.g., Wu et al, 2000, “Mutational Analysis of the Adeno-Associated Virus Type 2 (AAV2) Capsid Gene and Construction of AAV2 Vectors with Altered Tropism,” J of Virology 74(18):8635-8647; Lochrie et al, 2006, “Adeno-associated virus (AAV) capsid genes isolated from rat and mouse liver genomic DNA define two new AAV species distantly related to AAV-5,” Virology 353:68-82; Shi and Bartlett, 2003, “RGD Inclusion in VP3 Provides Adeno-Associated Virus Type 2 (AAV2)-Based Vectors with a Heparan Sulfate-Independent Cell Entry Mechanism,” Molecular Therapy 7(4):515525-; Nicklin et al., 2001, “Efficient and Selective AAV2-Mediated Gene Transfer Directed to Human Vascular Endothelial Cells” Molecular Therapy 4(2):174-181; Grifman et al., 2001, “Incorporation of Tumor-Targeting Peptides into Recombinant Adeno-associated Virus Capsids,” Molecular Therapy 3(6):964-975; Girod et al. 1999, “Genetic capsid modifications allow efficient re-targeting of adeno-associated virus type 2,” Nature Medicine 3(9):1052-1056; Douar et al., 2003, “Deleterious effect of peptide insertions in a permissive site of the AAV2 capsid, “Virology 309:203-208; and Ponnazhagan, et al. 2001, J. of Virology 75(19):9493-9501).

Accordingly, provided are rAAV vectors carrying peptide insertions at these points, in particular, within surface-exposed variable regions in the capsid coat, particularly within or near the variable region IV of the capsid protein. In some embodiments, the rAAV capsid protein comprises a peptide insertion immediately after (i.e., connected by a peptide bond C-terminal to) an amino acid residue corresponding to one of amino acids 451 to 461 of AAV9 capsid protein (amino acid sequence SEQ ID NO:67 and see FIG. 7 for alignment of capsid protein amino acid sequence of other AAV serotypes with amino acid sequence of the AAV9 capsid and Table 17 for other capsid sequences), where said peptide insertion is surface exposed when the capsid protein is packaged as an AAV particle. The peptide insertion should not delete any residues of the AAV capsid protein. Generally, the peptide insertion occurs in a variable (poorly conserved) region of the capsid protein, compared with other serotypes, and in a surface exposed loop.

A peptide insertion described as inserted “at” a given site refers to insertion immediately after, that is having a peptide bond to the carboxy group of, the residue normally found at that site in the wild type virus. For example, insertion at Q588 in AAV9 means that the peptide insertion appears between Q588 and the consecutive amino acid (A589) in the AAV9 wildtype capsid protein sequence (SEQ ID NO:67). In embodiments, there is no deletion of amino acid residues at or near (within 5, 10, 15 residues or within the structural loop that is the site of the insertion) the point of insertion.

In particular embodiments, the capsid protein is an AAV9 capsid protein and the insertion occurs immediately after at least one of the amino acid residues 451 to 461. In particular embodiments, the peptide insertion occurs immediately after amino acid 1451, N452, G453, 5454, G455, Q456, N457, Q458, Q459, T460, or L461 of the AAV9 capsid (amino acid sequence SEQ ID NO:67). In certain embodiments, the peptide is inserted between residues S454 and G455 of AAV9 capsid protein or between the residues corresponding to S454 and G455 of an AAV capsid protein other than an AAV9 capsid protein (amino acid sequence SEQ ID NO:67).

In other embodiments, provided are engineered capsid proteins comprising targeting peptides heterologous to the capsid protein that are inserted into the AAV capsid protein such that, when incorporated into the AAV vector the heterologous peptide is surface exposed.

In other embodiments, the capsid protein is from at least one AAV type selected from AAV serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype hu.37 (AAVhu.37), serotype rh74 (AAVrh74, versions 1 and 2), serotype rh34 (AAVrh34), serotype hu26 (AAVhu26), serotype rh31 (AAVrh31), serotype hu56 (AAVhu56), serotype hu53 (AAVhu53), serotype rh64R1 (AAVrh64R1), serotype rh46 (AAVrh46), and serotype rh73 (AAVrh73) (see FIG. 7 or Table 17), and the insertion occurs immediately after an amino acid residue corresponding to at least one of the amino acid residues 451 to 461. The alignments of these different AAV serotypes, as shown in FIG. 7, indicates “corresponding” amino acid residues in the different capsid amino acid sequences such that a “corresponding” amino acid residue is lined up at the same position in the alignment as the residue in the reference sequence. In some particular embodiments, the peptide insertion occurs immediately after one of the amino acid residues within: 450-459 of AAV1 capsid (SEQ ID NO:59); 449-458 of AAV2 capsid (SEQ ID NO:60); 449-459 of AAV3 capsid (SEQ ID NO:61); 443-453 of AAV4 capsid (SEQ ID NO:62); 442-445 of AAV5 capsid (SEQ ID NO:63); 450-459 of AAV6 capsid (SEQ ID NO:64); 451-461 of AAV7 capsid (SEQ ID NO:65); 451-461 of AAV8 capsid (SEQ ID NO:66); 451-461 of AAV9 capsid (SEQ ID NO:67); 452-461 of AAV9e capsid (SEQ ID NO:68); 452-461 of AAVrh10 capsid (SEQ ID NO:69); 452-461 of AAVrh20 capsid (SEQ ID NO:70); 452-461 of AAVhu.37 (SEQ ID NO:71); 452-461 of AAVrh74 (SEQ ID NO:72 or SEQ ID NO:80); or 452-461 of AAVrh39 (SEQ ID NO:73), in the sequences depicted in FIG. 7. In certain embodiments, the rAAV capsid protein comprises a peptide insertion immediately after (i.e., C-terminal to) amino acid 588 of AAV9 capsid protein (having the amino acid sequence of SEQ ID NO:67 and see FIG. 7), where said peptide insertion is surface exposed when the capsid protein is packaged as an AAV particle. In other embodiments, the rAAV capsid protein has a peptide insertion that is not immediately after amino acid 588 of AAV9 or corresponding to amino acid 588 of AAV9.

In specific embodiments, the peptide is inserted after 138; 262-272; 450-459; or 585-593 of AAV1 capsid (SEQ ID NO:59); 138; 262-272; 449-458; or 584-592 of AAV2 capsid (SEQ ID NO:60); 138; 262-272; 449-459; or 585-593 of AAV3 capsid (SEQ ID NO:61); 137; 256-262; 443-453; or 583-591 of AAV4 capsid (SEQ ID NO:62); 137; 252-262; 442-445; or 574-582 of AAV5 capsid (SEQ ID NO:63); 138; 262-272; 450-459; 585-593 of AAV6 capsid (SEQ ID NO:64); 138; 263-273; 451-461; 586-594 of AAV7 capsid (SEQ ID NO:65); 138; 263-274; 452-461; 587-595 of AAV8 capsid (SEQ ID NO:66); 138; 262-273; 452-461; 585-593 of AAV9 capsid (SEQ ID NO:67); 138; 262-273; 452-461; 585-593 of AAV9e capsid (SEQ ID NO:68); 138; 263-274; 452-461; 587-595 of AAVrh10 capsid (SEQ ID NO:69); 138; 263-274; 452-461; 587-595 of AAVrh20 capsid (SEQ ID NO:70); 138; 263-274; 452-461; 587-595 of AAVrh74 capsid (SEQ ID NO:72 or SEQ ID NO:80), 138; 263-274; 452-461; 587-595 of AAVhu37 capsid (SEQ ID NO:71); or 138; 263-274; 452-461; 587-595 of AAVrh39 capsid (SEQ ID NO:73) (as numbered in FIG. 7).

Generally, the peptide insertion is sequence of contiguous amino acids from a heterologous protein or domain thereof. The peptide to be inserted typically is long enough to retain a particular biological function, characteristic, or feature of the protein or domain from which it is derived. The peptide to be inserted typically is short enough to allow the capsid protein to form a coat, similarly or substantially similarly to the native capsid protein without the insertion. In preferred embodiments, the peptide insertion is from about 4 to about 30 amino acid residues in length, about 4 to about 20, about 4 to about 15, about 5 to about 10, or about 7 amino acids in length. The peptide sequences for insertion are at least 4 amino acids in length and may be 5, 6, 7, 8, 9, 10, 11, 12, 13, 14, or 15 amino acids in length. In some embodiments, the peptide sequences are 16, 17, 18, 19, or 20 amino acids in length. In embodiments, the peptide is no more than 7 amino acids, 10 amino acids or 12 amino acids in length.

A “peptide insertion from a heterologous protein” in an AAV capsid protein refers to an amino acid sequence that has been introduced into the capsid protein and that is not native to any AAV serotype capsid. Non-limiting examples include a peptide of a human protein in an AAV capsid protein.

In some embodiments, the rAAVs described herein increase tissue-specific (such as, but not limited to, CNS or skeletal and/or cardiac muscle) cell transduction in a subject (a human, non-human-primate, or mouse subject) or in cell culture, compared to the rAAV not comprising the amino acid substitution. In some embodiments, the increase in tissue specific cell transduction is at least 2, 10, 20, 30, 40, 50, 60, 70, 80, 90, or 100 fold more than that without the peptide insertion. For example, in some embodiments, there is a 50-80 fold increase in tissue specific cell transduction compared to transduction with the same AAV type without the modification. The increase in transduction may be assessed using methods described in the Examples herein and known in the art.

In some embodiments, the rAAVs described herein increase the incorporation of rAAV genomes into a cell or tissue type, particularly CNS or heart and/or skeletal muscle in a subject (a human, non-human primate or mouse subject) or in cell culture to the rAAV not comprising the peptide insertion. In some embodiments, the increase in genome integration is at least 2, 10, 20, 30, 40, 50, 60, 70, 80, 90, or 100 fold more than an AAV having a capsid without the peptide insertion. For example, in some embodiments, there is a 50-80 fold increase in genome integration compared to genome integration with the same AAV type without a peptide insert.

In another aspect, provided are libraries of capsids, including heterologous peptide insertion libraries or libraries of capsids having one or more amino acid substitutions. A heterologous peptide insertion library refers to a collection of rAAV vectors that carry the same peptide insertion at different insertion sites in the virus capsid, e.g., at different positions within a given variable region of the capsid or different variant peptides or even one or more amino acid substitutions. Provided are methods of screening the rAAVs having capsids from the library for enhance of improved properties such as tissue tropism, including enhanced transduction in CNS or cardiac and/or skeletal muscle tissue and, including, reduced transduction in liver and/or DRG cells. Generally, the capsid proteins used comprise AAV genomes that contain modified rep and cap sequences to prevent the replication of the virus under conditions in which it could normally replicate (co-infection of a mammalian cell along with a helper virus such as adenovirus). The members of the peptide insertion libraries may then be assayed for functional display of the peptide on the rAAV surface, tissue targeting and/or gene transduction.

### 5.2.3 Additional AAV Capsid Insertion Sites

The follow summarizes insertion sites for the peptides described herein immediately after amino acid residues of AAV capsids as set forth below (see also, FIG. 7):
AAV1: 138; 262-272; 450-459; 595-593; and in a particular embodiment, between 453-454 (SEQ ID NO:59).
AAV2: 138; 262-272; 449-458; 584-592; and in particular embodiment, between 452-453 (SEQ ID NO:60).
AAV3: 138; 262-272; 449-459; 585-593; and in particular embodiment, between 452-453 (SEQ ID NO:61).
AAV4: 137; 256-262; 443-453; 583-591; and in particular embodiment, between 446-447 (SEQ ID NO:62).
AAV5: 137; 252-262; 442-445; 574-582; and in particular embodiment, between 445-446 (SEQ ID NO:63).
AAV6: 138; 262-272; 450-459; 585-593; and in particular embodiment, between 452-453 (SEQ ID NO:64).
AAV7: 138; 263-273; 451-461; 586-594; and in particular embodiment, between 453-454 (SEQ ID NO:65).
AAV8: 138; 263-274; 451-461; 587-595; and in particular embodiment, between 453-454 (SEQ ID NO:66).
AAV9: 138; 262-273; 452-461; 585-593; and in particular embodiment, between 454-455 (SEQ ID NO:67).
AAV9e: 138; 262-273; 452-461; 585-593; and in particular embodiment, between 454-455 (SEQ ID NO:68).
AAVrh10: 138; 263-274; 452-461; 587-595; and in particular embodiment, between 454-455 (SEQ ID NO:69).
AAVrh20: 138; 263-274; 452-461; 587-595; and in particular embodiment, between 454-455 (SEQ ID NO:70).
AAVrh39: 138; 263-274; 452-461; 587-595; and in particular embodiment, between 454-455 (SEQ ID NO:73).
AAVrh74: 138; 263-274; 452-461; 587-595; and in particular embodiment, between 454-455 (SEQ ID NO:72 or SEQ ID NO:80).
AAVhu.37: 138; 263-274; 452-461; 587-595; and in particular embodiment, between 454-455 (SEQ ID NO:71)

In particular embodiments, the peptide insertion occurs between amino acid residues 588-589 of the AAV9 capsid, or between corresponding residues of another AAV type capsid as determined by an amino acid sequence alignment (for example, as in FIG. 7). In particular embodiments, the peptide insertion occurs immediately after amino acid residue 1451 to L461, S268 and Q588 of the AAV9 capsid sequence, or immediately after corresponding residues of another AAV capsid sequence (FIG. 7).

In some embodiments, one or more peptide insertions can be used in a single system. In some embodiments, the capsid is chosen and/or further modified to reduce recognition of the AAV particles by the subject's immune system, such as avoiding pre-existing antibodies in the subject. In some embodiments. In some embodiments, the capsid is chosen and/or further modified to enhance desired tropism/targeting.

5.2.4 AAV Vectors

Also provided are AAV vectors comprising the engineered capsids. In some embodiments, the AAV vectors are non-replicating and do not include the nucleotide sequences encoding the rep or cap proteins (these are supplied by the packaging cells in the manufacture of the rAAV vectors). In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAVrh34, AAVhu26, AAVrh31, AAVhu56, AAVhu53, AAVrh64R1, AAVrh46, and AAVrh73, or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, AAVrh34, AAVhu26, AAVrh31, AAVhu56, AAVhu53, AAVrh64R1, AAVrh46, and AAVrh73, or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV. rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSCS, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, AAVrh34, AAVhu26, AAVrh31, AAVhu56, AAVhu53, AAVrh64R1, AAVrh46, and AAVrh73, or a derivative, modification, or pseudotype thereof. These engineered AAV vectors may comprise a genome comprising a transgene encoding a therapeutic protein.

In particular embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLKO3 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSCS, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) may be used. In certain embodiments, a self-complementary vector, e.g., scAAV, may be used (see, e.g., Wu, 2007, Human Gene Therapy, 18(2):171-82; McCarty et al, 2001, Gene Therapy, 8(16):1248-1254; U.S. Pat. Nos. 6,596,535; 7,125,717; and 7,456,683, each of which is incorporated herein by reference in its entirety).

5.3. Methods of Making rAAV Particles

Another aspect of the present invention involves making rAAV particles having the capsids disclosed herein. In some embodiments, an rAAV particle is made by providing a nucleotide comprising the nucleic acid sequence encoding any of the capsid proteins described herein; and using a packaging cell system to prepare corresponding rAAV particles with capsid coats made up of the capsid protein. In some embodiments, the nucleic acid sequence encodes a sequence having at least 60%, 70%, 80%, 85%, 90%, 91%, 92%, 93%, 94%, 95%, 96%, 97%, 98%, 99% or 99.9%, identity to the sequence of a capsid protein molecule described herein, and retains (or substantially retains) biological function of the capsid protein. In some embodiments, the nucleic acid encodes a sequence having at least 60%, 70%, 80%, 85%, 90%, 91%, 92%, 93%, 94%, 95%, 96%, 97%, 98%, 99% or 99.9%, identity to the sequence of the one of the capsid proteins described herein, for example, those with sequences in Table 17 or otherwise described herein (see also FIG. 7), while retaining (or substantially retaining) biological function of the capsid protein.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In other embodiments, the cap and rep genes are provided by a packaging cell and not present in the viral genome.

In some embodiments, the nucleic acid encoding the engineered capsid protein is cloned into an AAV Rep-Cap helper plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In some embodiments, the rAAVs provide transgene delivery vectors that can be used in therapeutic and prophylactic applications, as discussed in more detail below. In some embodiments, the rAAV vector also includes regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue. In specific embodiments, the AAV vector comprises a regulatory sequence, such as a promoter, operably linked to the transgene that allows for expression in target tissues. The promoter may be a constitutive promoter, for example, the CB7 promoter. Additional promoters include: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter, opsin promoter, the TBG (Thyroxine-binding Globulin) promoter, the APOA2 promoter, SERPINA1 (hAAT) promoter, or MIR122 promoter. In some embodiments, particularly where it may be desirable to turn off transgene expression, an inducible promoter is used, e.g., hypoxia-inducible or rapamycin-inducible promoter.

Provided in particular embodiments are AAV vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the a capsid protein described herein (see Table 17, e.g.), while retaining the biological function of the engineered capsid. In certain embodiments, the encoded engineered capsid has the sequence of an AAV8.BBB.LD capsid (SEQ ID NO: 27), an AAV9.BBB.LD capsid (SEQ ID NO: 29), an AAV9.496-NNN/AAA-498 capsid (SEQ ID NO: 31), AAV9.496-NNN/AAA-498 capsid (SEQ ID NO: 32), AAV9.W503R capsid (SEQ ID NO: 33), AAV9.Q474A capsid (SEQ ID NO: 34), AAV9.N272A.496-NNN/AAA-498 capsid (SEQ ID NO: 91) or AAV9.N266A.496-NNN/AAA-498 capsid (SEQ ID NO: 92). Also provided are engineered AAV vectors other than AAV9 vectors, such as engineered AAV1, AAV2, AAV3, AAV4, AAV5, AAV6, AAV7, AAV8, AAV9, AAV9e, AAVrh10, AAVrh20, AAVhu.37, AAVrh39, AAVrh74, AAVrh34, AAVhu26, AAVrh31, AAVhu56, AAVhu53, AAVrh.46, AAVrh.64.R1, AAV.rh.73 vectors, including with the amino acid substitutions and/or peptide insert as described herein and 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions relative to the wild type or unengineered sequence for that AAV type and that retains its biological function.

The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety

The rAAV vector for delivering the transgene to target tissues, cells, or organs, has a tropism for that particular target tissue, cell, or organ. Tissue-specific promoters may also be used. The construct further can include expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) control elements, which include a) a promoter and, optionally, enhancer elements to promote expression of the transgene in CNS and/or muscle cells, b) optionally an intron sequence, such as a chicken β-actin intron, and c) a polyadenylation sequence, such as an SV40 polyA or rabbit β-globin poly A signal; and (3) transgene providing (e.g., coding for) a nucleic acid or protein product of interest.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COST, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or other cell types may be used, for example, but not limited, to HuH-7, HEK293, fibrosarcoma HT-1080, HKB-11, and CAP cells. Once expressed, characteristics of the expressed product (i.e., transgene product) can be determined, including determination of the glycosylation and tyrosine sulfation patterns, using assays known in the art.

### 5.4. Therapeutic and Prophylactic Uses

Another aspect relates to therapies which involve administering a transgene via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

Generally, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

Tables 1A-1B below provides a list of transgenes that may be used in any of the rAAV vectors described herein, in particular, in the novel insertion sites described herein, to treat or prevent the disease with which the transgene is associated, also listed in Tables 1A-1B. As described herein, the AAV vector may be engineered as described herein to target the appropriate tissue for delivery of the transgene to effect the therapeutic or prophylactic use. The appropriate AAV serotype may be chosen to engineer to optimize the tissue tropism and transduction of the vector.

TABLE 1A

| | | Possible |
|---|---|---|
| | | AAV |
| | | serotype for |
| | | delivery of |
| Disease | Transgene | transgene |
| MPS I | alpha-L-iduronidase (IDUA) | AAV9 |
| MPS II (Hunter | iduronate-2-sulfatase (IDS) | AAV9 |
| Syndrome) | | |
| ceroid lipofuscinosis | (CLN1, CLN2, CLN10, CLN13), a soluble | AAV9 |
| (Batten disease) | lysosomal protein (CLN5), a protein in the | |
| | secretory pathway (CLN11), two cytoplasmic | |
| | proteins that also peripherally associate with | |
| | membranes (CLN4, CLN14), and many | |
| | transmembrane proteins with different subcellular | |
| | locations (CLN3, CLN6, CLN7, CLN8, CLN12) | |
| MPS IIIa (Sanfilippo | heparan sulfate sulfatase (also called N- | AAV9, Rh10 |
| type A Syndrome) | sulfoglucosamine sulfohydrolase (SGSH)) | |
| MPS IIIB (Sanfilippo | N-acetyl-alpha-D-glucosaminidase (NAGLU) | AAV9 |
| type B Syndrome) | | |
| MPS VI (Maroteaux- | arylsulfatase B | AAV8 |
| Lamy Syndrome) | | |
| Morquio syndrome | Beta galactosidase or galactosamine-6-sulfatase | AAV9 |
| (MPS IV) | | |
| Gaucher disease (type | Glucocerebrosidase, GBA1 | AAV9 |
| 1, II and III) | | |
| Parkinson's Disease | Glucocerebrosidase; GBA1 | AAV9 |
| Parkinson's Disease | dopamine decarboxylase | AAV2 |
| Pompe | acid maltase; GAA | AAV9 |
| Metachromatic | Aryl sulfatase A | Rh10 |
| leukodystrophy | | |
| MPS VII (Sly | beta-glucuronidase | |
| syndrome) | | |
| MPS VIII | glucosamine-6-sulfate sulfatase | |
| MPS IX | hyaluronidase | |
| Niemann-Pick disease | sphingomyelinase | |
| Niemann-Pick disease | a npc1 gene encoding a | |
| without | cholesterol metabolizing enzyme | |
| sphingomyelinase | | |
| deficiency | | |
| Tay-Sachs disease | Alpha subunit of beta-hexosaminidase | |
| Sandhoff disease | both alpha and beta subunit of beta- | |
| | hexosaminidase | |
| Fabry Disease | alpha-galactosidase | |
| Fucosidosis | Fucosidase (FUCA1 gene) | |
| Alpha-mannosidosis | alpha-mannosidase | |
| Beta-mannosidosis | Beta-mannosidase | |
| Wolman disease | cholesterol ester hydrolase | |
| Parkinson's disease | Neurturin | |
| Parkinson's disease | glial derived growth factor (GDGF) | |
| Parkinson's disease | tyrosine hydroxylase | |
| Parkinson's disease | glutamic acid decarboxylase. | |
| No disease listed | fibroblast growth factor-2 (FGF-2) | |
| No disease listed | brain derived growth factor (BDGF) | |
| No disease listed | neuraminidase deficiency with betagalactosidase | |
| (Galactosialidosis | deficiency | |
| (Goldberg syndrome)) | | |
| Spinal Muscular | SMN | AAV9 |
| Atrophy (SMA) | | |
| Friedreich's ataxia | Frataxin | AAV9 |
| | | PHP.B |
| Amyotrophic lateral | SOD1 | Rh10 |
| sclerosis (ALS) | | |
| Glycogen Storage | Glucose-6-phosphatase | AAV8 |
| Disease 1a | | |
| XLMTM | MTM1 | AAV8 or |
| | | AAV9 |
| Crigler Najjar | UGT1A1 | AAV8 |
| CPVT | CASQ2 | AAV9 |
| Rett syndrome | MECP2 | AAV9 |
| Achromatopsia | CNGB3, CNGA3, GNAT2, PDE6C | AAV8 |
| Choroidermia | CDM | AAV8 |
| Danon Disease | LAMP2 | AAV9 |

TABLE 1B

| | | Possible AAV |
|---|---|---|
| | | serotype for |
| | | delivery of |
| Disease | Transgene | transgene |
| Cystic Fibrosis | CFTR | AAV2 |
| Duchenne Muscular Dystrophy | Mini-Dystrophin or Micro- | AAV2, AAV8 |
| | dystrophin Gene | or AAV9 |
| Limb Girdle Muscular Dystrophy Type | human-alpha-sarcoglycan | AAV1 |
| 2C\|Gamma-sarcoglycanopathy | | |
| Advanced Heart Failure | SERCA2a | AAV6 |
| Rheumatoid Arthritis | TNFR:Fc Fusion Gene | AAV2 |
| Leber Congenital Amaurosis | GAA | AAV1 |
| Limb Girdle Muscular Dystrophy Type | gamma-sarcoglycan | AAV1 |
| 2C\|Gamma-sarcoglycanopathy | | |
| Retinitis Pigmentosa | hMERTK | AAV2 |
| Age-Related Macular Degeneration | sFLT01 | AAV2 |
| Becker Muscular Dystrophy and | huFollistatin344 | AAV1 |
| Sporadic Inclusion Body Myositis | | |
| Parkinson's Disease | GDNF | AAV2 |
| Metachromatic Leukodystrophy (MLD) | cuARSA | AAVrh.10 |
| Hepatitis C | anti-HCV shRNA | AAV8 |
| Limb Girdle Muscular Dystrophy Type | hSGCA | AAVrh74* |
| 2D | | |
| Human Immunodeficiency Virus | PG9DP | AAV1 |
| Infections; HIV Infections (HIV-1) | | |
| Acute Intermittant Porphyria | PBGD | AAV5 |
| Leber's Hereditary Optical Neuropathy | P1ND4v2 | AAV2 |
| Alpha-1 Antitrypsin Deficiency | alpha1AT | AAVrh10 |
| Pompe Disease | hGAA | AAV9 |
| X-linked Retinoschisis | RS1 | AAV8 |
| Choroideremia | hCHM | AAV2 |
| Giant Axonal Neuropathy | JeT-GAN | AAV9 |
| Duchenne Muscular Dystrophy | rmicro-Dystrophin | AAVrh74* |
| X-linked Retinoschisis | hRS1 | AAV2 |
| Squamous Cell Head and Neck Cancer; | hAQP1 | AAV2 |
| Radiation Induced Xerostomia | | |
| Hemophilia B | Factor IX | AAVrh10/ |
| | | Rh74 |
| Homozygous FH | hLDLR | AAV8 |
| Dysferlinopathies | rAAVrh74.MHCK7.DYSF.DV | AAVrh74 |
| Hemophilia B | AAV6 ZFP nuclease | AAV6 |
| MPS I | AAV6 ZFP nuclease | AAV6 |
| Rheumatoid Arthritis | NF-kB.IFN-β | AAV5 |
| Batten/CLN6 | CLN6 | AAV9 |
| Sanfilippo Disease Type A | hSGSH | AAV9 |
| Osteoarthritis | 5IL-1Ra | AAV2.5 |
| Achromatopsia | CNGA3 | AAV2tYF |
| Achromatopsia | CNGB3 | AAV8 |
| Ornithine Transcarbamylase (OTC) | OTC | scAAV8 |
| Deficiency | | |
| Hemophilia A | Factor VIII | LK03/AAV3B |
| Mucopolysaccharidosis II | ZFP nuclease | AAV6 |
| Hemophilia A | ZFP nuclease | AAV6 |
| Wet AMD | anti-VEGF | AAV8 |
| X-Linked Retinitis Pigmentosa | PGR | AAV2 |
| Mucopolysaccharidosis Type VI | hARSB | AAV8 |
| Leber Hereditary Optic Neuropathy | ND4 | AAV2 |
| X-Linked Myotubular Myopathy | MTM1 | AAV8 |
| Crigler-Najjar Syndrome | UGT1A1 | AAV8 |
| Achromatopsia | CNGB3 | AAV8 |
| Retinitis Pigmentosa | hPDE6B | AAV5 |
| X-Linked Retinitis Pigmentosa | RPGR | AAV2tYF |
| Mucopolysaccharidosis Type 3 B | hNAGLU | AAV9 |
| Duchenne Muscular Dystrophy | GALGT2 | AAVrh74 |
| Arthritis, Rheumatoid; Arthritis, | TNFR:Fc Fusion Gene | AAV2 |
| Psoriatic; Ankylosing Spondylitis | | |
| Idiopathic Parkinson's Disease | Neurturin | AAV2 |
| Alzheimer's Disease | NGF | AAV2 |
| Human Immunodeficiency Virus | tgAAC09 | AAV2 |
| Infections; HIV Infections (HIV-1) | | |
| Familial Lipoprotein Lipase Deficiency | LPL | AAV1 |
| Idiopathic Parkinson's Disease | Neurturin | AAV2 |
| Alpha-1 Antitrypsin Deficiency | hAAT | AAV1 |
| Leber Congenital Amaurosis (LCA) 2 | hRPE65v2 | AAV2 |
| Batten Disease; Late Infantile | CLN2 | AAVrh.10 |
| Neuronal Lipofuscinosis | | |
| Parkinson's Disease | GAD | AAV2 |
| Sanfilippo Disease Type A/ | N-sulfoglucosamine | AAVrh.10 |
| Mucopolysaccharidosis Type IIIA | sulfohydrolase (SGSH) gene | |
| Congestive Heart Failure | SERC2a | AAV1 |
| Becker Muscular Dystrophy and | rAAV1.CMV.huFollistatin344 | AAV1 |
| Sporadic Inclusion Body Myositis | | |
| Parkinson's Disease | hAADC-2 | AAV2 |
| Choroideremia | REP1 | AAV2 |
| CEA Specific AAV-DC-CTL | CEA | AAV2 |
| Treatment in Stage IV Gastric Cancer | | |
| Gastric Cancer | MUC1-peptide-DC-CTL | |
| Leber's Hereditary Optical Neuropathy | scAAV2-P1ND4v2 | scAAV2 |
| Aromatic Amino Acid Decarboxylase | hAADC | AAV2 |
| Deficiency | | |
| Hemophilia B | Factor IX | AAVrh10 |
| Parkinson's Disease | AADC | AAV2 |
| Leber Hereditary Optic Neuropathy | Genetic: GS010\|Drug: Placebo | AAV2 |
| SMA - Spinal Muscular Atrophy\|Gene | SMN | AAV9 |
| Therapy | | |
| Hemophilia A | B-Domain Deleted Factor VIII | AAV8 |
| MPS I | IDUA | AAV9 |
| MPS II | IDS | AAV9 |
| CLN3-Related Neuronal Ceroid- | CLN3 | AAV9 |
| Lipofuscinosis (Batten) | | |
| Limb-Girdle Muscular Dystrophy, Type | hSGCB | rh74 |
| 2E | | |
| Alzheimer Disease | APOE2 | rh10 |
| Retinitis Pigmentosa | hMERKTK | AAV2 |
| Retinitis Pigmentosa | RLBP1 | AAV8 |
| Wet AMD | Anti-VEGF antibody | AAV2.7m8 |

For example, a rAAV vector comprising a transgene encoding glial derived growth factor (GDGF) finds use treating/preventing/managing Parkinson's disease. Generally, the rAAV vector is administered systemically. For example, the rAAV vector may be provided by intravenous, intrathecal, intra-nasal, and/or intra-peritoneal administration.

In certain embodiments, the transgene encodes a microdystrophin (for example, as disclosed in WO WO2021/108755, WO2002/029056, WO2016/115543, WO2015/197232, WO2016/177911, U.S. Pat. No. 7,892,824B2, U.S. Pat. No. 9,624,282B2, and WO2017221145, which are hereby incorporated by reference in their entirety) and is useful for treatment of dystrophinopathies, such as muscular dystrophy. Example 18 herein shows the relative abundance of capsids AAV7, AAV8, AAV9, AAVrh.10, AAVrh.46, AAVrh.64.R1, and AAVrh.73 after intravenous administration to wild-type mice compared to mdx mice (animal model for muscular dystrophy). rAAV particles having these capsids, or an engineered forms thereof, may be useful for delivery of transgenes encoding microdystrophins or other dystrophinopathy therapeutic proteins to muscle cells, including skeletal and/or cardiac muscle, while having reduced delivery to liver cells, for treatment of muscular dystrophies, such as, Duchenne Muscular Dystrophy.

In particular aspects, the rAAVs of the present invention find use in delivery to target tissues, or target cell types, including cell matrix associated with the target cell types, associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject tan rAAV where the peptide insertion is a homing peptide. In the case of Parkinson's, for example, a rAAV vector comprising a peptide insertion that directs the rAAV to neural tissue can be used, in particular, where the peptide insertion facilitates the rAAV in crossing the blood brain barrier to the CNS.

For a disease or disorder associated with neural tissue, an rAAV vector can be used that comprises a peptide insertion from a neural tissue-homing domain, such as any described herein. Diseases/disorders associated with neural tissue include Alzheimer's disease, amyotrophic lateral sclerosis (ALS), amyotrophic lateral sclerosis (ALS), Battens disease, Batten's Juvenile NCL form, Canavan disease, chronic pain, Friedreich's ataxia, glioblastoma multiforme, Huntington's disease, Late Infantile neuronal ceroid lipofuscinosis (LINCL), lysosomal storage disorders, Leber's congenital amaurosis, multiple sclerosis, Parkinson's disease, Pompe disease, Rett syndrome, spinal cord injury, spinal muscular atrophy (SMA), stroke, and traumatic brain injury. The vector further can contain a transgene for therapeutic/prophylactic benefit to a subject suffering from, or at risk of developing, the disease or disorder (see Tables 1A-1B).

The rAAV vectors of the invention also can facilitate delivery, in particular, targeted delivery, of oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. Also, the rAAV molecule of the invention may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once, or may be administered in a series of 2, 3 or 4 or more times, for example, weekly, monthly or every two months, 3 months, 6 months or one year until the series of doses has been administered.

The rAAV molecules of the invention may be administered alone or in combination with other prophylactic and/or therapeutic agents. Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In particular embodiments, such as where the transgene is intended to be expressed in the CNS, the vector is administered via lumbar puncture or via cisterna magna.

In certain embodiments, the agents of the invention are administered intravenously and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Intl. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e. g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, rAAVs can be used for in vivo delivery of transgenes for scientific studies such as optogenetics, gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In certain embodiments, the agent of the invention is substantially purified (i.e., substantially free from substances that limit its effect or produce undesired side-effects). In a specific embodiment, the host or subject is an animal, e.g., a mammal such as non-primate (e.g., cows, pigs, horses, cats, dogs, rats etc.) and a primate (e.g., monkey such as, a cynomolgus monkey and a human). In a certain embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

The following examples report an analysis of surface-exposed loops on the AAV9 capsid to identify candidates for capsid engineering via insertional mutagenesis. The invention is illustrated by way of examples, describing the construction of rAAV9 capsids engineered to contain 7-mer peptides designed on the basis of the human axonemal dynein heavy chain tail. Briefly, three criteria were used for selecting surface loops that might be amenable to short peptide insertions: 1) minimal side chain interactions with adjacent loops; 2) variable sequence and structure between serotypes (lack of conserved sequences); and 3) the potential for interrupting commonly targeted neutralizing antibody epitopes. A panel of peptide insertion mutants was constructed and the individual mutants were screened for viable capsid assembly, peptide surface exposure, and potency. The top candidates were then used as templates for insertion of homing peptides to test if these peptide insertion points could be used to re-target rAAV vectors to tissues of interest. Further examples, demonstrate the increased transduction and tissue tropism for certain of the modified AAV capsids described herein.

### 6.1. Example 1—Analysis of AAV9 Capsid

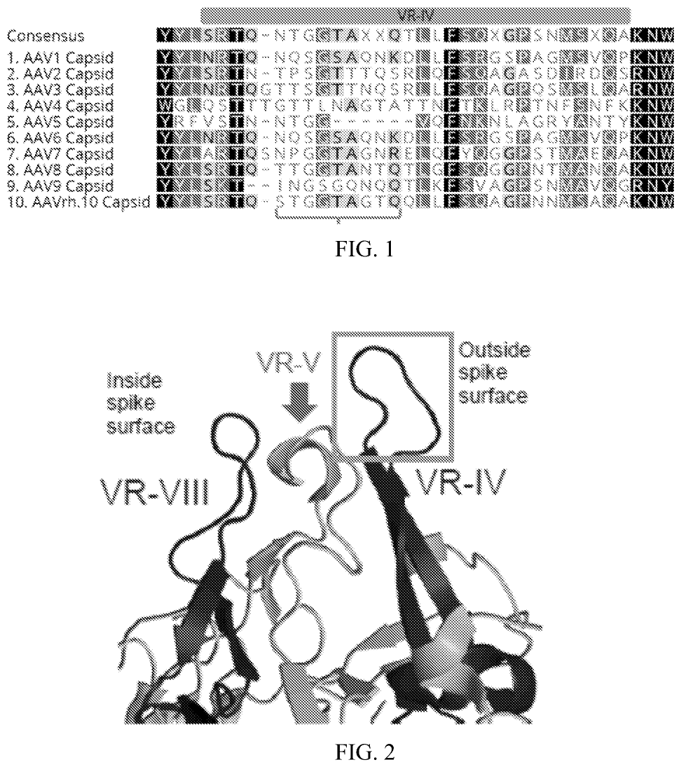

FIGS. 1 and 2 depict analysis of variable region four of the adeno-associated virus type 9 (AAV9 VR-IV) by amino acid sequence comparison to other AAVs VR-IV (FIG. 1) and protein model (FIG. 2). As seen, AAV9 VR-IV is exposed on the surface at the tip or outer surface of the 3-fold spike. Further analysis indicated that there are few side chain interactions between VR-IV and VR-V and that the sequence and structure of VR-IV is variable amongst AAV serotypes, and further that there is potential for interrupting a commonly-targeted neutralizing antibody epitope and thus, reducing immunogenicity of the modified capsid.

### 6.2. Example 2—Construction of AAV9 Mutants

Eight AAV9 mutants were constructed, to each include a heterologous peptide but at different insertion points in the VR-IV loop. The heterologous peptide was a FLAG tag that was inserted immediately following the following residues in vectors identified as pRGNX1090-1097, as shown in Table 2.

TABLE 2

| Vector designation | AAV9 VR-IV Insertion site for FLAG tag |
|---|---|
| pRGNX1090 | I451 |
| pRGNX1091 | N452 |
| pRGNX1092 | G453 |
| pRGNX1093 | S454 |
| pRGNX1094 | G455 |
| pRGNX1095 | Q456 |
| pRGNX1096 | N457 |
| pRGNX1097 | Q458 |

### 6.3. Example 3—Analysis of Packaging Efficiency

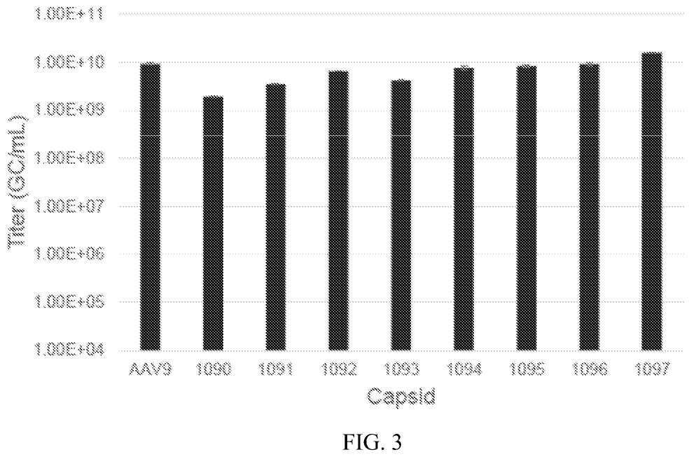

FIG. 3 depicts high packaging efficiency in terms of genome copies per mL (GC/mL) of wild type AAV9 and eight (8) candidate rAAV9 vectors (1090, 1091, 1092, 1093, 1094, 1095, 1096, and 1097), where the candidate vectors each contain a FLAG insert at different sites within AAV9's VR-IV. All vectors were packaged with luciferase transgene in 10 mL culture to facilitate determining which insertion points did not interrupt capsid packaging; error bars represent standard error of the mean.

As seen, all candidates package with high efficiency.

### 6.4. Example 4—Analysis of Surface FLAG Exposure

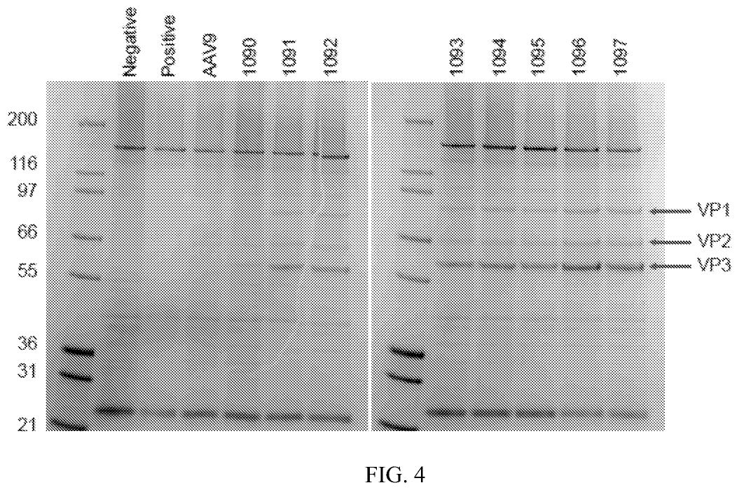

FIG. 4 depicts surface exposure of FLAG inserts in each of eight (8) candidate rAAV9 vectors (1090, 1091, 1092, 1093, 1094, 1095, 1096, and 1097), confirmed by immunoprecipitation of transduced vectors by binding to anti-FLAG resin. Binding to anti-FLAG indicates insertion points that allow formation of capsids that display the peptide insertion on the surface.

Transduced cells were lysed and centrifuged. 500 μL of cell culture supernatant was loaded on 20 μL agarose-FLAG beads and eluted with SDS-PAGE loading buffer also loaded directly on the gel. For a negative control, 293-ssc supernatant was used that contained no FLAG inserts.

As seen, 1090 had the lowest titer of the candidate vectors, indicating the least protein pulled down. Very low titers also were seen with the positive control. It is likely that not a sufficient amount of positive control had been loaded for visualization on SDS-PAGE.

### 6.5. Example 5—Analysis of Transduction Efficiency

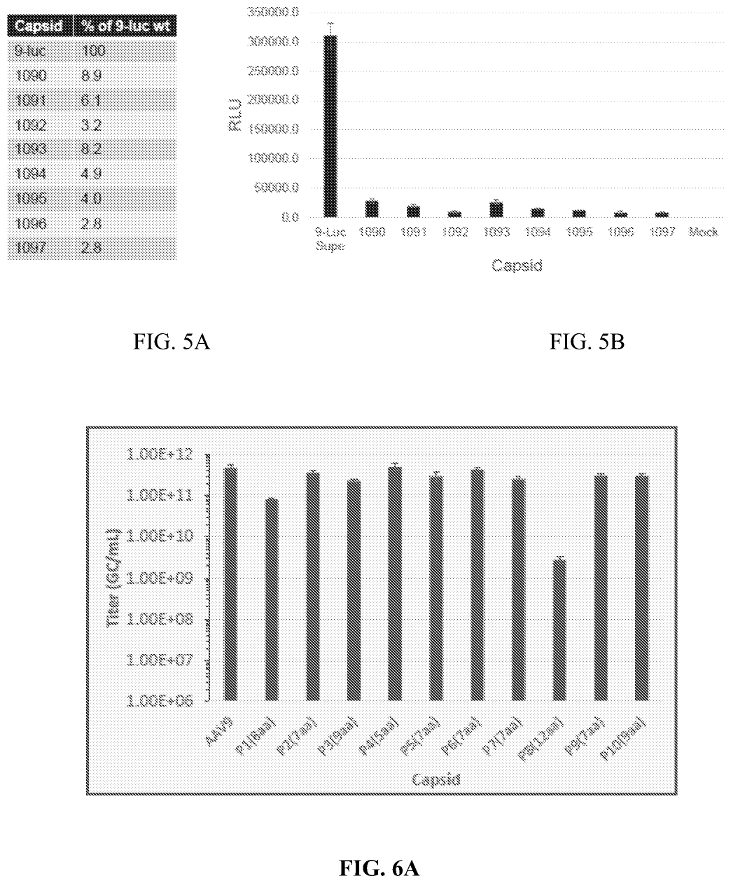

FIGS. 5A-5B depict transduction efficiency in Lec2 cells, transduced with capsid vectors carrying the luciferase gene as a transgene, that was packaged into either wild type AAV9 (9-luc), or into each of eight (8) candidate rAAV9 vectors (1090, 1091, 1092, 1093, 1094, 1095, 1096, and 1097); activity is expressed as percent luciferase activity, taking the activity of 9-luc as 100% (FIG. 5A), or as Relative Light Units (RLU) per microgram of protein (FIG. 5B).

CHO-derived Lec2 cells were grown in αMEM and 10% FBS. The Lec2 cells were transduced at a MOI of about 2×10⁸ GC vector (a MOI of about 10,000) and were treated with ViraDuctin reagent (similar results were observed on transducing Lec2 cells at a MOI of about 10,000 GC/cell but treated with 40 μg/mL zinc chloride (ZnC12); results not shown). Lec2 cells are proline auxotrophs from CHO.

As seen, transduction efficiency in vitro is lower than that obtained using wild type AAV9 (9-luc). Nonetheless, previous studies have shown that introduction of a homing peptide can decrease in vitro gene transfer in non-target cells (such as 293, Lec2, or HeLa), while significantly increasing in vitro gene transfer in target cells (see, e.g., Nicklin et al. 2001; and Grifman et al. 2001).

### 6.6. Example 6—Analysis of Packaging Efficiency as a Factor of Insertion Peptide Composition and Length

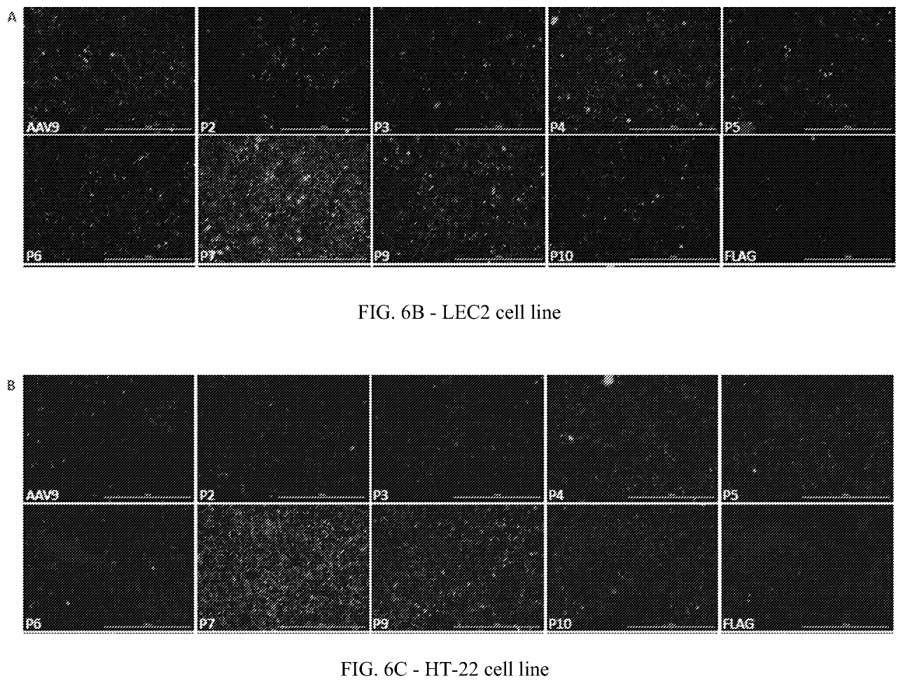

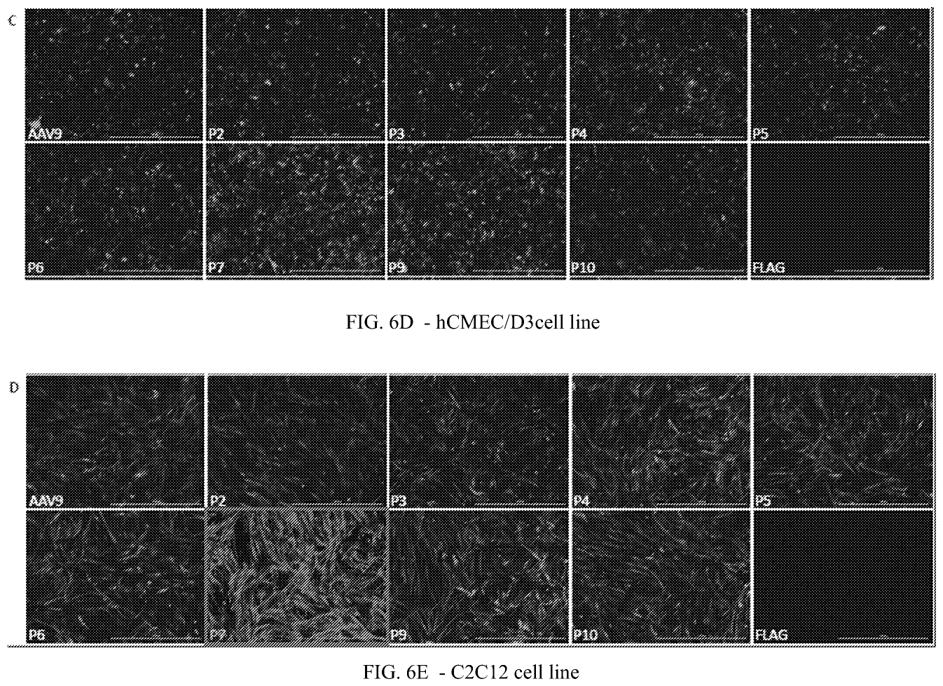

FIG. 6A depicts a bar graph illustrating that insertions immediately after 5454 of AAV9 capsid (SEQ ID NO:67) of varying peptide length and composition may affect production efficiencies of AAV particles in a packaging cell line. Ten peptides of varying composition and length were inserted after S454 (between residues 454 and 455) within AAV9 VR-IV. qPCR was performed on harvested supernatant of transfected suspension HEK293 cells five days post-transfection. The results depicted in the bar graph demonstrate that the nature and length of the insertions may affect the ability of AAV particles to be produced at high titer and packaged in 293 cells. (Error bars represent standard error of the mean length of peptide, which is noted on the Y-axis in parenthesis.)

AAV9 vectors having a capsid protein containing a homing peptide of the following peptide sequences (Table 3) at the S454 insertion site were studied. Suspension-adapted HEK293 cells were seeded at 1×10⁶ cells/mL one day before transduction in 10 mL of media. Triple plasmid DNA transfections were done with PEIpro® (Polypus transfection) at a DNA:PEI ratio of 1:1.75. Cells were spun down and supernatant harvested five days post-transfection and stored at −80° C.

TABLE 3

| Peptide | Tissue or Target | Peptide | SEQ ID |
|---|---|---|---|
| # | Designation | Sequence | NO: |
| P1 | Bone1 (D8) | DDDDDDDD | 2 |
| P2 | Brain1 | LSSRLDA | 3 |
| P3 | Brain2 | CLSSRLDAC | 4 |
| P4 | Kidney 1 | LPVAS | 6 |
| P5 | Kidney2 | CLPVASC | 5 |
| P6 | Muscle1 | ASSLNIA | 7 |
| P7 | TfR1 | HAIYPRH | 10 |
| P8 | TfR2 | THRPPMWS | 11 |
| | | PVWP | |
| P9 | TfR3 | RTIGPSV | 12 |
| P10 | TfR4 | CRTIGPSVC | 13 |

qPCR was performed on harvested supernatant of transfected suspension HEK293 cells five days post-transfection. Samples were subjected to DNase I treatment to remove residual plasmid or cellular DNA and then heat treated to inactivate DNase I and denature capsids. Samples were titered via qPCR using TaqMan Universal PCR Master Mix, No AmpEraseUNG (ThermoFisherScientific) and primer/probe against the polyA sequence packaged in the transgene construct. Standard curves were established using RGX-501 vector BDS.

Peptide insertions directly after 5454 ranging from 5 to 10 amino acids in length produced AAV particles having adequate titer, whereas an upper size limit is possible, with significant packaging deficiencies observed for the peptide insertion having a length of 12 amino acids.

### 6.7. Example 7—Homing Peptides Alter the Transduction Properties of AAV9 In Vitro when Inserted after S454

FIGS. 6B-E depict fluorescence images of cell cultures of (FIG. 6B) Lec2 cell line (sialic acid-deficient epithelial cell line) (FIG. 6C) HT-22 cell line (neuronal cell line), (FIG. 6D) hCMEC/D3 cell line (brain endothelial cell line), and (FIG. 6E) C2C12 cell line (muscle cell line). AAV9 wild type and S454 insertion homing peptide capsids of Table 3 containing GFP transgene were used to transduce the noted cell lines.

Cell lines were plated at 5-20×10³ cells/well (depending on the cell line) in 96-well 24 hours before transduction. Cells were transduced with AAV9-GFP vectors (with or without insertions) at 1×10¹⁰ particles/well and analyzed via Cytation5 (BioTek) 48-96 hours after transduction, depending on the difference in expression rate in each cell line. Lec2 cells were cultured as in Example 5, blood-brain barrier hCMEC/D3 (EMD Millipore) cells were cultured according to manufacturer's protocol, HT-22 and HU-17 cells were cultured in DMEM and 10% FBS, and C2C12 myoblasts were plated in DMEM and 10% FBS and differentiated for three days pre-transfection in DMEM supplemented with 2% horse serum and 0.1% insulin. AAV9.S454.FLAG showed low transduction levels in every cell type tested.

Images show that homing peptides can alter the transduction properties of AAV9 in vitro when inserted after S454 in the AAV9 capsid protein, as compared to unmodified AAV9 capsid. P7 (TfR1 peptide, HAIYPRH (SEQ ID NO:10)) for all cell lines show the highest rate of transduction followed by P9 (TfR3 peptide, RTIGPSV (SEQ ID NO:12)). P4 (Kidney1 peptide, LPVAS (SEQ ID NO:6)) showed a slightly higher rate of transduction than that of AAV9 wildtype for all cell types. Higher transduction rates were observed for P6 (Muscle1 peptide, ASSLNIA (SEQ ID NO:7)) in the brain endothelial hCMEC/D3 cell line and the C2C12 muscle cell line cultures as compared to the Lec2 and HT-22 cell line cultures. P1 vector was not included in images due to extremely low transduction efficiency, and P8 vector was not included due to low titer.

### 6.8. Example 8—Analysis of AAV Capsids for Peptide Insertion Points

FIG. 7 depicts alignment of AAVs 1-9e, rh10, rh20, rh39, rh74, hu12, hu21, hu26, hu37, hu51 and hu53 capsid sequences within insertion sites for capsid sequences within insertion sites for human peptides within or near the initiation codon of VP2, variable region 1 (VR-I), variable region 4 (VR-IV), and variable region 8 (VR-VIII) highlighted in grey; a particular insertion site within variable region eight (VR-VIII) of each capsid protein is shown by the symbol “#” (after amino acid residue 588 according to the amino acid numbering of AAV9).

### 6.9. Example 9—Comparison of AAV Genome Copies/in Genomic DNA of Various Vectors

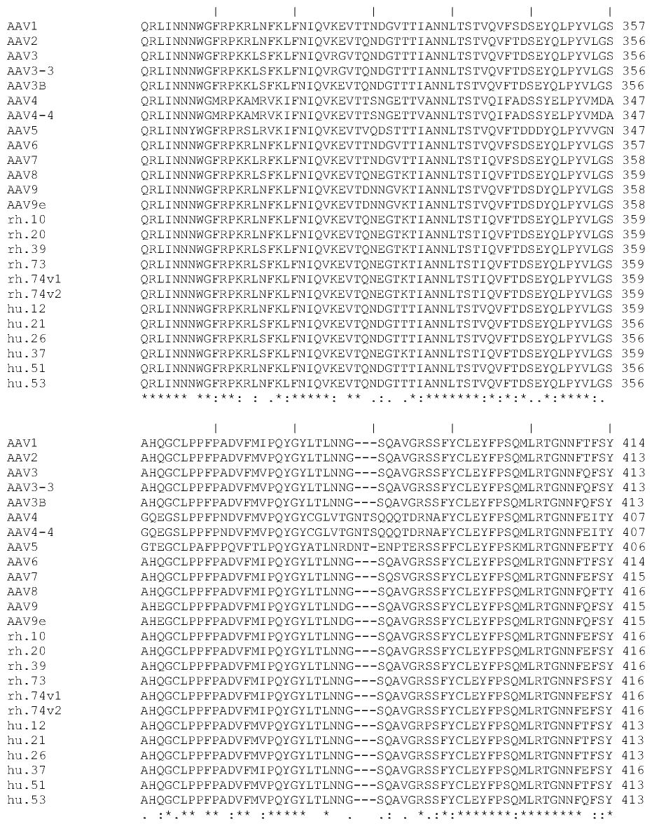

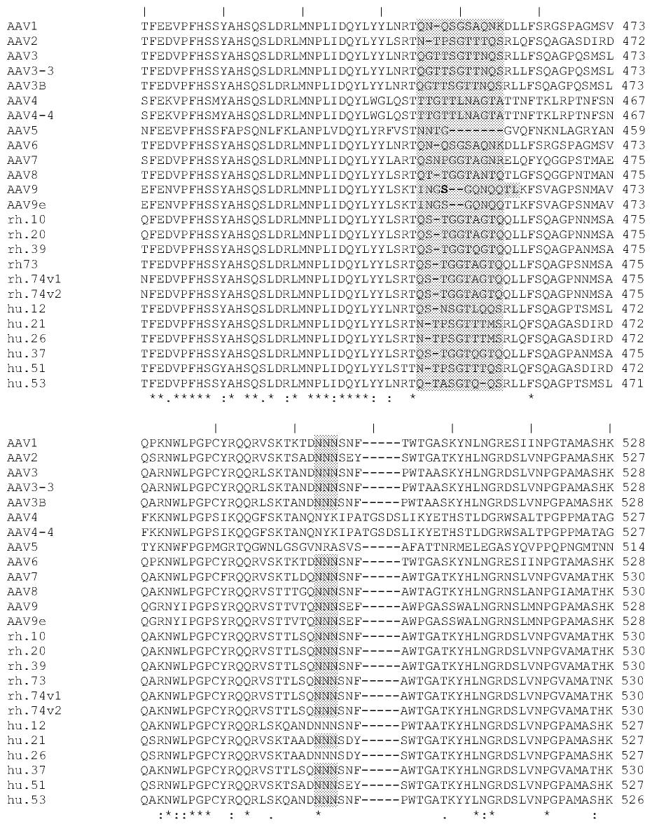

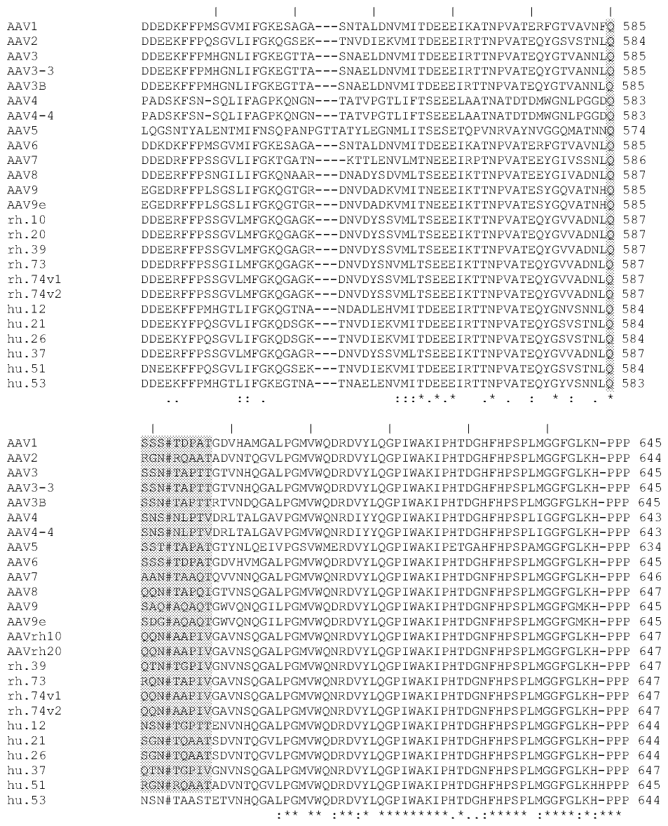

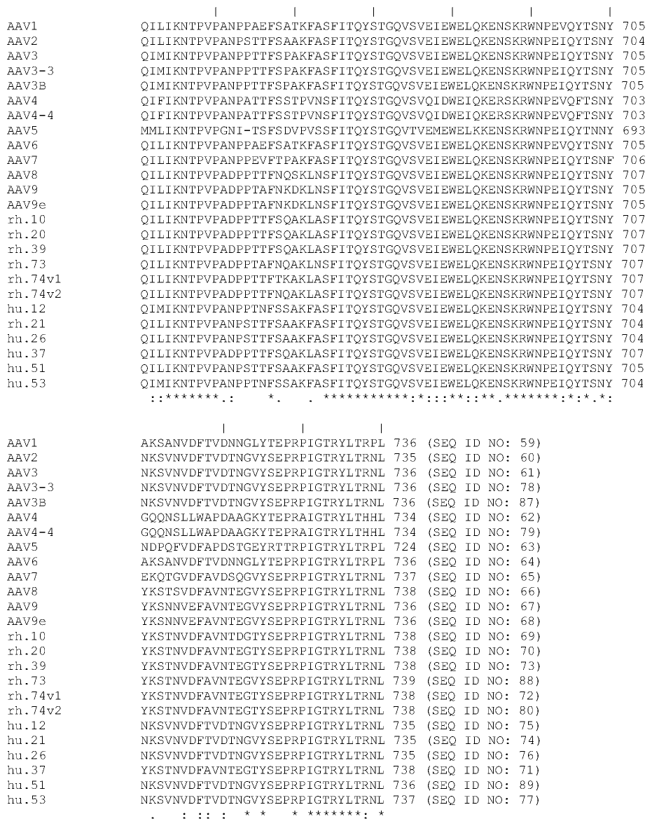

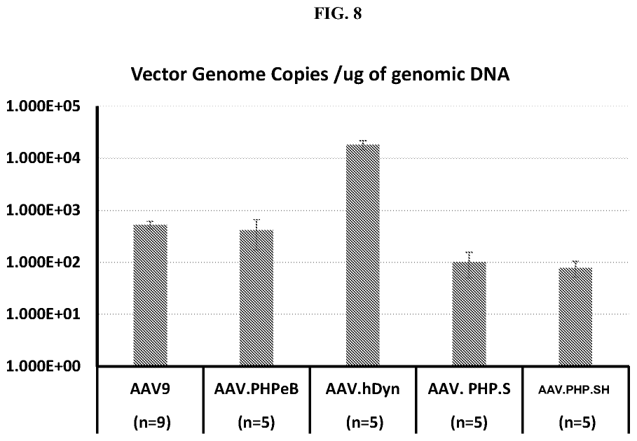

FIG. 8 depicts copies of GFP (green fluorescent protein) transgene expressed in mouse brain cells, following administration of the AAV vectors: AAV9; AAV.PHP.eB; AAV.hDyn (AAV9 with TLAAPFK (SEQ ID NO:1) between 588-589 with no other amino acid modifications to the capsid sequence); AAV.PHP.S; and AAV.PHP.SH (see Table 17).

AAV.PHP.B is a capsid having a TLAVPFK (SEQ ID NO:20) insertion in AAV9 capsid, with no other amino acid modifications to the capsid sequence. AAV.PHP.eB is a capsid having a TLAVPFK (SEQ ID NO:20) insertion in AAV9 capsid, with two amino acid modifications of the capsid sequence upstream of the PHP.B insertion (see also Table 17). Table 4A summarizes the capsids utilized in the study.

TABLE 4A

| | | | Location | | |
|---|---|---|---|---|---|
| | | | of | | SEQ |
| | Parent | | insertion | Peptide | ID |
| Name | capsid | Mutation | 2 | 2 | NO: |
| AAV9 | AAV9 | — | — | — | |
| PHP.B | AAV9 | | 588_589 | TLAVPFK | 20 |
| PHP.eB | AAV9 | 586A_587Q | 588_589 | TLAVPFK | 20 |
| | | delinsDG | | | |
| AAV. | AAV9 | — | 588_589 | TLAAPFK | 1 |
| hDyn | | | | | |
| AAV. | AAV9 | — | 588_589 | QAVRTSL | 16 |
| PHP.S | | | | | |
| AAV. | AAV9 | — | 588_589 | QAVRTSH | 17 |
| PHP.SH | | | | | |

### Materials and Methods

Constructs of AAV9, AAV.PHPeB, AAV.hDyn, AAV.PHP.S and AAV.PHP.SH encoding GFP transgene were prepared and formulated in 1×PBS+0.001% Pluronic. Female C57BL/6 mice were randomized into treatment groups base on Day 1 bodyweight. Five groups of female C57BL/6 mice were each intravenously administered AAV9.GFP, AAV.PHPeB.GFP, AAV.hDyn.GFP, AAV.PHP.S.GFP or AAV.PHP.SH.GFP in accordance with Table 4B, below. The dosing volume was 10 mL/kg (0.200 mL/20 g mouse). The mice were 8-12 weeks of age at the start date. At day 15 post administration, the animals were euthanized, and peripheral tissues were collected, including brain tissue, liver, forelimb biceps, heart, kidney, lung, ovaries, and the sciatic nerve.

TABLE 4B

| Gr. | N | Agent | Formulation dose | Route | Schedule |
|---|---|---|---|---|---|
| 1 | 9 | AAV9 | 2.5E12 GC/kg | iv | day 1 |
| 2 | 5 | PHPeB | 2.5E12 GC/kg | iv | day 1 |
| 3 | 5 | hDyn | 2.5E12 GC/kg | iv | day 1 |
| 4 | 5 | PHP.S | 2.5E12 GC/kg | iv | day 1 |
| 5 | 5 | PHP.SH | 2.5E12 GC/kg | iv | day 1 |

Quantitiative PCR (qPCR) was used to determine the number of vector genomes per μg of brain genomic DNA. Brain samples from injected mice were processed and genomic DNA was isolated using Blood and Tissue Genomic DNA kit from Qiagen. The qPCR assay was run on a QuantStudio 5 instrument (Life Technologies Inc) using primer-probe combination specific for eGFP following a standard curve method.

The AAV vector genome copies per μg of brain genomic DNA was at least a log higher in mice that were administered AAV.hDyn compared to all other AAV serotypes: AAV9, AAV. PHPeB, PHP. S, and PHP. SH (see FIG. 8). As seen in this study, GC/μg genomic DNA is highest for AAV.hDyn, which is AAV9 capsid containing the “TLAAPFK” (SEQ ID NO:1) peptide insert (a peptide from human axonemal dynein) between residues 588-589 of the AAV9 capsid. The study demonstrated transduction in mouse brain at greater than 1E04 GC/μg transgene on average in 5 mice systemically administered AAV.hDyn carrying eGFP. Other modified AAV9 capsids, however, including the vector AAV.PHPeB, which contains the “TLAVPFK” (SEQ ID NO:20) sequence (a peptide from mouse dynein) demonstrated transduction in mouse brain at less than 1E03 GC/μg transgene upon systemic treatment.

### 6.10. Example 10—Construction of rAAV Capsid Containing TLAAPFK (SEQ ID NO: 2)

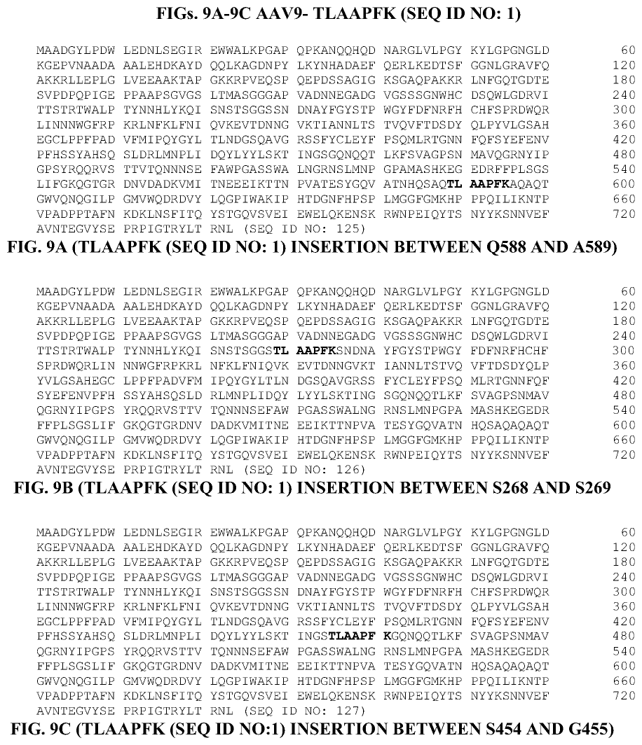

FIG. 9A depicts the amino acid sequence for a recombinant AAV9 vector capsid including a peptide insertion of amino acid sequence TLAAPFK (SEQ ID NO:1) between Q588 and A589 of VR-IIIV. Inserted peptide in bold.

FIG. 9B depicts the amino acid sequence for a recombinant AAV9 vector capsid including a peptide insertion of amino acid sequence TLAAPFK (SEQ ID NO:1) between S268 and S269 of VR-III. Inserted peptide in bold.

FIG. 9C depicts the amino acid sequence for a recombinant AAV9 vector capsid including a peptide insertion of amino acid sequence TLAAPFK (SEQ ID NO:1) between S454 and G455 of VR-IV. Inserted peptide in bold.

6.11. Example 11: Assessment of Modified Capsids In Vitro and In Vivo

AAV capsid sequences were modified either by peptide insertions or guided mutagenesis and pooled to give a bar-coded library packaged with a GFP expression cassette. The modified vectors were then evaluated in an in vitro assay, as well as for in vivo bio-distribution in mice using next generation sequencing (NGS) and quantitative PCR. AAV.hDyn was identified as a high brain transduction vector from this pool and was further evaluated in individual delivery studies in mice to characterize its transduction profile. Additionally, immunohistochemistry analysis of brain sections was performed to understand the cellular tropism of this vector.

### 6.11.1 Example 11A—In Vitro Testing of Transduction an Crossing Blood Brain Barrier

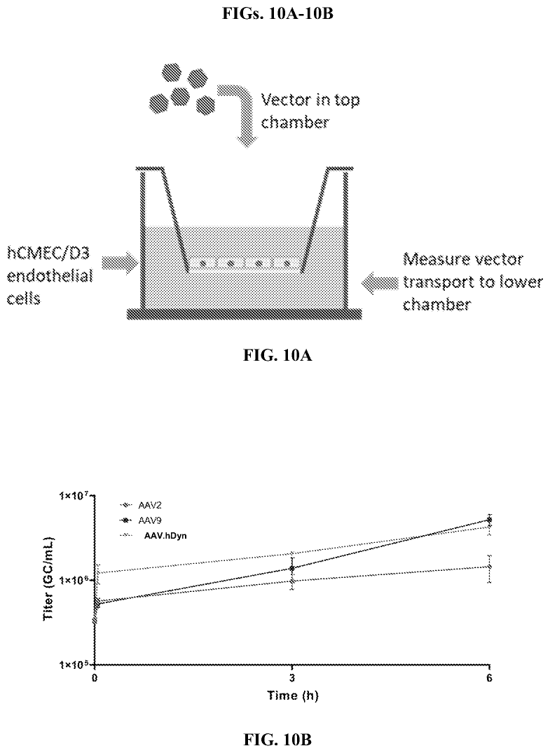

The ability of the modified capsids to cross the blood brain barrier was tested in an in vitro transwell assay using hCMEC/D3 BBB cells (SCC066, Millipore-Sigma) (see FIGS. 10A-10B). More specifically, the assay was essentially adapted from Sade, H. et al. (2014 PLoS ONE 9(4): e96340) A human Blood-Brain Barrier transcytosis assay reveals Antibody Transcytosis influenced by pH-dependent Receptor Binding, April 2014, Vol. 9, Issue 4; and Zhang, X., Blood-brain barrier shuttle peptides enhance AAV transduction in the brain after systemic administration, 2018 Biomaterials 176: 71-83. Briefly, 5×10⁴ hCMEC/D3 cells/cm² were seeded in collagen-coated transwell inserts in a 12-well plate. Each insert contained 500 μL media and the lower chamber contained 1 mL media. Media was replaced every second day. The supernatant was removed at 10 days post-seeding (the zero (0) time point). At this 0 time point, the cells were transduced by adding 1×10⁹ GC of vector to the upper insert chamber media. 104 lower chamber supernatant samples were removed for testing at intervals 0.5, 3, 6, and 23 hours post-transduction. Each condition (vector) was tested in duplicate, and measured for titer via qPCR against PolyA in triplicate.

FIGS. 10A-10B depict an in vitro transwell assay for AAV.hDyn (AAV9 with TLAAPFK (SEQ ID NO:1) between amino acid residues 588-589) crossing a blood brain barrier (BBB) cell layer (FIG. 10A), and results showing that AAV.hDyn (indicated by inverted triangles in the figure) crosses the BBB cell layer of the assay faster than AAV9 (squares), as well as faster and to a greater extent than AAV2 (circles) (FIG. 10B). The developed in vitro assay predicted enhanced BBB cross-trafficking and similar assays can be used to predict targeting to other organs as well.

### 6.11.2 Example 11B—Transduction and Biodistribution of Modified Capsids

6.11.2.1 Materials and Methods

Capsid modifications were performed on widely used AAV capsids including AAV8, AAV9, and AAVrh.10 by inserting various peptide sequences after the position 5454 of the VR-IV (Tables 5a-5c) or after position Q588 of the VR-VIII surface exposed loop of the AAV capsid, as well as insertions after the initiation codon of VP2, which begins at amino acid 137 (AAV4, AAV4-4, and AAV5) or at amino acid 138 (AAV1, AAV2, AAV3, AAV3-3, AAV6, AAV7, AAV8, AAV9, AAV9e, rh.10, rh.20, rh.39, rh.74, and hu.37) (FIG. 7) (see also Table 17 for certain capsid sequences). Selected single to multiple amino acid mutations were also used for modifying the capsids. See also, Yost et al., Structure-guided engineering of surface exposed loops on AAV Capsids. 2019. ASGCT Annual Meeting; and Wu et al., 2000 J. Virology (supra). It was confirmed that packaging efficiency was not negatively impacted following any of these capsid modifications in small scale.

rAAVs with certain modified capsids were tested for transduction in vitro in Lec2 cells as described above in Example 5. Modified AAVs tested for transduction in Lec2 cells as follows: eB 588 Ad, eB 588 Hep, eB 588 p79, eB 588 Rab, AAV9 588 Ad, AAV9 588 Hep, AAV9 588 p79, AAV9 588 Rab, eB VP2 Ad, eB VP2 Hep, eB VP2 p79, eB VP2 Rab, AAV9 VP2 Ad, AAV9 VP2 Hep, AAV9 VP2 p79, AAV9 VP2 Rab as compared to AAV9. See Table 5B below for identity of AAV capsids.

To test biodistribution, modified AAVs were packaged with an eGFP transgene cassette containing specific barcodes corresponding to each individual capsid. Novel barcoded vectors were pooled and injected into mice in order to increase the efficiency of screening.

To analyse the bio-distribution of genetically altered AAV vectors, various vectors encoding GFP were prepared and formulated in 1×PBS+0.0001% Pluronic acid. All vectors were made with cis plasmids containing a ten (10) bp barcode to enable next-generation sequencing (NGS) library (pool) preparation. Three (3) vector pools (Study 1, Study 2 and Study 3 vectors) were injected intravenously into a cohort of 5 female C57Bl/6 mice in accordance with Tables 5A-C. The dosing volume was 10 mL/kg (0.2 mL/20 g mouse) for each.

The mice were randomized into treatment groups based on Day 1 bodyweight and their age at start date was 8-12 weeks. At day 15 post administration, the animals were euthanized and peripheral tissues were collected, including brain, kidney, liver, sciatic nerve, lung, heart, and muscle tissue. In the studies where selected capsids from the pool were injected individually, the same protocol was followed

Genomic DNA (gDNA) was isolated from tissue samples using DNeasy Blood and Tissue kit (69506) from Qiagen. Each vector's barcode region was amplified with primers containing overlaps for NGS and unique dual indexing (UDI) and multiplex sequencing strategies, as recommended by the manufacturer (Illumina). Illumina MiSeq using reagent nano and micro kits v2 (MS-103-1001/1002) were used to determine the relative abundance of each barcoded AAV vector per sample collected from the mice. Accordingly, each vector sample in Tables 5A-C below was barcoded as noted above to allow for each read to be identified and sorted before the final data analysis. The data was normalized based on the composition of AAVs in the originally injected pool and quantified using the total genome copy number obtained from qPCR analysis with a primer-probe combination specific to the barcoded sample.

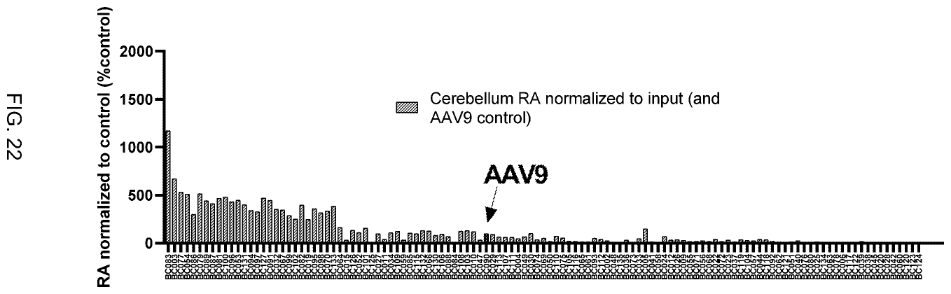

TABLE 5A

| | | | Insertion | | |
|---|---|---|---|---|---|
| Study 1 | Name | Capsid | Point | Peptide | Notes |
| BC01 | AAV9 | AAV9 | — | — | Blue bar, FIG. 21 |
| BC02 | PHP.eB | PHP.eB | 588_589 | TLAVPFK | |
| | | | | (SEQ ID | |
| | | | | NO: 20) | |
| BC03 | AAV8.BBB | Modified | — | — | A269S |
| | | AAV8 | | | |
| BC04 | AAV9.BBB | Modified | — | — | S263G/S269T/ |
| | | AAV9 | | | A273T |
| BC05 | AAV8.BBB.LD | Modified | — | — | A269S, 498- |
| | | AAV8 | | | NNN/AAA-500 |
| BC06 | AAV9.BBB.LD | Modified | — | — | S263G/S269T/A27 |
| | | AAV9 | | | 3T, 496-NNN/ |
| | | | | | AAA-498 |
| BC07 | rh. 10 | rh. 10 | — | — | |
| BC08 | rh. 10.LD | Modified rh. 10 | — | — | 498-NNN/ |
| | | | | | AAA-500 |
| BC09 | AAV.hDyn | modifiedAAV | 588_589 | TLAAPFK | Orange bar, |
| | | 9 | | (SEQ ID | FIG. 21 |
| | | | | NO: 1) | |
| BC10 | PHP.S | PHP.S | 588_589 | QAVRTSL | — |
| | | | | (SEQ ID | |
| | | | | NO: 16) | |
| BC11 | PHP.SH | PHP.SH | 588_589 | QAVRTSH | — |
| | | | | (SEQ ID | |
| | | | | NO: 17) | |
| BC13 | rh39 | rh.39 | — | — | — |

TABLE 5B

| | | | Insertion | | |
|---|---|---|---|---|---|
| Study 2 | Name | Capsid | Point | Peptide | Notes |
| BC20 | eB 588 Ad | PHP.eB | 588_589 | SITLVKSTQTV | Replaces |
| | | | | (SEQ ID NO: 14) | TLAVPFK peptide |
| | | | | | (SEQ ID NO: 20) |
| BC21 | eB 588 Hep | PHP.eB | 588_589 | TILSRSTQTG (SEQ | Replaces |
| | | | | ID NO: 15) | TLAVPFK peptide |
| | | | | | (SEQ ID NO: 20) |
| BC22 | eB 588 p79 | PHP.eB | 588_589 | VVMVGEKPITITQ | Replaces |
| | | | | HSVETEG (SEQ ID | TLAVPFK peptide |
| | | | | NO: 18) | (SEQ ID NO: 20) |
| BC23 | eB 588 Rab | PHP.eB | 588_589 | RSSEEDKSTQTT | Replaces |
| | | | | (SEQ ID NO: 19) | TLAVPFK peptide |
| | | | | | (SEQ ID NO: 20) |
| BC24 | 9 588 Ad | AAV9 | 588_589 | SITLVKSTQTV | |
| | | | | (SEQ ID NO: 14) | |
| BC25 | 9 588 Hep | AAV9 | 588_589 | TILSRSTQTG (SEQ | |
| | | | | ID NO: 15) | |
| BC26 | 9 588 p79 | AAV9 | 588_589 | VVMVGEKPITITQ | |
| | | | | HSVETEG (SEQ ID | |
| | | | | NO: 18) | |
| BC27 | 9 588 Rab | AAV9 | 588_589 | RSSEEDKSTQTT | |
| | | | | (SEQ ID NO: 19) | |
| BC28 | eB VP2 Ad | PHP.eB | 138_139 | SITLVKSTQTV | Also has |
| | | | | (SEQ ID NO: 14) | TLAVPFK (SEQ |
| | | | | | ID NO: 20) insert |
| | | | | | after residue 588 |
| BC29 | eB VP2 Hep | PHP.eB | 138_139 | TILSRSTQTG (SEQ | Also has |
| | | | | ID NO: 15) | TLAVPFK (SEQ |
| | | | | | ID NO: 20) insert |
| | | | | | after residue 588 |
| BC30 | eB VP2 p79 | PHP.eB | 138_139 | VVMVGEKPITITQ | Also has |
| | | | | HSVETEG (SEQ ID | TLAVPFK (SEQ |
| | | | | NO: 18) | ID NO: 20) insert |
| | | | | | after residue 588 |
| BC31 | AAV9 | AAV9 | — | — | |
| BC32 | eB VP2 Rab | PHP.eB | 138_139 | RSSEEDKSTQTT | Also has |
| | | | | (SEQ ID NO: 19) | TLAVPFK (SEQ |
| | | | | | ID NO: 20) insert |
| | | | | | after residue 588 |
| BC33 | 9 VP2 Ad | AAV9 | 138_139 | SITLVKSTQTV | |
| | | | | (SEQ ID NO: 14) | |
| BC34 | 9 VP2 Hep | AAV9 | 138_139 | TILSRSTQTG (SEQ | |
| | | | | ID NO: 15) | |
| BC35 | 9 VP2 p79 | AAV9 | 138_139 | VVMVGEKPITITQ | |
| | | | | HSVETEG (SEQ ID | |
| | | | | NO: 18) | |
| BC36 | 9 VP2 Rab | AAV9 | 138_139 | RSSEEDKSTQTT | |
| | | | | (SEQ ID NO: 19) | |

TABLE 5C

| | | | Insertion | | |
|---|---|---|---|---|---|
| Study 3 | Name | Capsid | Point | Peptide | Notes |
| BC01 | AAV9 | AAV9 | — | — | |
| BC03 | AAV8-BBB | AAV8 | — | — | A269S |
| BC07 | rh10 | rh. 10 | — | — | |
| BC09 | AAV.hDyn | AAV. | 588_589 | TLAAPFK | |
| | | hDyn | | (SEQ ID | |
| | | | | NO: 1) | |
| BC12 | PHP.B | PHP.B | 588_589 | TLAVPFK | |
| | | | | (SEQ ID | |
| | | | | NO: 20) | |
| BC20 | AAV9 S454- | AAV9 | 454_455 | DDDDDDDD | |
| | D8 | | | (SEQ ID | |
| | | | | NO: 2) | |
| BC22 | AAV9 S454- | AAV9 | 454_455 | LSSRLDA | |
| | Brain1 | | | (SEQ ID | |
| | | | | NO: 3) | |
| BC23 | AAV9 S454- | AAV9 | 454_455 | CLSSRLDAC | |
| | Brian 1C | | | (SEQ ID | |
| | | | | NO: 4) | |
| BC24 | AAV9 S454- | AAV9 | 454_455 | LPVAS | |
| | Kidney 1 | | | (SEQ ID | |
| | | | | NO: 6) | |
| BC25 | AAV9 S454- | AAV9 | 454_455 | CLPVASC | |
| | Kidney 1C | | | (SEQ ID | |
| | | | | NO: 5) | |
| BC26 | AAV9 S454- | AAV9 | 454_455 | ASSLNIA | |
| | Muscle1 | | | (SEQ ID | |
| | | | | NO: 7) | |
| BC27 | AAV9 S454- | AAV9 | 454_455 | HAIYPRH | |
| | TfR 1 | | | (SEQ ID | |
| | | | | NO: 10) | |
| BC29 | AAV9 S454- | AAV9 | 454_455 | RTIGPSV | |
| | TfR3 | | | (SEQ ID | |
| | | | | NO: 12) | |
| BC30 | AAV9 S454- | AAV9 | 454_455 | CRTIGPSVC | |
| | TfR4 | | | (SEQ ID | |
| | | | | NO: 13) | |
| BC31 | AAV9 S454- | AAV9 | 454_455 | DYKDDDDK | |
| | FLAG | | | (SEQ ID | |
| | | | | NO: 21) | |
| BC37 | pRGX1005- | PHP. | 588_589 | TLAVPFK | |
| | PHP.eB | eB | | (SEQ ID | |
| | (noBC) | | | NO: 20) | |

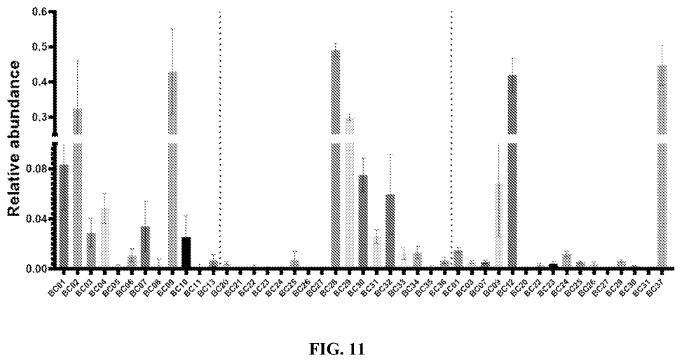

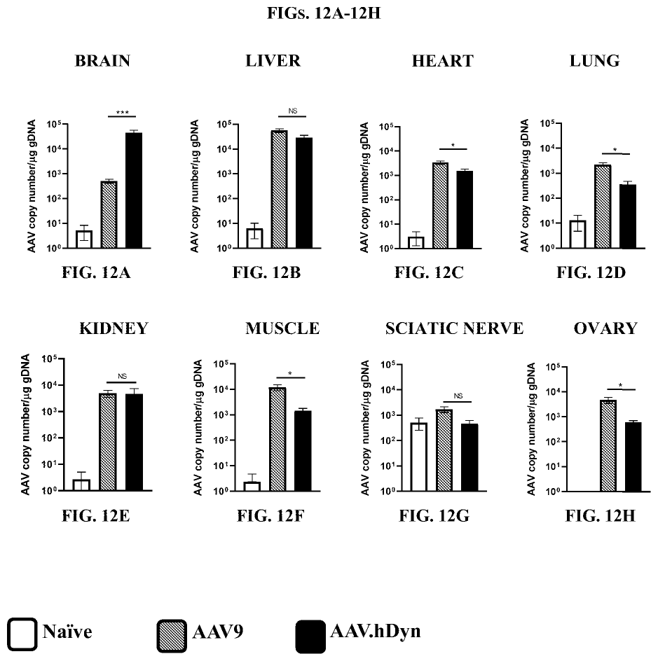

In the studies where selected capsids from the pool were injected individually, qPCR was used to determine the number of vector genomes per μg of tissue genomic DNA. qPCR was done on a QuantStudio 5 (Life Technologies, Inc.) using primer-probe combination specific for eGFP following a standard curve method (FIG. 12).

From the study where individual vectors were injected into mice for characterization, formalin fixed mouse brains were sectioned at 40 μm thickness on a vibrating blade microtome (VT1000S, Leica) and the floating sections were probed with antibodies against GFP to look at the cellular distribution of the delivered vectors.

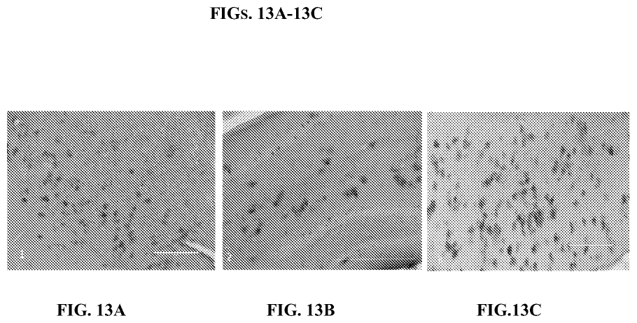

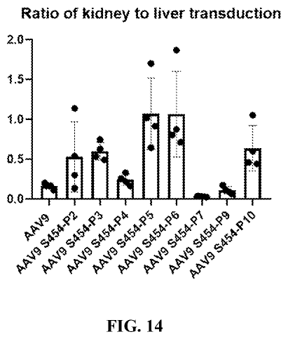

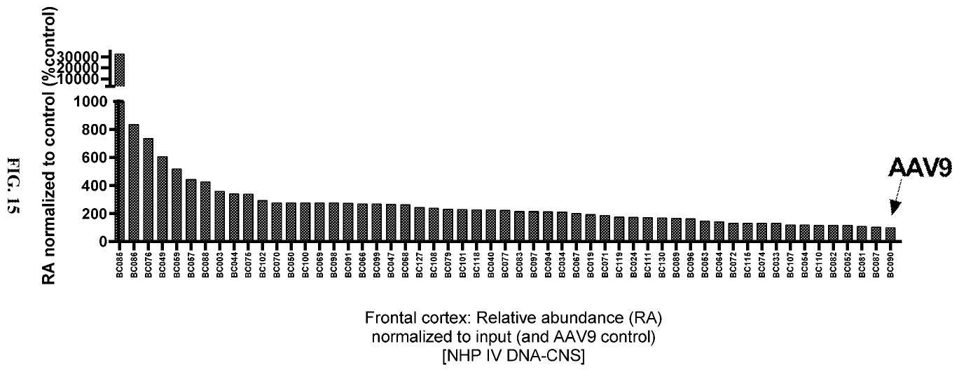

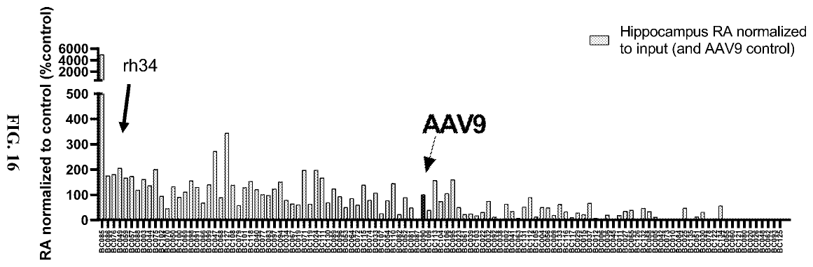

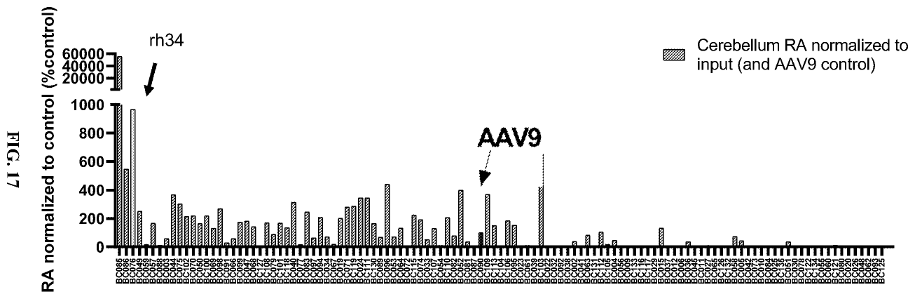

More specifically, fixed brains from the mice injected with AAV.hDyn were sectioned using a Vibratome (Leica, VT-1000) and the GFP expression was evaluated using an anti-GFP antibody (AB3080, Millipore Sigma), Vectastain ABC kit (PK-6100, Vector Labs) and DAB Peroxidase kit (SK-4100, Vector Labs). Broad distribution of GFP expressing cells were present throughout the brain in mice injected with AAV.hDyn, including distribution in the cortex, striatum, and hippocampus of the brain. FIGS. 13A-13C show the images from these regions and the scale bar is 400 um (discussed below).

6.11.2.2 Results

Results are shown in FIG. 11, FIGS. 12A-12H, and FIGS. 13A-13C.

Data for the Lec2 cell transduction assay not shown. The AAV9 588 Hep (AAV9 with the peptide TILSRSTQTG (SEQ ID NO:15) 5 inserted after position 588) exhibited significantly greater transduction (4-fold) than wild type AAV9, and AAV9 VP2 Ad (AAV9 with the peptide SITLVKSTQTV (SEQ ID NO:14) inserted after position 138), AAV9 VP2 Hep (AAV9 with the peptide TILSRSTQTG (SEQ ID NO:15) inserted after position 138), and AAV9 VP2 Rab (AAV9 with the peptide RSSEEDKSTQTT (SEQ ID NO:19) inserted after position 138) exhibited slightly greater transduction of the Lec2 cells relative to AAV9. The other AAVs assayed exhibited lower levels of transduction than AAV9.

FIG. 11 depicts results of Next Generation Sequencing (NGS) analysis of brain gDNA, revealing relative abundances (percent composition) of the capsid pool delivered to mouse brains following intravenous injection. The data was normalized based on the composition of AAVs in the originally injected pool and quantified using the total genome copy number obtained from qPCR analysis with a primer-probe combination specific to the eGFP sequence. Data shown are from three different experiments. Dotted lines indicate which vectors were pooled together. Parental AAV9 was used as standard and included in each pool. The “BC” identifiers are as indicated in Tables 5A, 5B and 5C above.

FIGS. 12A-12H depict an in vivo transduction profile of AAV.hDyn in female C57Bl/6 mice, showing copy number/microgram gDNA in naïve mice, or mice injected with either AAV9 or AAV.hDyn in brain (FIG. 12A), liver (FIG. 12B), heart (FIG. 12C), lung (FIG. 12D), kidney (FIG. 12E), skeletal muscle (FIG. 12F), sciatic nerve (FIG. 12G), and ovary (FIG. 12H), where AAV.hDyn shows increased brain bio-distribution compared to AAV9. The AAV vector genome copies per μg of brain genomic DNA was at least a log higher in mice that were administered AAV.hDyn compared to the parental AAV9 vector.

FIGS. 13A-13C show images from the regions analysed in the Immunohistochemical Analysis described above; scale bar is 400 μm. FIGS. 13A-13C depict distribution of GFP from AAV.hDyn throughout the brain, where images of immunohistochemical staining of brain sections from the striatum (FIG. 13A), hippocampus (FIG. 13B), and cortex (FIG. 13C) revealed a global transduction of the brain by the modified vector.

6.11.2.3 Conclusions

AAV capsid modifications performed either by insertions in surface exposed loops of VR-IV and VR-VIII or by specific amino acid mutations did not affect their packaging efficiency and were able to produce similar titers in the production system described herein.

Intravenous administration of AAV.hDyn to mice resulted in higher relative abundance of the viral genome and greater brain cell transduction than other modified AAV vectors and AAV9 tested.

TABLE 6
Homing peptides used in biodistribution study

| | | Location | | | |
|---|---|---|---|---|---|
| | | of | | | SEQ |
| | | Peptide | Peptide | Peptide | ID |
| Name | Capsid | Insertion | Name | Sequence | NO: |
| AAV9 | AAV9 | 454_455 | — | — | |
| AAV9 S454-P2 | AAV9 | 454_455 | Brain1 | LSSRLDA | 3 |
| AAV9 S454-P3 | AAV9 | 454_455 | Brain2 | CLSSRLDAC | 4 |
| | | | (Brain1C) | | |
| AAV9 S454-P4 | AAV9 | 454_455 | Kidney1 | LPVAS | 6 |
| AAV9 S454-P5 | AAV9 | 454_455 | Kidney2 | CLPVASC | 5 |
| | | | (Kidney | | |
| | | | 1C) | | |
| AAV9 S454-P6 | AAV9 | 454_455 | Muscle1 | ASSLNIA | 7 |
| AAV9 S454-P7 | AAV9 | 454_455 | Tfr1 | HAIYPRH | 10 |
| AAV9 S454-P9 | AAV9 | 454_455 | Tfr3 | RTIGPSV | 12 |
| AAV9 S454- | AAV9 | 454_455 | Tfr4 | CRTIGPSVC | 13 |
| P10 | | | | | |

AAV capsid modifications performed by insertions of different homing peptides in surface exposed loop VR-IV did not affect their packaging efficiency and were able to produce similar titers in the production system described herein.

Intravenous administration of AAV9 S454 Kidney1 and AAV9 S454 Kidney1C to mice resulted in higher relative abundance of the viral genome and greater kidney cell transduction than other modified AAV9 vectors and the parental AAV9 vector tested. Intravenous administration of the AAV9 S454 Kidney1 or AAV9 S454 Muscle1 vector to mice resulted also in lower liver cell transduction.

### 6.12. Example 12—Construction of rAAV Capsid Containing TLAVPFK (SEQ ID NO:20)

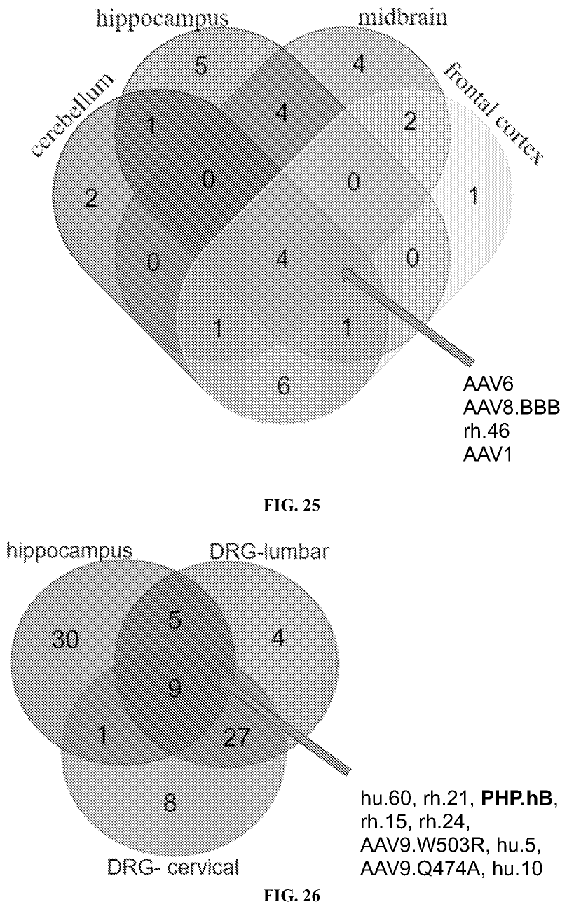

FIG. 25 depicts the amino acid sequence for a recombinant AAV9 vector capsid including a peptide insertion of amino acid sequence TLAVPFK (SEQ ID NO:20) between S454 and G455 of VR-IV.

### 6.13. Example 13—Biodistribution of an rAAV Vector Pool in Cynomolgus Monkeys

The administration, in vivo and post-mortem observations, and biodistribution of a pool of recombinant AAVs having engineered capsids and a GFP transgene will be evaluated following a single intravenous, intracerebroventricular or intravitreal injection in cynomolgus monkeys (Table 7). The pool contains multiple capsids each of which contains a unique barcode identification allowing identification using next generation sequencing (NGS) analysis following administration to cynomolgus monkeys. The cynomolgus monkey is chosen as the test system because of its established usefulness and acceptance as a model for AAV biodistribution studies in a large animal species and for further translation to human. All animals on this study are naïve with respect to prior treatment. The pool may comprise at least the following recombinant AAVs having the engineered capsids listed in Table 7.

TABLE 7
Recombinant AAVs for Cynomolgus monkey study

| | | | | | Peptide |
|---|---|---|---|---|---|
| | | Capsid | Location of | | NO: |
| Name | Capsid | modification | insertion | Peptide | SEQ ID |
| AAV8 | AAV8 | — | — | — | |
| AAV8.BBB | Modified | A269S | — | — | |
| | AAV8 | | | | |
| AAV8.BBB.LD | Modified | A269S, 498- | — | — | |
| | AAV8 | NNN/AAA-500 | | | |
| AAV9 | AAV9 | — | — | — | |
| AAV9 S454- | AAV9 | — | 454_455 | LSSRLDA | 3 |
| Brain1 | | | | | |
| AAV9 S454- | AAV9 | — | 454_455 | CLSSRLDAC | 4 |
| Brain1C | | | | | |
| AAV9 S454-D8 | AAV9 | — | 454_455 | DDDDDDDD | 2 |
| AAV9 S454- | AAV9 | — | 454_455 | LPVAS | 6 |
| Kidney 1 | | | | | |
| AAV9 S454- | AAV9 | — | 454_455 | CLPVASC | 5 |
| Kidney 1C | | | | | |
| AAV9 S454- | AAV9 | — | 454_455 | ASSLNIA | 7 |
| Muscle 1 | | | | | |
| AAV9 S454- | AAV9 | — | 454_455 | HAIYPRH | 10 |
| Tfr1 | | | | | |
| AAV9 S454- | AAV9 | — | 454_455 | RTIGPSV | 1219 |
| Tfr3 | | | | | |
| AAV9 S454- | AAV9 | — | 454_455 | CRTIGPSVC | 13 |
| TfR3C | | | | | |
| AAV9.496 | Modified | 498-NNN/AAA- | — | — | |
| NNN/AAA498 | AAV9 | 500 | | | |
| AAV9.496NNN/ | Modified | 498-NNN/AAA- | — | — | |
| AAA498.W503 | AAV9 | 500, W503R | | | |
| R | | | | | |
| AAV9.N272A.4 | Modified | N272A, | | | |
| 96NNN/AAA49 | AAV9 | 496-NNN/AAA- | | | |
| 8 | | 498 | | | |
| AAV9.G266A.4 | Modified | G266A, | | | |
| 96NNN/AAA49 | AAV9 | 496-NNN/AAA- | | | |
| 8 | | 498 | | | |
| AAV9.588Ad | AAV9 | — | 588_589 | SITLVKSTQ | 14 |
| | | | | TV | |
| AAV9.588Herp | AAV9 | — | 588_589 | TILSRSTQT | 15 |
| | | | | G | |
| AAV9.BBB | Modified | S263G/S269T/A27 | — | — | |
| | AAV9 | 3T | | | |
| AAV9.BBB.LD | Modified | S263G/S269T/A27 | — | — | |
| | AAV9 | 3T, 496- | | | |
| | | NNN/AAA-498 | | | |
| AAV9.Q474A | Modified | Q474A | | — | |
| | AAV9 | | | | |
| AAV9.W503R | Modified | W503R | — | — | |
| | AAV9 | | | | |
| AAVPHPeB.VP | PHP.eB | — | 138_139 | SITLVKSTQ | 14 |
| 2Ad | | | | TV | |
| AAVPHPeB.VP | PHP.eB | — | 138_139 | TILSRSTQT | 15 |
| 2Herp | | | | G | |
| PHP.B | AAV9 | — | 588_589 | TLAVPFK | 20 |
| PHP.eB | Modified | A587D, Q588G | 588_589 | TLAVPFK | 20 |
| | PHP.B | | | | |
| PHP.hB | | | | | |
| PHP.S | AAV9 | — | 588_589 | QAVRTSL | 16 |
| PHP.SH | AAV9 | — | 588_589 | QAVRTSH | 17 |

6.13.1. Study Design

Nine female cynomolgus animals will be used. Animals judged suitable for experimentation based on clinical sign data and prescreening antibody titers will be placed in study groups by body weight using computer-generated random numbers. Three different routes of administration will be used and relevant tissues collected to evaluate the biodistribution (measured by NGS and PCR) associated with the different routes. Three animals will be implanted with a catheter in the left lateral ventricle for intracerebroventricular (ICV) dose administration (Group 1), three animals will receive a single intravenous infusion (Group 2) and three animals will receive a single intravitreal injection (Group 3). Two animals will serve as replacement animals and will be implanted if required. Animals in Group 1 will have an MRI scan to determine coordinates for proper ICV catheter placement.

The IV infusion will be administered at a rate of 3 mL/min followed by 0.2 mL of vehicle to flush the dose from the IV catheter. The three intravenous animals will receive a single dose of the pooled recombinant AAVs at a volume of 4 mL/kg. The total dose (vg) and dose volume (mL/kg) will be recorded in the raw data. Based on literature review and previous studies in non-human primates, the IV dose of 1×10¹³ GC/kg body weight was determined to be required to have the desired distribution in the CNS from a systemic delivery as well as the peripheral tissues including skeletal muscle.

The ICV implanted animals will receive a single bolus dose at a volume of 1 mL of AAV-NAV-GFPbc (by slow infusion, approximately 0.1 mL/min) followed by 0.1 mL of vehicle to flush the dose from the catheter system. The ICV dose is based on distribution data from a previous non-human primate study to support current clinical programs.

The intravitreal (IVT) injection will be administered bilateral as a bolus injection at a dose volume of 50 μL.

6.13.2. Observations and Examinations

Clinical signs will be recorded at least once daily beginning approximately two weeks prior to initiation of dosing and continuing throughout the study period. The animals will be observed for signs of clinical effects, illness, and/or death. Additional observations may be recorded based upon the condition of the animal at the discretion of the Study Director and/or technicians.

Ophthalmological examinations will be performed on Group 3 animals prior to dose administration, and on Days 2, 8, 15 and 22. All animals will be sedated with ketamine hydrochloride IM for the ophthalmologic examinations performed following Day 1. For the examinations on Day 1, the animals will be sedated with injectable anesthesia (refer to Section 15.3.3). The eyes will be dilated with 1% tropicamide prior to the examination. The examination will include slit-lamp biomicroscopy and indirect ophthalmoscopy. Additionally, applanation tonometry will be performed on Group 3 animals prior to dosing, immediately following dose administration (˜10 to 15 minutes) and on Days 2 and 22.

Blood samples (˜3 mL) will be collected from a peripheral vein for neutralizing antibodies analysis approximately 2 to 3 weeks prior to dose administration.

6.13.3. Bioanalytical Sample Collection

Whole blood samples (˜0.5 mL) will be collected from a peripheral vein for bioanalytical analysis (AAV capsid clearance) prior to dose administration, 3 (±10 minutes), 6 (±10 minutes) and 24 (±0.5 hour) hours following dose administration from animals in Group 2 (IV) only. The samples will be collected using a syringe and needle, transferred to two K2 EDTA tubes and the times recorded.

Blood samples (˜5 mL) will be collected from fasted animals from a peripheral vein for PBMC analysis prior to dose administration (Day 1), on Days 8 and 15 and prior to necropsy (Day 22). The samples will be obtained using lithium heparin tubes and the times recorded.

Blood samples will be collected from a peripheral vein for bioanalytical analysis prior to dose administration (Day 1, 2 mL) and necropsy (Day 22, 5 mL). The samples will be collected in clot tubes and the times recorded. The tubes will be maintained at room temperature until fully clotted, then centrifuged at approximately 2400 rpm at room temperature for 15 minutes. The serum will be harvested, placed in labeled vials (necropsy sample split into 1 mL aliquots), frozen in liquid nitrogen, and stored at −60° C. or below.

CSF (˜1.5 mL) will be collected prior to dose administration from a cisterna magna spinal tap from animals in Group 1 only. CSF (˜2 mL) will be collected immediately prior to necropsy from a cisterna magna spinal tap from all animals (Groups 1 to 3). An attempt to collect CSF will be made but due to unsuccessful spinal taps, samples may not be collected at all intervals from an animal(s). Upon collection, the samples will be stored on ice until processing.

6.13.4. Necroscopy

A gross necropsy will be performed on any animal found dead or sacrificed moribund, and at the scheduled necropsy, following at least 21 days of treatment (Day 22). All animals, except those found dead, will be sedated with 8 mg/kg of ketamine HCl IM, maintained on an isoflurane/oxygen mixture and provided with an intravenous bolus of heparin sodium, 200 IU/kg. The animals will be perfused via the left cardiac ventricle with 0.001% sodium nitrite in saline. Animals found dead will be necropsied but will not be perfused.

The following tissues will be saved from all animals (including those found dead): Bone marrow, brain, cecum, colon, dorsal nerve roots and ganglion, duodenum, esophagus, eyes with optic nerves, gross lesions, heart, ileum, jejunum, kidneys, knee joint, liver, lungs with bronchi, lymph nodes, ovaries, pancreas, sciatic nerve, skeletal muscle, spinal cord, spleen, thyroids, trachea, and vagus nerve.

6.13.5. Bioanalytical Analysis

The whole blood collected from animals in Group 2 (IV) will be evaluated by qPCR and Next-Generation Sequencing (NGS).

PBMC samples collected from all animals will be evaluated by flow cytometry and enzyme-linked immune absorbent spot (ELISpot), if required.

The presence of circulating neutralizing antibodies as well as free vector in the serum and/or CSF will be evaluated by ELISA and cell based assays, as needed.

The vector copy number and number of transcripts in tissues will be examined by quantitative PCR and NGS methods.

### 6.14. Example 14—Study of CNS Biodistribution with Intravenous Administration

A procedure like that described in Example 13 was used to study the biodistribution of a pool of rAAV capsids administered intravenously to cynomolgus monkey model. Several capsids exhibited good spread in CNS with high relative abundance (RA, compared to AAV9 reference capsid) in most brain regions, notably AAV4, AAV5, rh 34, hu 26, rh31, and hu13. Favorable capsids exhibiting CNS-tropism have DNA RA values resulting in greater than 1.1-fold increase in DNA values in at least one CNS region, except dorsal root ganglion (DRG).

FIG. 15 depicts the relative abundance (RA) of the viral genomes (normalized to input) in the frontal cortex of the cynomolgus monkey model, and Table 8 lists the RA values for those capsids with the highest RA shown in FIG. 15.

TABLE 8

| Sample | | Relative | |
|---|---|---|---|
| No. | Serotype | Abundance | p-value |
| BC085 | AAV4 | 32643.06 | <0.0001 |
| BC086 | AAV5 | 836.64 | 0.0043 |
| BC076 | AAV.rh34 | 735.19 | 0.0296 |
| BC049 | AAV.hu26 | 606.37 | ns |
| BC059 | AAV.hu56 | 518.54 | ns |
| BC057 | AAV.hu53 | 444.2 | ns |
| BC088 | AAV7 | 427.28 | ns |
| BC003 | AAV8.BBB | 359.84 | ns |
| BC044 | AAV.hu13 | 341.58 | ns |
| BC075 | AAV.rh31 | 339.86 | ns |

FIGS. 16 and 17 depict the RA of the viral genomes (normalized to input) in the hippocampus and the cerebellum of the cynomolgus monkey model, respectively. The RA of AAV.rh34 is shown by the shaded column on the left side of the graphs and the RA of AAV9 reference is showed by the shaded column in the middle or the graphs. FIGS. 16 and 17 show that AAV.rh34 is a top performing capsid in the intravenous administration pool

AAV.rh34, displayed a favorable profile with respect to CNS toxicity as well. The rh34 capsid displayed decreased transduction in dorsal root ganglion (DRG) while exhibiting a high frontal cortex tropism (transduction efficiency). AAVrh34 exhibits an increased RA to AAV9 in CNS regions as follows: 1.8-fold in Hippocampus, 7.4-fold in frontal cortex, 1.9-fold in amygdala, 6.0-fold in medulla, 3.1-fold in midbrain, 1.2-fold in hypothalamus, 8.8-fold in thalamus, 13-fold in globus pallidus, 5.7-fold in SNc, 3.5-fold in dorsal raphe, 2.0-fold in claustrum, 13-fold in putamen, 9-fold in occipital cortex, and 9.6-fold in cerebellum. Additionally, AAVrh34 exhibits a decreased RA in: DRGs: 90-99.5%,Liver: ˜99%, Biceps: ˜30%, Sciatic nerve: 83%, and Optic nerve: 17%.

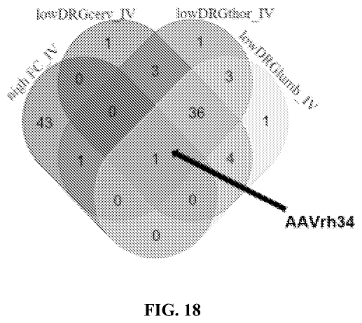

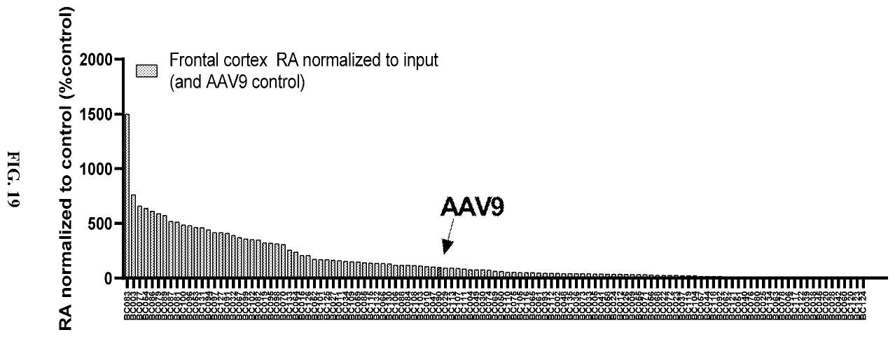

FIG. 18 depicts a Venn diagram of the: top 45 performers in FC (highest RA to AAV9), and bottom 45 performers in the cervical, thoracic, and lumbar DRGs (lowest RA to AAV9) and the AAVrh34 capsid is shown as the only capsid that was present in each group of 45 amongst the pool of capsids. AAV capsids with a combination of these recited characteristics are considered “DRG-friendly” capsids, such that their low rate of transduction in DRG should have minimal neurotoxicity and/or reduced or negligible axonopathy symptoms in a subject administered the AAV capsid.

### 6.15. Example 15: Study of CNS Biodistribution with Intracerebroventricular (ICV) Administration

A procedure like that described in Example 13 with ICV administration was used to study the biodistribution of a pool of rAAV capsids to cynomolgus monkey model. Several capsids exhibited good spread in CNS with high relative abundance (RA, compared to AAV9 reference capsid) in most brain regions, notably AAV4, AAV5, rh 34, hu 26, rh3l, and hu13. Favorable capsids exhibiting CNS-tropism have DNA relative abundance values resulting in greater than 1.1-fold increase in DNA values in at least one CNS region, except dorsal root ganglion (DRG).

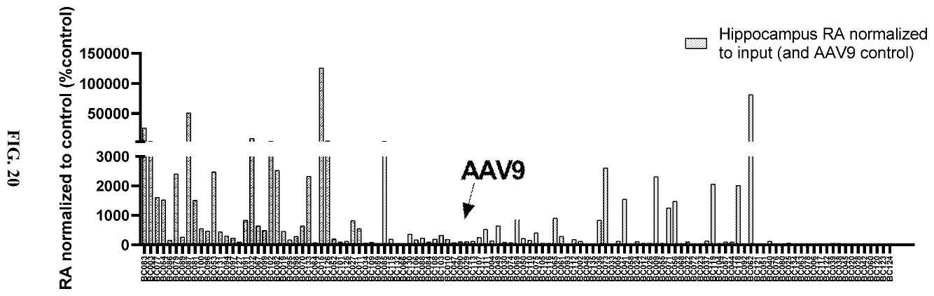

FIG. 19 depicts the RA of the viral genomes (normalized to input) in the frontal cortex of the cynomolgus monkey model, and Table 9 lists the RA values for those capsids with the highest RA shown in FIG. 19.

TABLE 9

| Sample | | | |
|---|---|---|---|
| No. | Serotype | RA | p-value |
| BC083 | AAV1 | 1499.86 | <0.0001 |
| BC003 | AAV8.BBB | 762.42 | 0.0151 |
| BC077 | AAV.rh4 | 659.09 | ns |
| BC054 | AAV.hu38 | 638.06 | ns |
| BC086 | AAV5 | 612.54 | ns |
| BC079 | AAV.rh46 | 588.72 | ns |
| BC089 | AAV8.BBB | 574.48 | ns |
| BC087 | AAV6 | 520.67 | ns |
| BC081 | AAV.rh72 | 512.76 | ns |

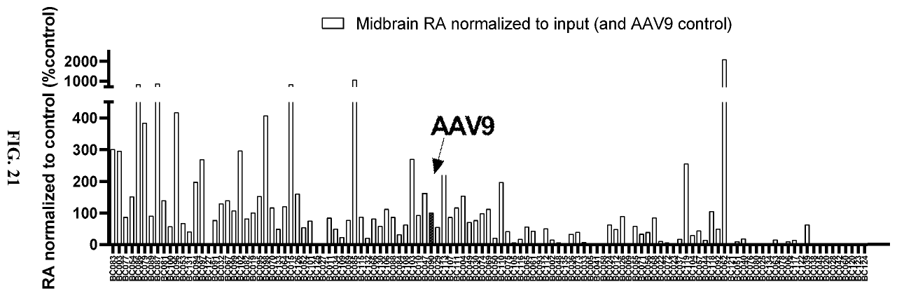

FIG. 20 depicts the relative abundance of the viral genomes (normalized to input) in the hippocampus of the cynomolgus monkey model, and Table 10 lists the RA values for those capsids with the highest RA shown in FIG. 20.

TABLE 10

| Sorted | Normalized Sorted | | |
|---|---|---|---|
| BC | Relative Abundance | Serotype | p-value |
| BC105 | 125921.21 | AAV2 | ns |
| BC062 | 81149.18 | AAV2 7m8 | ns |
| BC087 | 50962.40 | AAV6 | ns |
| BC083 | 25800.48 | AAV1 | ns |
| BC032 | 8113.37 | AAVrh2 | ns |
| BC126 | 4169.21 | AAVrh73 | ns |
| BC102 | 3673.77 | AAV8Y733F | ns |
| BC085 | 3397.77 | AAV4 | ns |
| BC003 | 3279.18 | AAV8.BBB | 0.007 |
| BC073 | 2609.49 | AAVrh24 | ns |

FIG. 21 depicts the relative abundance (RA) of the viral genomes (normalized to input) in the midbrain of the cynomolgus monkey model, and Table 11 lists the RA values for those capsids with the highest RA shown in FIG. 21.

TABLE 11

| Sorted | Normalized Sorted | | |
|---|---|---|---|
| BC | Relative Abundance | Serotype | p-value |
| BC062 | 2077.07 | AAV 7m8 | <0.0001 |
| BC085 | 1057.83 | AAV4 | 0.0002 |
| BC087 | 859.50 | AAV6 | 0.0104 |
| BC015 | 833.34 | AAV27m8 | 0.0164 |
| BC086 | 827.35 | AAV5 | 0.0181 |
| BC096 | 417.06 | AAV8 Y444F | 0.9781 |
| BC098 | 407.57 | AAV8 Y447F | ns |
| BC079 | 383.98 | AAVrh46 | ns |
| BC083 | 301.08 | AAV1 | ns |
| BC102 | 295.67 | AAV8 Y733F | ns |

FIG. 22 depicts the RA of the viral genomes (normalized to input) in the cerebellum of the cynomolgus monkey model, and Table 12 lists the RA values for those capsids with the highest RA shown in FIG. 22.

TABLE 12

| Sorted | Normalized | | |
|---|---|---|---|
| BC | Sorted RA | Serotype | p-value |
| BC083 | 1170.50 | AAV1 | <0.0001 |
| BC003 | 671.84 | AAV8.BBB | <0.0001 |
| BC077 | 531.50 | AAV rh4 | 0.0004 |
| BC079 | 515.31 | AAVrh46 | 0.0008 |
| BC054 | 510.51 | AAVhu38 | 0.001 |
| BC100 | 479.36 | AAV8Y707F | 0.0038 |
| BC127 | 470.57 | AAVrh2.R1.V651F | 0.0054 |
| BC081 | 467.79 | AAVrh72 | 0.006 |
| BC053 | 447.81 | AAVhu37 | 0.0128 |
| BC091 | 445.93 | AAV9Y6F | 0.0137 |

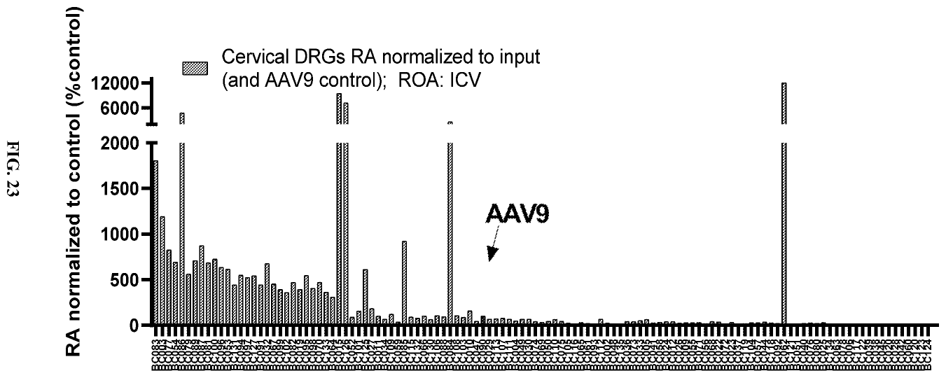

FIG. 23 depicts the RA of the viral genomes (normalized to input) in the cervical DRGs of the cynomolgus monkey model, and Table 13 lists the RA values for those capsids with the highest RA shown in FIG. 23.

TABLE 13

| Sorted | Normalized | | |
|---|---|---|---|
| BC | sorted RA | Serotype | p-value |
| BC062 | 12040.24 | AAV2 7m8 | <0.0001 |
| BC015 | 9522.20 | AAV2 | <0.0001 |
| BC126 | 7170.76 | AAVrh73 | ns (0.2422) |
| BC086 | 4760.90 | AAV5 | <0.0001 |
| BC084 | 2640.55 | AAV3B | ns |
| BC083 | 1808.40 | AAV1 | ns |
| BC003 | 1192.23 | AAV8.BBB | ns |
| BC085 | 922.02 | AAV4 | ns |
| BC087 | 875.60 | AAV6 | ns |
| BC077 | 826.27 | AAVrh4 | ns |

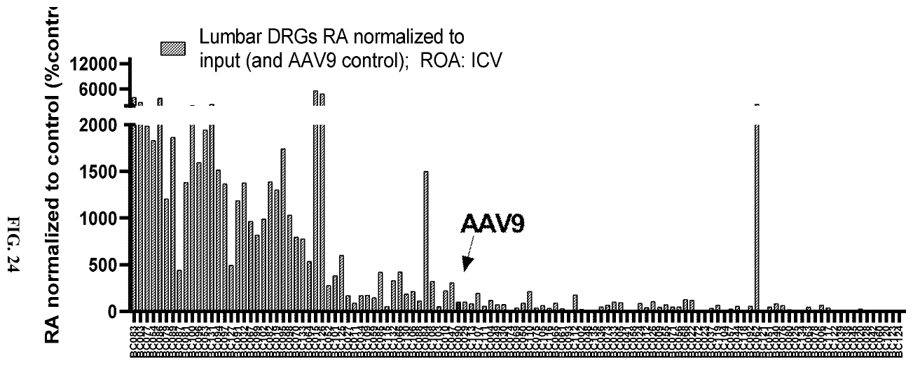

FIG. 24 depicts the RA of the viral genomes (normalized to input) in the lumbar DRGs of the cynomolgus monkey model, and Table 14 lists the RA values for those capsids with the highest RA shown in FIG. 24.

TABLE 14

| Sorted | Normalized | | |
|---|---|---|---|
| BC | sorted RA | Serotype | p-value |
| BC015 | 5649.13 | AAV2 | ns (0.9996) |
| BC126 | 4840.36 | AAVrh73 | <0.0001 |
| BC083 | 4002.82 | AAV1 | 0.0021 |
| BC086 | 3794.13 | AAV5 | 0.0051 |
| BC003 | 2847.53 | AAV8.BBB | ns |
| BC131 | 2427.59 | AAVrh64.R1 | ns |
| BC062 | 2367.34 | AAV2 7m8 | ns |
| BC100 | 2000.82 | AAV8 Y707F | ns |
| BC077 | 1988.30 | AAV rh4 | ns |
| BC053 | 1947.15 | AAVhu37 | ns |

FIG. 25 depicts a Venn diagram of the top performing 15 capsids transducing the frontal cortex, hippocampus, midbrain and cerebellum following ICV administration. As indicated in the diagram, AAV6, AAV8.BBB, AAV.rh.46, and AAV1 were the only AAVs represented in each of the top performing groups.

FIG. 26 depicts a Venn diagram of the top performing 45 capsids transducing the hippocampus and the 45 capsids with the lowest transduction values for DRG, to identify hippocampus-targeting DRG friendly capsids. As indicated in the diagram, AAV.hu.60, AAV.rh.21, AAV.PHP.hB, AAV.rh.15, AAV.rh.24, AAV9.W503R, hu.5, AAV9.Q474A, and AAV.hu.10 were the only AAVs represented in each of the groups.

### 6.16. Example 16—Study of Muscle and Liver Biodistribution with Intravenous (IV) Administration

A procedure like that described in Example 13 with IV administration was used to study the muscle and liver biodistribution of a pool of rAAV capsids to cynomolgus monkey model.

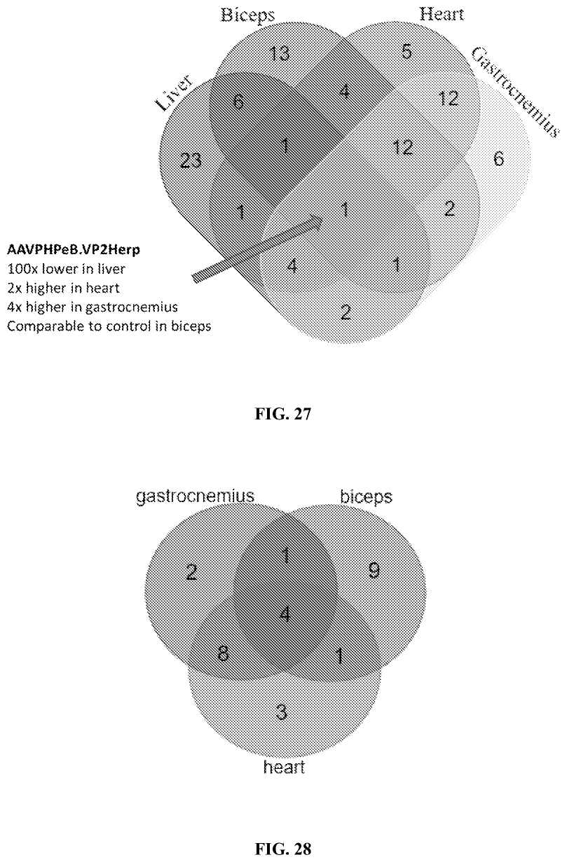

FIG. 27 depicts a Venn diagram of the top performing 40 capsids transducing the heart, biceps, and gastrocnemius and the 40 capsids with the lowest transduction values for the liver, to identify muscle-targeting liver-friendly capsids. As indicated in the diagram, AAV.PHPeB.VP2Herp was the only AAVs represented in each of the groups.

FIG. 28 depicts a Venn diagram of the top performing 15 capsids transducing the heart, biceps, and gastrocnemius and Table 15 provides a list of the top performing capsids in three different cells of the diagram.

TABLE 15

| Diagram Cell | Total | Serotype |
|---|---|---|
| biceps, gastrocnemius, heart | 4 | Hu.10, AAV3B, AAV2.7m8, AAV9.Q474A |
| biceps, gastrocnemius, | 1 | AAV5 |
| heart | 3 | Rh.15, PHP.hB, modified AAV8 |

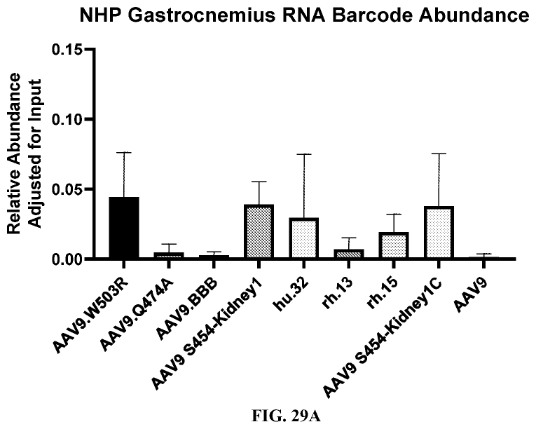

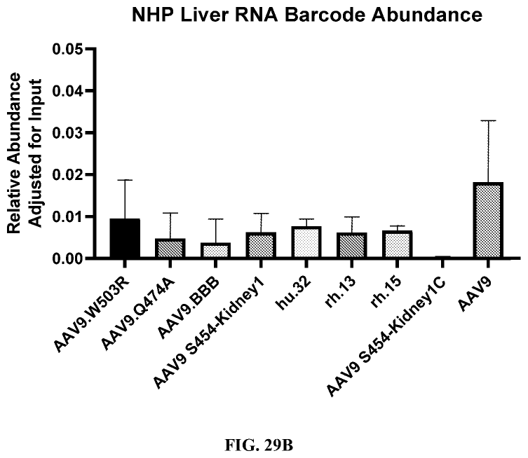

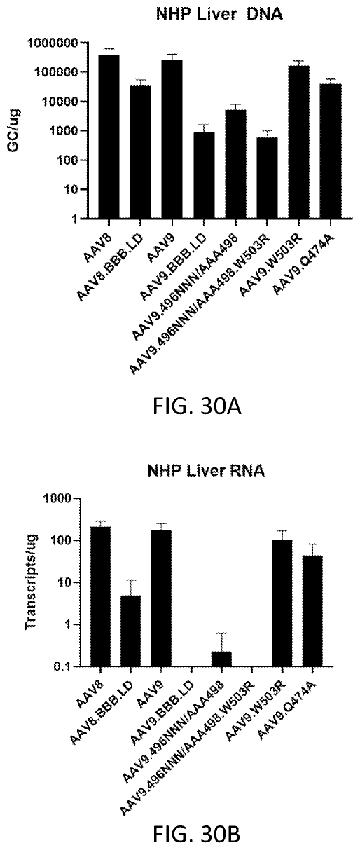

FIGS. 29A and B depict the RA of the viral genomes (normalized to input) in the gastrocnemius and the liver of the cynomolgus monkey model, respectively.

Table 16 provides the rank of each capsid by RA values for the cynomolgus monkey model and the MDX mouse model. Capsids were ranked relative to one another in each animal to decrease variability across animals. Gastrocnemius, TA, heart, bicep, and triceps contributed 70% to the ranking for the MDX Mouse Model and the gastrocnemius, heart, and biceps contributed 70% to the ranking for the cynomolgus monkey model. Liver RA contributed 30% to rankings for each animal. The overall ranking was determined by weighting the ranking for each animal 50%.

TABLE 16

| | NH/C. | | |
|---|---|---|---|
| | Monkey | MDX | Overall |
| Serotype | Rank | Rank | Rank |
| AAV9.W503R | 3 | 1 | 1 |
| AAV9.Q474A | 4 | 10 | 3 |
| AAV9.BBB | 15 | 2 | 4 |
| AAV9 S454- | 2 | 21 | 7 |
| Kidney 1 | | | |
| AAV.hu32 | 7 | 17 | 9 |
| AAV.rh13 | 24 | 5 | 12 |
| AAV.rh15 | 8 | 25 | 13 |
| AAV9 S454- | 5 | 31 | 17 |
| Kidney 1C | | | |
| AAV9 | 36 | 6 | 18 |

### 6.17. Example 17: Study of Liver Detargeting Biodistribution Following Intravenous Administration of Capsid Library

Pooled barcoded vectors were administered to NHPs by IV injection. The pooled mixture consists of 118 different AAV capsids, including natural isolates and engineered AAVs, as described herein, expressing the GFP reporter gene from the universal CAG promoter. The intravenous study followed the protocol described in Examples 13 and 14, infra. Several capsids exhibited tropism that “detargeted” the liver, as such, mutated capsids exhibited lower abundance in liver tissue than the parental capsid (AAV9), e.g. AAV8.BBB.LD (A269S, 498-NNN/AAA-500), AAV9.BBB.LD (S263G/S269T/A273T, 496-NNN/AAA-498), AAV9.496-NNN-498, AAV9.496-NNN-498.W503R, AAV9.W503R, and AAV9.Q474A. AAV8 capsids having the NNN/AAA mutation exhibit overall approximately an 11-fold reduction in transduction in liver, and 42-fold reduction in expression of transcript in liver. AAV9 capsids having the NNN/AAA and W503R mutation exhibits approximately a 400-fold reduction in transduction in liver, and results in zero expression of transcript in liver. In some instances, brain distribution of these modified vectors was also diminished. AAV8.BBB.LD additionally exhibits a high level of transduction in gastrocnemius muscle.

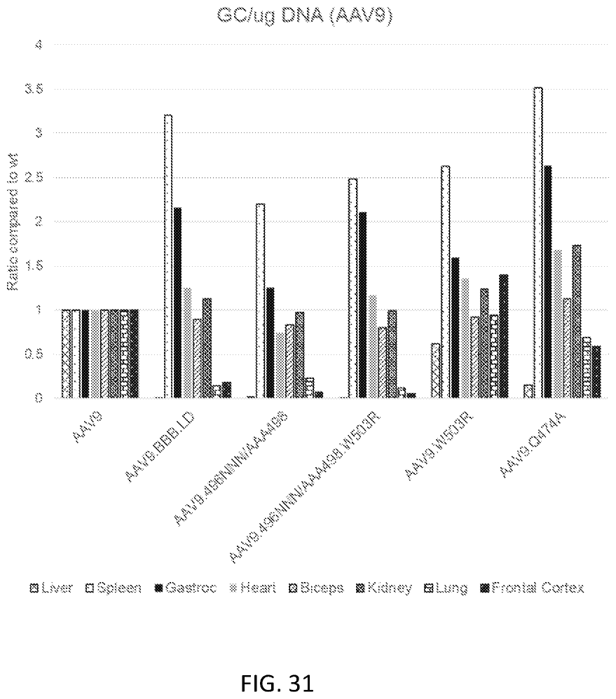

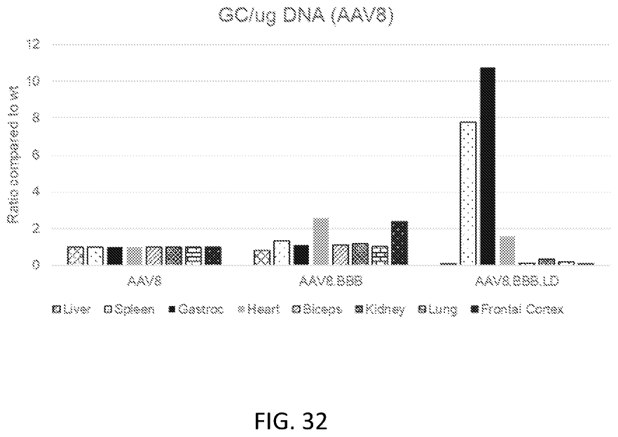

FIG. 31 depicts the biodistribution of select “liver-detargeting” (LD) vectors compared to their parental AAV9 capsid in various tissues, in NHPs following IV administration of the capsid library. FIG. 32 depicts the biodistribution of select LD vectors compared to their parental AAV8 capsid in various tissues, in NHPs following IV administration of the capsid library.

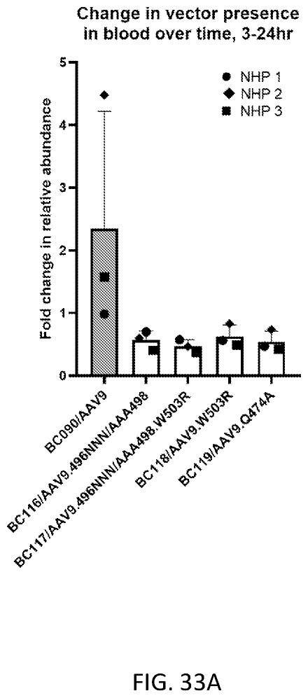

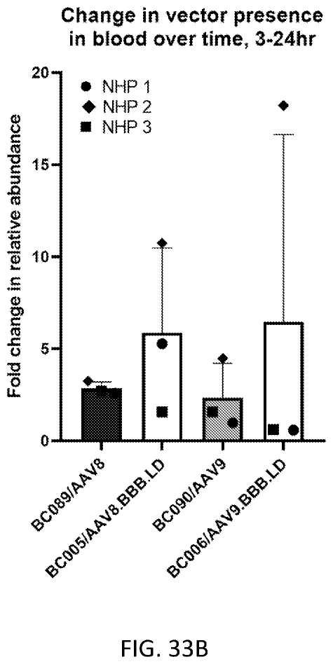

Studies also show the change in relative abundance (adjusted for input, normalized to 1) between the abundance for each barcode (and therefore capsid) at 3 hr post and 24 hr post IV capsid library administration (RA at 3 hr/RA at 24 hr). Individual animals are indicated by different shape data points (3 animals total) (FIGS. 33A and 33B).

A fold change >1 indicates that the capsid makes up a lower percentage of the total capsid “pool” present in the blood at 24 hr compared to 3 hr after dosing (i.e. faster blood clearance). A fold change <1 indicates that the capsid makes up a greater percentage of the total capsid “pool” present in the blood at 24 hr compared to 3 hr after dosing (i.e. slower clearance). Historically, slower clearance correlates with lower liver transduction/liver detargeting.

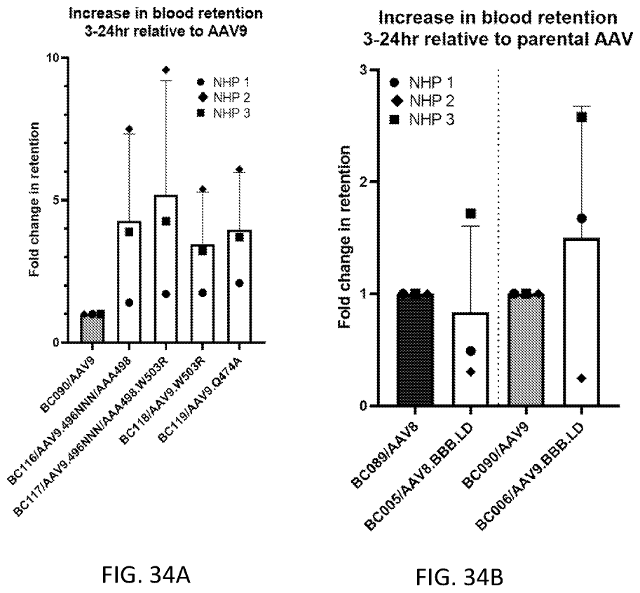

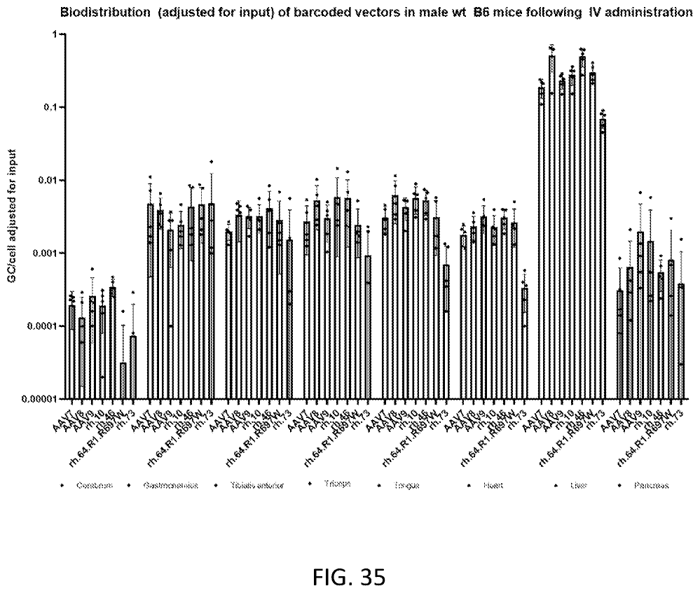

As represented by increase in blood retention, a depiction of the change in abundance for a given capsid in a given animal was plotted. Allowing for the calculation of the fold increase in blood retention over the baseline retention of AAV9, for example, the representations (FIGS. 34A and 34B) compare that change in abundance value of the select capsid to the change in abundance of the parental capsid (setting the parental capsid to 1). Thus, various mutations to the AAV9 capsid increase retention in the circulation by 3 to 5 fold (see. e.g. FIG. 34A).

### 6.18 Example 18—Study of Muscle, Liver and Brain Biodistribution with Intravenous (IV) Administration of Capsid Pool to mdx mice

Pooled barcoded vectors were administered to mdx mice by IV (tail vein) injection. The pooled mixture consists of 118 different AAV capsids, including natural isolates and engineered AAVs, as described herein, expressing the GFP reporter gene from the universal CAG promoter. The IV study followed a protocol analogous to that described in Examples 12 and 16, infra.

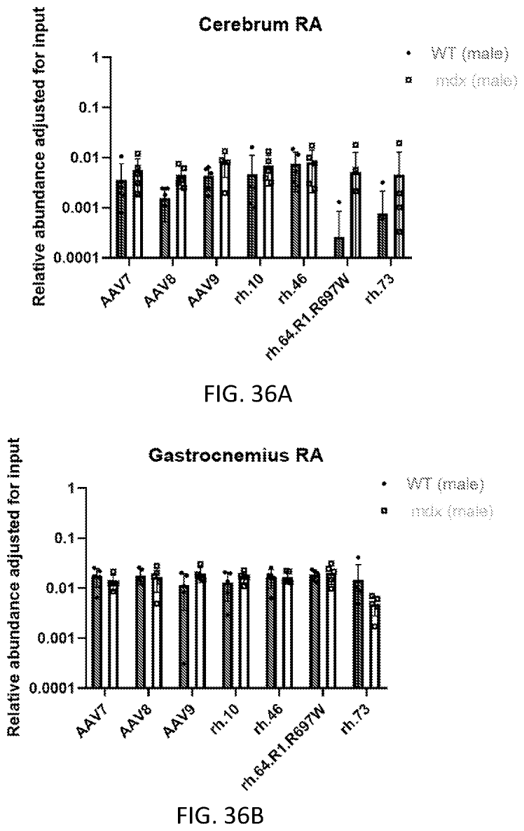

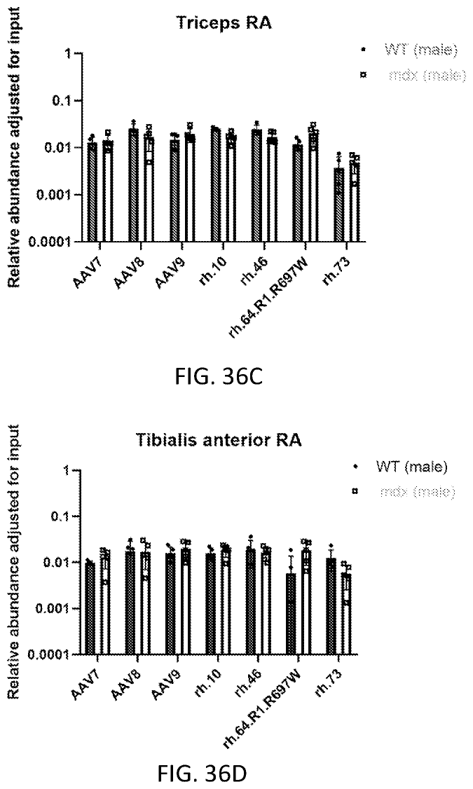

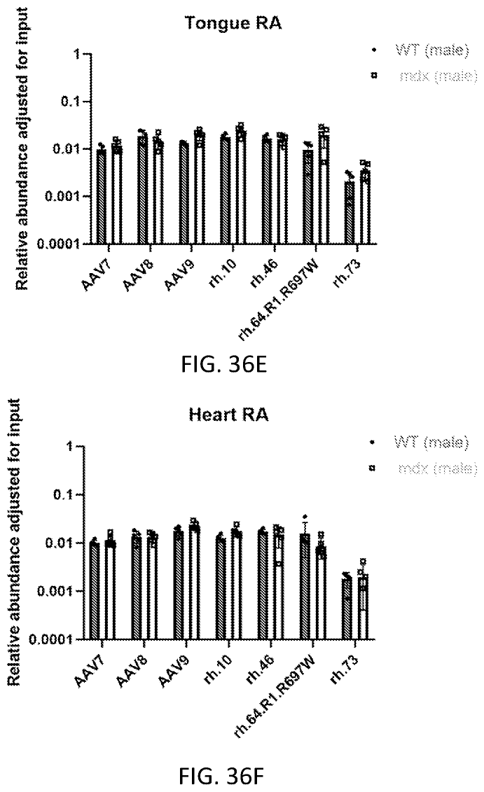

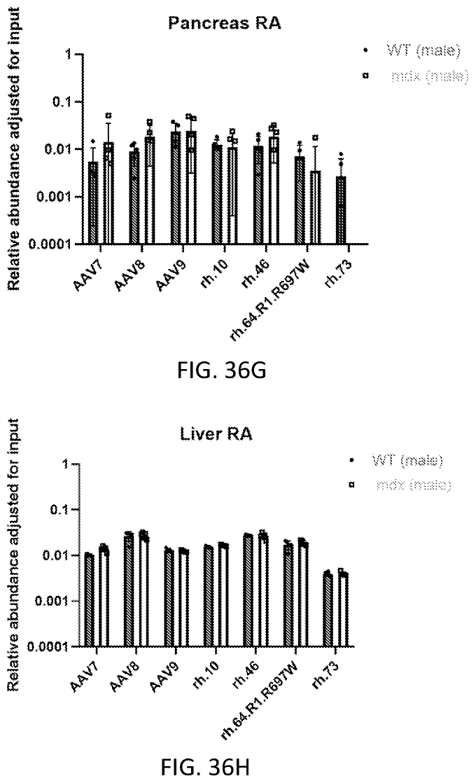

At 3 week sacrifice, tissues were harvested and samples were collected in tubes with RNAlater (per manufacturer's instructions) and flash frozen at −80° C. until DNA and RNA analysis (biodistribution of each vector in the pool) were performed by NGS (see FIGS. 36A-36H).

### 6.19 Capsid Amino Acid Sequences

Table 17 provides the amino acid sequences of certain engineered capsid proteins described and/or used in studies described herein. Heterologous peptides and amino acid substitutions are indicated in gray shading.

TABLE 17
Capsid Amino Acid Sequences

| Capsid | Insert or | |
|---|---|---|
| Name | Substitution | Amino Acid Sequence |
| PHP.S | QAVRTSL | 1 MAADGYLPDW LEDNLSEGIR EWWALKPGAP QPKANQQHQD NARGLVLPGY KYLGPGNGLD |
| (Califor- | (SEQ ID | 61 KGEPVNAADA AALEHDKAYD QQLKAGDNPY LKYNHADAEF QERLKEDTSF GGNLGRAVFQ |
| nia | NO: 16) | 121 AKKRLLEPLG LVEEAAKTAP GKKRPVEQSP QEPDSSAGIG KSGAQPAKKR LNFGQTGDTE |
| Institute | (588_589) | 181 SVPDPQPIGE PPAAPSGVGS LTMASGGGAP VADNNEGADG VGSSSGNWHC DSQWLGDRVI |
| of | | 241 TTSTRTWALP TYNNHLYKQI SNSTSGGSSN DNAYFGYSTP WGYFDFNRFH CHFSPRDWQR |
| Technol- | | 301 LINNNWGFRP KRLNFKLFNI QVKEVTDNNG VKTIANNLTS TVQVFTDSDY QLPYVLGSAH |
| ogy- | | 361 EGCLPPFPAD VFMIPQYGYL TLNDGSQAVG RSSFYCLEYF PSQMLRTGNN FQFSYEFENV |
| Chan et | | 421 PFHSSYAHSQ SLDRLMNPLI DQYLYYLSRT INGSGQNQQT LKFSVAGPSN MAVQGRNYIP |
| al 2017) | | 481 GPSYRQQRVS TTVTQNNNSE FAWPGASSWA LNGRNSLMNP GPAMASHKEG EDRFFPLSGS |
| | | 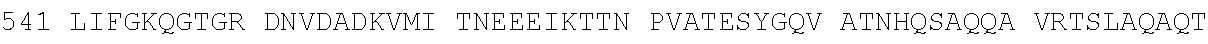 |
| | | 601 GWVQNQGILP GMVWQDRDVY LQGPIWAKIP HTDGNFHPSP LMGGFGMKHP PPQILIKNTP |
| | | 661 VPADPPTAFN KDKLNSFITQ YSTGQVSVEI EWELQKENSK RWNPEIQYTS NYYKSNNVEF |
| | | 721 AVNTEGVYSE PRPIGTRYLT RNL (SEQ ID NO: 22) |
| PHP.S | QAVRTSH | 1 MAADGYLPDW LEDNLSEGIR EWWALKPGAP QPKANQQHQD NARGLVLPGY KYLGPGNGLD |
| H | (SEQ ID | 61 KGEPVNAADA AALEHDKAYD QQLKAGDNPY LKYNHADAEF QERLKEDTSF GGNLGRAVFQ |
| | NO: 17) | 121 AKKRLLEPLG LVEEAAKTAP GKKRPVEQSP QEPDSSAGIG KSGAQPAKKR LNFGQTGDTE |
| | (588_589) | 181 SVPDPQPIGE PPAAPSGVGS LTMASGGGAP VADNNEGADG VGSSSGNWHC DSQWLGDRVI |
| | | 241 TTSTRTWALP TYNNHLYKQI SNSTSGGSSN DNAYFGYSTP WGYFDFNRFH CHFSPRDWQR |
| | | 301 LINNNWGFRP KRLNFKLFNI QVKEVTDNNG VKTIANNLTS TVQVFTDSDY QLPYVLGSAH |
| | | 361 EGCLPPFPAD VFMIPQYGYL TLNDGSQAVG RSSFYCLEYF PSQMLRTGNN FQFSYEFENV |
| | | 421 PFHSSYAHSQ SLDRLMNPLI DQYLYYLSRT INGSGQNQQT LKFSVAGPSN MAVQGRNYIP |
| | | 481 GPSYRQQRVS TTVTQNNNSE FAWPGASSWA LNGRNSLMNP GPAMASHKEG EDRFFPLSGS |
| | | 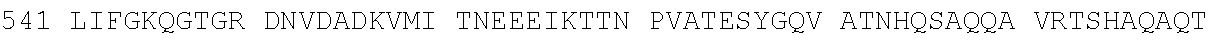 |
| | | 601 GWVQNQGILP GMVWQDRDVY LQGPIWAKIP HTDGNFHPSP LMGGFGMKHP PPQILIKNTP |
| | | 661 VPADPPTAFN KDKLNSFITQ YSTGQVSVEI EWELQKENSK RWNPEIQYTS NYYKSNNVEF |
| | | 721 AVNTEGVYSE PRPIGTRYLT RNL (SEQ ID NO: 23) |
| PHP.B | TLAVPFK | 1 MAADGYLPDW LEDNLSEGIR EWWALKPGAP QPKANQQHQD NARGLVLPGY KYLGPGNGLD |
| (Califor- | (SEQ ID | 61 KGEPVNAADA AALEHDKAYD QQLKAGDNPY LKYNHADAEF QERLKEDTSF GGNLGRAVFQ |
| nia | NO: 20) | 121 AKKRLLEPLG LVEEAAKTAP GKKRPVEQSP QEPDSSAGIG KSGAQPAKKR LNFGQTGDTE |
| Institute | (588_589) | 181 SVPDPQPIGE PPAAPSGVGS LTMASGGGAP VADNNEGADG VGSSSGNWHC DSQWLGDRVI |
| of | | 241 TTSTRTWALP TYNNHLYKQI SNSTSGGSSN DNAYFGYSTP WGYFDFNRFH CHFSPRDWQR |
| Technol- | | 301 LINNNWGFRP KRLNFKLFNI QVKEVTDNNG VKTIANNLTS TVQVFTDSDY QLPYVLGSAH |
| ogy | | 361 EGCLPPFPAD VFMIPQYGYL TLNDGSQAVG RSSFYCLEYF PSQMLRTGNN FQFSYEFENV |
| GenBank | | 421 PFHSSYAHSQ SLDRLMNPLI DQYLYYLSRT INGSGQNQQT LKFSVAGPSN MAVQGRNYIP |
| entry: | | 481 GPSYRQQRVS TTVTQNNNSE FAWPGASSWA LNGRNSLMNP GPAMASHKEG EDRFFPLSGS |
| ALU851 | | 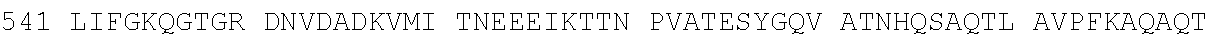 |
| 56.1- | | 601 GWVQNQGILP GMVWQDRDVY LQGPIWAKIP HTDGNFHPSP LMGGFGMKHP PPQILIKNTP |
| Deverman | | 661 VPADPPTAFN KDKLNSFITQ YSTGQVSVEI EWELQKENSK RWNPEIQYTS NYYKSNNVEF |
| et al | | 721 AVNTEGVYSE PRPIGTRYLT RNL (SEQ ID NO: 24) |
| 2016) | | |
| PHP.e | TLAVPFK | 1 MAADGYLPDW LEDNLSEGIR EWWALKPGAP QPKANQQHQD NARGLVLPGY KYLGPGNGLD |
| (Califor- | (SEQ ID | 61 KGEPVNAADA AALEHDKAYD QQLKAGDNPY LKYNHADAEF QERLKEDTSF GGNLGRAVFQ |
| nia | NO: 27) | 121 AKKRLLEPLG LVEEAAKTAP GKKRPVEQSP QEPDSSAGIG KSGAQPAKKR LNFGQTGDTE |
| Institute | (588_589) | 181 SVPDPQPIGE PPAAPSGVGS LTMASGGGAP VADNNEGADG VGSSSGNWHC DSQWLGDRVI |
| of | | 241 TTSTRTWALP TYNNHLYKQI SNSTSGGSSN DNAYFGYSTP WGYFDFNRFH CHFSPRDWQR |
| Technol- | | 301 LINNNWGFRP KRLNFKLFNI QVKEVTDNNG VKTIANNLTS TVQVFTDSDY QLPYVLGSAH |
| ogy- | | 361 EGCLPPFPAD VFMIPQYGYL TLNDGSQAVG RSSFYCLEYF PSQMLRTGNN FQFSYEFENV |
| Chan et | | 421 PFHSSYAHSQ SLDRLMNPLI DQYLYYLSRT INGSGQNQQT LKFSVAGPSN MAVQGRNYIP |
| al 2017) | | 481 GPSYRQQRVS TTVTQNNNSE FAWPGASSWA LNGRNSLMNP GPAMASHKEG EDRFFPLSGS |
| | | 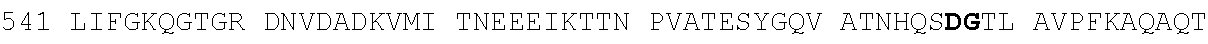 |
| | | 601 GWVQNQGILP GMVWQDRDVY LQGPIWAKIP HTDGNFHPSP LMGGFGMKHP PPQILIKNTP |
| | | 661 VPADPPTAFN KDKLNSFITQ YSTGQVSVEI EWELQKENSK RWNPEIQYTS NYYKSNNVEF |
| | | 721 AVNTEGVYSE PRPIGTRYLT RNL (SEQ ID NO: 25) |
| AAV8. | A269S | MAADGYLPDW LEDNLSEGIR EWWALKPGAP KPKANQQKQD DGRGLVLPGY KYLGPFNGLD 60 |
| BBB | | KGEPVNAADA AALEHDKAYD QQLQAGDNPY LRYNHADAEF QERLQEDTSF GGNLGRAVFQ 120 |
| | | AKKRVLEPLG LVEEGAKTAP GKKRPVEPSP QRSPDSSTGI GKKGQQPARK RLNFGQTGDS 180 |
| | | ESVPDPQPLG EPPAAPSGVG PNTMAAGGGA PMADNNEGAD GVGSSSGNWH CDSTWLGDRV 240 |
| | | 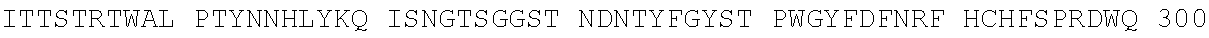 |
| | | RLINNNWGFR PKRLSFKLFN IQVKEVTQNE GTKTIANNLT STIQVFTDSE YQLPYVLGSA 360 |
| | | HQGCLPPFPA DVFMIPQYGY LTLNNGSQAV GRSSFYCLEY FPSQMLRTGN NFQFTYTFED 420 |
| | | VPFHSSYAHS QSLDRLMNPL IDQYLYYLSR TQTTGGTANT QTLGFSQGGP NTMANQAKNW 480 |
| | | LPGPCYRQQR VSTTTGQNNN SNFAWTAGTK YHINGRNSLA NPGIAMATHK DDEERFFPSN 540 |
| | | GILIFGKQNA ARDNADYSDV MLTSEEEIKT TNPVATEEYG IVADNLQQQN TAPQIGTVNS 600 |
| | | QGALPGMVWQ NRDVYLQGPI WAKIPHTDGN FHPSPLMGGF GLKHPPPQIL IKNTPVPADP 660 |
| | | PTTFNQSKLN SFITQYSTGQ VSVEIEWELQ KENSKRWNPE IQYTSNYYKS TSVDFAVNTE 720 |
| | | GVYSEPRPIG TRYLTRNL (SEQ ID NO: 26) |
| AAV8. | A269S, | MAADGYLPDW LEDNLSEGIR EWWALKPGAP KPKANQQKQD DGRGLVLPGY KYLGPFNGLD 60 |
| BBB. | 498_NNN/ | KGEPVNAADA AALEHDKAYD QQLQAGDNPY LRYNHADAEF QERLQEDTSF GGNLGRAVFQ 120 |
| LD | AAA_500 | AKKRVLEPLG LVEEGAKTAP GKKRPVEPSP QRSPDSSTGI GKKGQQPARK RLNFGQTGDS 180 |
| | | ESVPDPQPLG EPPAAPSGVG PNTMAAGGGA PMADNNEGAD GVGSSSGNWH CDSTWLGDRV 240 |
| | | 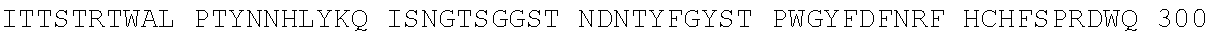 |
| | | RLINNNWGFR PKRLSFKLFN IQVKEVTQNE GTKTIANNLT STIQVFTDSE YQLPYVLGSA 360 |
| | | HQGCLPPFPA DVFMIPQYGY LTLNNGSQAV GRSSFYCLEY FPSQMLRTGN NFQFTYTFED 420 |
| | | VPFHSSYAHS QSLDRLMNPL IDQYLYYLSR TQTTGGTANT QTLGFSQGGP NTMANQAKNW 480 |
| | | 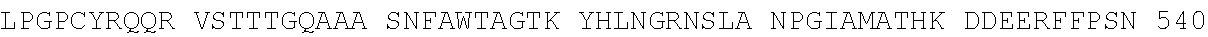 |
| | | GILIFGKQNA ARDNADYSDV MLTSEEEIKT TNPVATEEYG IVADNLQQQN TAPQIGTVNS 600 |
| | | QGALPGMVWQ NRDVYLQGPI WAKIPHTDGN FHPSPLMGGF GLKHPPPQIL IKNTPVPADP 660 |
| | | PTTFNQSKLN SFITQYSTGQ VSVEIEWELQ KENSKRWNPE IQYTSNYYKS TSVDFAVNTE 720 |
| | | GVYSEPRPIG TRYLTRNL (SEQ ID NO: 27) |
| AAV9. | S263G/ | MAADGYLPDW LEDNLSEGIR EWWALKPGAP QPKANQQHQD NARGLVLPGY KYLGPGNGLD 60 |
| BBB | S269T/ | KGEPVNAADA AALEHDKAYD QQLKAGDNPY LKYNHADAEF QERLKEDTSF GGNLGRAVFQ 120 |
| | A273T | AKKRLLEPLG LVEEAAKTAP GKKRPVEQSP QEPDSSAGIG KSGAQPAKKR LNFGQTGDTE 180 |
| | | SVPDPQPIGE PPAAPSGVGS LTMASGGGAP VADNNEGADG VGSSSGNWHC DSQWLGDRVI 240 |
| | | 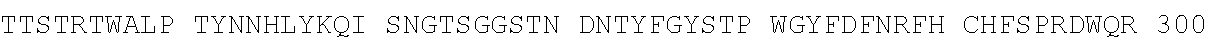 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | GPSYRQQRVS TTVTQNNNSE FAWPGASSWA LNGRNSLMNP GPAMASHKEG EDRFFPLSGS 540 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 28) |
| AAV9. | S263G/ | MAADGYLPDW LEDNLSEGIR EWWALKPGAP QPKANQQHQD NARGLVLPGY KYLGPGNGLD 60 |
| BBB. | S269T/ | KGEPVNAADA AALEHDKAYD QQLKAGDNPY LKYNHADAEF QERLKEDTSF GGNLGRAVFQ 120 |
| LD | A273T, | AKKRLLEPLG LVEEAAKTAP GKKRPVEQSP QEPDSSAGIG KSGAQPAKKR LNFGQTGDTE 180 |
| | 496_NNN/ | SVPDPQPIGE PPAAPSGVGS LTMASGGGAP VADNNEGADG VGSSSGNWHC DSQWLGDRVI 240 |
| | AAA_498 | 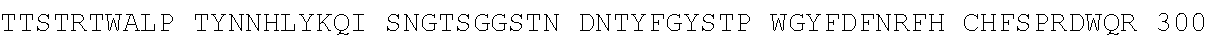 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 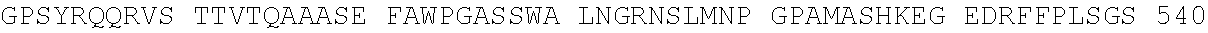 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 29) |
| AAVrh. | 498_NNN/ | MAADGYLPDW LEDNLSEGIR EWWDLKPGAP KPKANQQKQD DGRGLVLPGY 50 |
| 10.LD | AAA_500 | KYLGPFNGLD KGEPVNAADA AALEHDKAYD QQLKAGDNPY LRYNHADAEF 100 |
| | | QERLQEDTSF GGNLGRAVFQ AKKRVLEPLG LVEEGAKTAP GKKRPVEPSP 150 |
| | | QRSPDSSTGI GKKGQQPAKK RLNFGQTGDS ESVPDPQPIG EPPAGPSGLG 200 |
| | | SGTMAAGGGA PMADNNEGAD GVGSSSGNWH CDSTWLGDRV ITTSTRTWAL 250 |
| | | PTYNNHLYKQ ISNGTSGGST NDNTYFGYST PWGYFDFNRF HCHFSPRDWQ 300 |
| | | RLINNNWGFR PKRLNFKLFN IQVKEVTQNE GTKTIANNLT STIQVFTDSE 350 |
| | | YQLPYVLGSA HQGCLPPFPA DVFMIPQYGY LTLNNGSQAV GRSSFYCLEY 400 |
| | | FPSQMLRTGN NFEFSYQFED VPFHSSYAHS QSLDRLMNPL IDQYLYYLSR 450 |
| | | 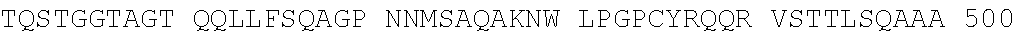 |
| | | SNFAWTGATK YHLNGRDSLV NPGVAMATHK DDEERFFPSS GVLMFGKQGA 550 |
| | | GKDNVDYSSV MLTSEEEIKT TNPVATEQYG VVADNLQQQN AAPIVGAVNS 600 |
| | | QGALPGMVWQ NRDVYLQGPI WAKIPHTDGN FHPSPLMGGF GLKHPPPQIL 650 |
| | | IKNTPVPADP PTTFSQAKLA SFITQYSTGQ VSVEIEWELQ KENSKRWNPE 700 |
| | | IQYTSNYYKS TNVDFAVNTD GTYSEPRPIG TRYLTRNL (SEQ ID NO: 30) |
| AAV9. | 498_NNN/ | MAADGYLPDW LEDNLSEGIR EWWALKPGAP QPKANQQHQD NARGLVLPGY KYLGPGNGLD 60 |
| 496NN | AAA_500 | KGEPVNAADA AALEHDKAYD QQLKAGDNPY LKYNHADAEF QERLKEDTSF GGNLGRAVFQ 120 |
| N/AA | | AKKRLLEPLG LVEEAAKTAP GKKRPVEQSP QEPDSSAGIG KSGAQPAKKR LNFGQTGDTE 180 |
| A498 | | SVPDPQPIGE PPAAPSGVGS LTMASGGGAP VADNNEGADG VGSSSGNWHC DSQWLGDRVI 240 |
| | | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 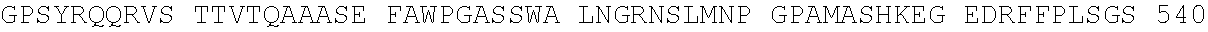 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 31) |
| AAV9. | 496NNN/ | MAADGYLPDW LEDNLSEGIR EWWALKPGAP QPKANQQHQD NARGLVLPGY KYLGPGNGLD 60 |
| 496NN | AAA498, | KGEPVNAADA AALEHDKAYD QQLKAGDNPY LKYNHADAEF QERLKEDTSF GGNLGRAVFQ 120 |
| N/AA | W503R | AKKRLLEPLG LVEEAAKTAP GKKRPVEQSP QEPDSSAGIG KSGAQPAKKR LNFGQTGDTE 180 |
| A498. | | SVPDPQPIGE PPAAPSGVGS LTMASGGGAP VADNNEGADG VGSSSGNWHC DSQWLGDRVI 240 |
| W503 | | TTSTRTWALP TYNNHLYKQI SNSTSGGSSN DNAYFGYSTP WGYFDFNRFH CHFSPRDWQR 300 |
| R | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 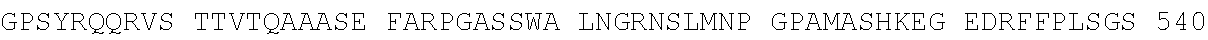 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 32) |
| AAV9 | W503R | MAADGYLPDW LEDNLSEGIR EWWALKPGAP QPKANQQHQD NARGLVLPGY KYLGPGNGLD 60 |
| W503 | | KGEPVNAADA AALEHDKAYD QQLKAGDNPY LKYNHADAEF QERLKEDTSF GGNLGRAVFQ 120 |
| R | | AKKRLLEPLG LVEEAAKTAP GKKRPVEQSP QEPDSSAGIG KSGAQPAKKR LNFGQTGDTE 180 |
| | | SVPDPQPIGE PPAAPSGVGS LTMASGGGAP VADNNEGADG VGSSSGNWHC DSQWLGDRVI 240 |
| | | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 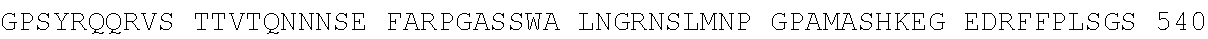 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 33) |
| AAV9 | Q474A | MAADGYLPDW LEDNLSEGIR EWWALKPGAP QPKANQQHQD NARGLVLPGY KYLGPGNGLD 60 |
| Q474A | | KGEPVNAADA AALEHDKAYD QQLKAGDNPY LKYNHADAEF QERLKEDTSF GGNLGRAVFQ 120 |
| | | AKKRLLEPLG LVEEAAKTAP GKKRPVEQSP QEPDSSAGIG KSGAQPAKKR LNFGQTGDTE 180 |
| | | SVPDPQPIGE PPAAPSGVGS LTMASGGGAP VADNNEGADG VGSSSGNWHC DSQWLGDRVI 240 |
| | | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 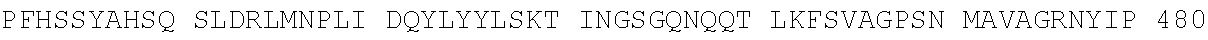 |
| | | GPSYRQQRVS TTVTQNNNSE FAWPGASSWA LNGRNSLMNP GPAMASHKEG EDRFFPLSGS 540 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 34) |
| AAV9 | Bone1, | MAADGYLPDW LEDNLSEGIR EWWALKPGAP QPKANQQHQD NARGLVLPGY KYLGPGNGLD 60 |
| S454- | DDDDDDD | KGEPVNAADA AALEHDKAYD QQLKAGDNPY LKYNHADAEF QERLKEDTSF GGNLGRAVFQ 120 |
| D8 | D (SEQ ID | AKKRLLEPLG LVEEAAKTAP GKKRPVEQSP QEPDSSAGIG KSGAQPAKKR LNFGQTGDTE 180 |
| | NO: 9) | SVPDPQPIGE PPAAPSGVGS LTMASGGGAP VADNNEGADG VGSSSGNWHC DSQWLGDRVI 240 |
| | (454_455) | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 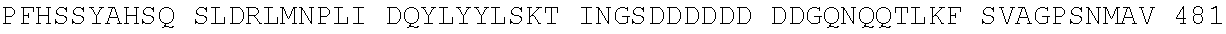 |
| | | QGRNYIPGPS YRQQRVSTTV TQNNNSEFAW PGASSWALNG RNSLMNPGPA MASHKEGEDR 541 |
| | | FFPLSGSLIF GKQGTGRDNV DADKVMITNE EEIKTTNPVA TESYGQVATN HQSAQAQAQT 601 |
| | | GWVQNQGILP GMVWQDRDVY LQGPIWAKIP HTDGNFHPSP LMGGFGMKHP PPQILIKNTP 661 |
| | | VPADPPTAFN KDKLNSFITQ YSTGQVSVEI EWELQKENSK RWNPEIQYTS NYYKSNNVEF 721 |
| | | AVNTEGVYSE PRPIGTRYLT RNL (SEQ ID NO:35) |
| AAV9 | Brain1, | MAADGYLPDW LEDNLSEGIR EWWALKPGAP QPKANQQHQD NARGLVLPGY KYLGPGNGLD 60 |
| S454- | LSSRLDA | KGEPVNAADA AALEHDKAYD QQLKAGDNPY LKYNHADAEF QERLKEDTSF GGNLGRAVFQ 120 |
| Brain 1 | (SEQ ID | AKKRLLEPLG LVEEAAKTAP GKKRPVEQSP QEPDSSAGIG KSGAQPAKKR LNFGQTGDTE 180 |
| | NO: 10) | SVPDPQPIGE PPAAPSGVGS LTMASGGGAP VADNNEGADG VGSSSGNWHC DSQWLGDRVI 240 |
| | (454_455) | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 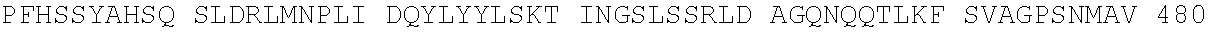 |
| | | QGRNYIPGPS YRQQRVSTTV TQNNNSEFAW PGASSWALNG RNSLMNPGPA MASHKEGEDR 540 |
| | | FFPLSGSLIF GKQGTGRDNV DADKVMITNE EEIKTTNPVA TESYGQVATN HQSAQAQAQT 600 |
| | | GWVQNQGILP GMVWQDRDVY LQGPIWAKIP HTDGNFHPSP LMGGFGMKHP PPQILIKNTP 660 |
| | | VPADPPTAFN KDKLNSFITQ YSTGQVSVEI EWELQKENSK RWNPEIQYTS NYYKSNNVEF 720 |
| | | AVNTEGVYSE PRPIGTRYLT RNL (SEQ ID NO: 36) |
| AAV9 | Brain2/ | MAADGYLPDW LEDNLSEGIR EWWALKPGAP QPKANQQHQD NARGLVLPGY KYLGPGNGLD 60 |
| S454- | Brain 1C, | KGEPVNAADA AALEHDKAYD QQLKAGDNPY LKYNHADAEF QERLKEDTSF GGNLGRAVFQ 120 |
| Brain2 | CLSSRLDA | AKKRLLEPLG LVEEAAKTAP GKKRPVEQSP QEPDSSAGIG KSGAQPAKKR LNFGQTGDTE 180 |
| | C (SEQ ID | SVPDPQPIGE PPAAPSGVGS LTMASGGGAP VADNNEGADG VGSSSGNWHC DSQWLGDRVI 240 |
| | NO: 11) | TTSTRTWALP TYNNHLYKQI SNSTSGGSSN DNAYFGYSTP WGYFDFNRFH CHFSPRDWQR 300 |
| | (454_455) | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 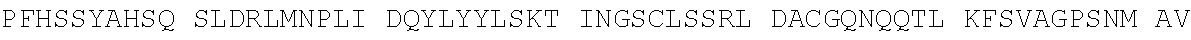 |
| | | 482 |
| | | QGRNYIPGPS YRQQRVSTTV TQNNNSEFAW PGASSWALNG RNSLMNPGPA MASHKEGEDR 542 |
| | | FFPLSGSLIF GKQGTGRDNV DADKVMITNE EEIKTTNPVA TESYGQVATN HQSAQAQAQT 602 |
| | | GWVQNQGILP GMVWQDRDVY LQGPIWAKIP HTDGNFHPSP LMGGFGMKHP PPQILIKNTP 662 |
| | | VPADPPTAFN KDKLNSFITQ YSTGQVSVEI EWELQKENSK RWNPEIQYTS NYYKSNNVEF 722 |
| | | AVNTEGVYSE PRPIGTRYLT RNL (SEQ ID NO: 37) |
| AAV9 | Kidney 1, | MAADGYLPDW LEDNLSEGIR EWWALKPGAP QPKANQQHQD NARGLVLPGY KYLGPGNGLD 60 |
| S454- | LPVAS | KGEPVNAADA AALEHDKAYD QQLKAGDNPY LKYNHADAEF QERLKEDTSF GGNLGRAVFQ 120 |
| Kidney | (SEQ ID | AKKRLLEPLG LVEEAAKTAP GKKRPVEQSP QEPDSSAGIG KSGAQPAKKR LNFGQTGDTE 180 |
| 1 | NO: 13) | SVPDPQPIGE PPAAPSGVGS LTMASGGGAP VADNNEGADG VGSSSGNWHC DSQWLGDRVI 240 |
| | (454_455) | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 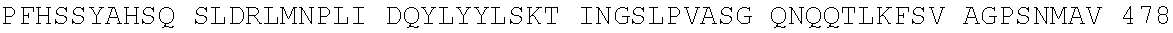 |
| | | QGRNYIPGPS YRQQRVSTTV TQNNNSEFAW PGASSWALNG RNSLMNPGPA MASHKEGEDR 538 |
| | | FFPLSGSLIF GKQGTGRDNV DADKVMITNE EEIKTTNPVA TESYGQVATN HQSAQAQAQT 598 |
| | | GWVQNQGILP GMVWQDRDVY LQGPIWAKIP HTDGNFHPSP LMGGFGMKHP PPQILIKNTP 658 |
| | | VPADPPTAFN KDKLNSFITQ YSTGQVSVEI EWELQKENSK RWNPEIQYTS NYYKSNNVEF 718 |
| | | AVNTEGVYSE PRPIGTRYLT RNL (SEQ ID NO: 38) |
| AAV9 | Kidney 2/ | MAADGYLPDW LEDNLSEGIR EWWALKPGAP QPKANQQHQD NARGLVLPGY KYLGPGNGLD 60 |
| S454- | Kidney 1C, | KGEPVNAADA AALEHDKAYD QQLKAGDNPY LKYNHADAEF QERLKEDTSF GGNLGRAVFQ 120 |
| Kidney | CLPVASC | AKKRLLEPLG LVEEAAKTAP GKKRPVEQSP QEPDSSAGIG KSGAQPAKKR LNFGQTGDTE 180 |
| 2 | (SEQ ID | SVPDPQPIGE PPAAPSGVGS LTMASGGGAP VADNNEGADG VGSSSGNWHC DSQWLGDRVI 240 |
| | NO: 12) | TTSTRTWALP TYNNHLYKQI SNSTSGGSSN DNAYFGYSTP WGYFDFNRFH CHFSPRDWQR 300 |
| | (454_455) | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 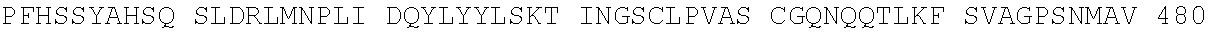 |
| | | QGRNYIPGPS YRQQRVSTTV TQNNNSEFAW PGASSWALNG RNSLMNPGPA MASHKEGEDR 540 |
| | | FFPLSGSLIF GKQGTGRDNV DADKVMITNE EEIKTTNPVA TESYGQVATN HQSAQAQAQT 600 |
| | | GWVQNQGILP GMVWQDRDVY LQGPIWAKIP HTDGNFHPSP LMGGFGMKHP PPQILIKNTP 660 |
| | | VPADPPTAFN KDKLNSFITQ YSTGQVSVEI EWELQKENSK RWNPEIQYTS NYYKSNNVEF 720 |
| | | AVNTEGVYSE PRPIGTRYLT RNL (SEQ ID NO: 39) |
| AAV9 | Muscle1, | MAADGYLPDW LEDNLSEGIR EWWALKPGAP QPKANQQHQD NARGLVLPGY KYLGPGNGLD 60 |
| S454- | ASSLNIA | KGEPVNAADA AALEHDKAYD QQLKAGDNPY LKYNHADAEF QERLKEDTSF GGNLGRAVFQ 120 |
| Muscle | (SEQ ID | AKKRLLEPLG LVEEAAKTAP GKKRPVEQSP QEPDSSAGIG KSGAQPAKKR LNFGQTGDTE 180 |
| 1 | NO: 14) | SVPDPQPIGE PPAAPSGVGS LTMASGGGAP VADNNEGADG VGSSSGNWHC DSQWLGDRVI 240 |
| | (454_455) | TTSTRTWALP TYNNHLYKQI SNSTSGGSSN DNAYFGYSTP WGYFDFNRFH CHFSPRDWQR 300 |
| | | EGCLPPFPAD VFMIPQYGYL TLNDGSQAVG RSSFYCLEYF PSQMLRTGNN FQFSYEFENV 420 |
| | | 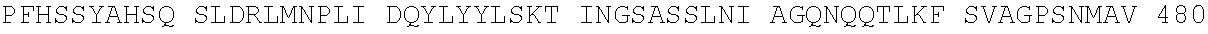 |
| | | QGRNYIPGPS YRQQRVSTTV TQNNNSEFAW PGASSWALNG RNSLMNPGPA MASHKEGEDR 540 |
| | | FFPLSGSLIF GKQGTGRDNV DADKVMITNE EEIKTTNPVA TESYGQVATN HQSAQAQAQT 600 |
| | | GWVQNQGILP GMVWQDRDVY LQGPIWAKIP HTDGNFHPSP LMGGFGMKHP PPQILIKNTP 660 |
| | | VPADPPTAFN KDKLNSFITQ YSTGQVSVEI EWELQKENSK RWNPEIQYTS NYYKSNNVEF 720 |
| | | AVNTEGVYSE PRPIGTRYLT RNL (SEQ ID NO: 40) |
| AAV9 | Tfr1, | MAADGYLPDW LEDNLSEGIR EWWALKPGAP QPKANQQHQD NARGLVLPGY KYLGPGNGLD 60 |
| S454- | HAIYPR | KGEPVNAADA AALEHDKAYD QQLKAGDNPY LKYNHADAEF QERLKEDTSF GGNLGRAVFQ 120 |
| Trfr1 | (SEQ ID | AKKRLLEPLG LVEEAAKTAP GKKRPVEQSP QEPDSSAGIG KSGAQPAKKR LNFGQTGDTE 180 |
| | NO: 59) | SVPDPQPIGE PPAAPSGVGS LTMASGGGAP VADNNEGADG VGSSSGNWHC DSQWLGDRVI 240 |
| | (454_455) | LINNNWGFRP KRLNFKLFNI QVKEVTDNNG VKTIANNLTS TVQVFTDSDY QLPYVLGSAH 360 |
| | | TTSTRTWALP TYNNHLYKQI SNSTSGGSSN DNAYFGYSTP WGYFDFNRFH CHFSPRDWQR 300 |
| | | EGCLPPFPAD VFMIPQYGYL TLNDGSQAVG RSSFYCLEYF PSQMLRTGNN FQFSYEFENV 420 |
| | | 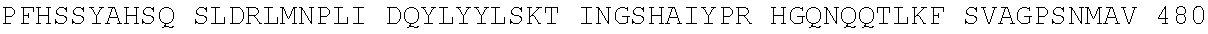 |
| | | QGRNYIPGPS YRQQRVSTTV TQNNNSEFAW PGASSWALNG RNSLMNPGPA MASHKEGEDR 540 |
| | | FFPLSGSLIF GKQGTGRDNV DADKVMITNE EEIKTTNPVA TESYGQVATN HQSAQAQAQT 600 |
| | | GWVQNQGILP GMVWQDRDVY LQGPIWAKIP HTDGNFHPSP LMGGFGMKHP PPQILIKNTP 660 |
| | | VPADPPTAFN KDKLNSFITQ YSTGQVSVEI EWELQKENSK RWNPEIQYTS NYYKSNNVEF 720 |
| | | AVNTEGVYSE PRPIGTRYLT RNL (SEQ ID NO: 41) |
| AAV9 | Tfr3, | MAADGYLPDW LEDNLSEGIR EWWALKPGAP QPKANQQHQD NARGLVLPGY KYLGPGNGLD 60 |
| S454- | RTIGPSV | KGEPVNAADA AALEHDKAYD QQLKAGDNPY LKYNHADAEF QERLKEDTSF GGNLGRAVFQ 120 |
| Tfr3 | (SEQ ID | AKKRLLEPLG LVEEAAKTAP GKKRPVEQSP QEPDSSAGIG KSGAQPAKKR LNFGQTGDTE 180 |
| | NO: 19) | SVPDPQPIGE PPAAPSGVGS LTMASGGGAP VADNNEGADG VGSSSGNWHC DSQWLGDRVI 240 |
| | (454_455) | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 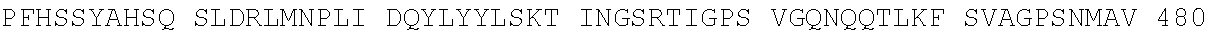 |
| | | QGRNYIPGPS YRQQRVSTTV TQNNNSEFAW PGASSWALNG RNSLMNPGPA MASHKEGEDR 540 |
| | | FFPLSGSLIF GKQGTGRDNV DADKVMITNE EEIKTTNPVA TESYGQVATN HQSAQAQAQT 600 |
| | | GWVQNQGILP GMVWQDRDVY LQGPIWAKIP HTDGNFHPSP LMGGFGMKHP PPQILIKNTP 660 |
| | | VPADPPTAFN KDKLNSFITQ YSTGQVSVEI EWELQKENSK RWNPEIQYTS NYYKSNNVEF 720 |
| | | AVNTEGVYSE PRPIGTRYLT RNL (SEQ ID NO: 42) |
| AAV9 | Tfr4, | MAADGYLPDW LEDNLSEGIR EWWALKPGAP QPKANQQHQD NARGLVLPGY KYLGPGNGLD 60 |
| S454- | CRTIGPSV | KGEPVNAADA AALEHDKAYD QQLKAGDNPY LKYNHADAEF QERLKEDTSF GGNLGRAVFQ 120 |
| Tfr4 | C (SEQ ID | AKKRLLEPLG LVEEAAKTAP GKKRPVEQSP QEPDSSAGIG KSGAQPAKKR LNFGQTGDTE 180 |
| (AAV9 | NO: 20) | SVPDPQPIGE PPAAPSGVGS LTMASGGGAP VADNNEGADG VGSSSGNWHC DSQWLGDRVI 240 |
| S454- | (454_455) | TTSTRTWALP TYNNHLYKQI SNSTSGGSSN DNAYFGYSTP WGYFDFNRFH CHFSPRDWQR 300 |
| TfR3C) | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | 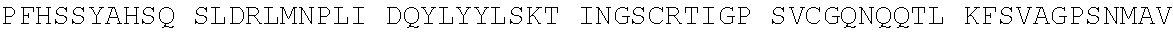 |
| | | 482 |
| | | QGRNYIPGPS YRQQRVSTTV TQNNNSEFAW PGASSWALNG RNSLMNPGPA MASHKEGEDR 542 |
| | | FFPLSGSLIF GKQGTGRDNV DADKVMITNE EEIKTTNPVA TESYGQVATN HQSAQAQAQT 602 |
| | | GWVQNQGILP GMVWQDRDVY LQGPIWAKIP HTDGNFHPSP LMGGFGMKHP PPQILIKNTP 662 |
| | | VPADPPTAFN KDKLNSFITQ YSTGQVSVEI EWELQKENSK RWNPEIQYTS NYYKSNNVEF 722 |
| | | AVNTEGVYSE PRPIGTRYLT RNL (SEQ ID NO: 43) |
| AAV9. | SITLVKST | MAADGYLPDW LEDNLSEGIR EWWALKPGAP QPKANQQHQD NARGLVLPGY KYLGPGNGLD 60 |
| 588Ad | QTV (SEQ | KGEPVNAADA AALEHDKAYD QQLKAGDNPY LKYNHADAEF QERLKEDTSF GGNLGRAVFQ 120 |
| (9 588 | ID NO: | AKKRLLEPLG LVEEAAKTAP GKKRPVEQSP QEPDSSAGIG KSGAQPAKKR LNFGQTGDTE 180 |
| Ad) | 21), | SVPDPQPIGE PPAAPSGVGS LTMASGGGAP VADNNEGADG VGSSSGNWHC DSQWLGDRVI 240 |
| | DLC-AS1 | TTSTRTWALP TYNNHLYKQI SNSTSGGSSN DNAYFGYSTP WGYFDFNRFH CHFSPRDWQR 300 |
| | (588_589) | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 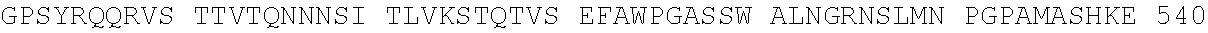 |
| | | GEDRFFPLSG SLIFGKQGTG RDNVDADKVM ITNEEEIKTT NPVATESYGQ VATNHQSAQA 600 |
| | | QAQTGWVQNQ GILPGMVWQD RDVYLQGPIW AKIPHTDGNF HPSPLMGGFG MKHPPPQILI 660 |
| | | KNTPVPADPP TAFNKDKLNS FITQYSTGQV SVEIEWELQK ENSKRWNPEI QYTSNYYKSN 720 |
| | | NVEFAVNTEG VYSEPRPIGT RYLTRNL (SEQ ID NO: 44) |
| AAV9. | TILSRSTQ | MAADGYLPDW LEDNLSEGIR EWWALKPGAP QPKANQQHQD NARGLVLPGY KYLGPGNGLD 60 |
| 588Ad | TG (SEQ | AKKRLLEPLG LVEEAAKTAP GKKRPVEQSP QEPDSSAGIG KSGAQPAKKR LNFGQTGDTE 180 |
| Herp | ID NO: | SVPDPQPIGE PPAAPSGVGS LTMASGGGAP VADNNEGADG VGSSSGNWHC DSQWLGDRVI 240 |
| (9 588 | 22), DLC- | TTSTRTWALP TYNNHLYKQI SNSTSGGSSN DNAYFGYSTP WGYFDFNRFH CHFSPRDWQR 300 |
| Hep) | AS2, | LINNNWGFRP KRLNFKLFNI QVKEVTDNNG VKTIANNLTS TVQVFTDSDY QLPYVLGSAH 360 |
| | 588_589 | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 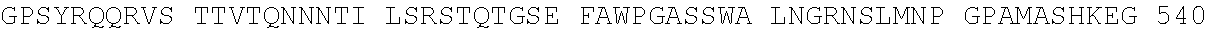 |
| | | EDRFFPLSGS LIFGKQGTGR DNVDADKVMI TNEEEIKTTN PVATESYGQV ATNHQSAQAQ 600 |
| | | AQTGWVQNQG ILPGMVWQDR DVYLQGPIWA KIPHTDGNFH PSPLMGGFGM KHPPPQILIK 660 |
| | | NTPVPADPPT AFNKDKLNSF ITQYSTGQVS VEIEWELQKE NSKRWNPEIQ YTSNYYKSNN 720 |
| | | VEFAVNTEG VYSEPRPIGT RYLTRNL (SEQ ID NO: 45) |
| AAVP | SITLVKST | 1 MAADGYLPDW LEDNLSEGIR EWWALKPGAP QPKANQQHQD NARGLVLPGY KYLGPGNGLD |
| HPeB. | QTV (SEQ | 61 KGEPVNAADA AALEHDKAYD QQLKAGDNPY LKYNHADAEF QERLKEDTSF GGNLGRAVFQ |
| VP2Ad | ID NO: | 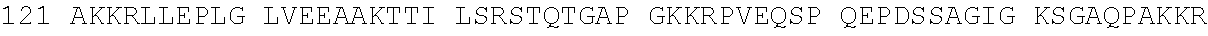 |
| 588 | 21), DLC- | LNFGQTGDTE |
| | AS1 | 191 SVPDPQPIGE PPAAPSGVGS LTMASGGGAP VADNNEGADG VGSSSGNWHC DSQWLGDRVI |
| | (138_139) | 251 TTSTRTWALP TYNNHLYKQI SNSTSGGSSN DNAYFGYSTP WGYFDFNRFH CHFSPRDWQR |
| | | 311 LINNNWGFRP KRLNFKLFNI QVKEVTDNNG VKTIANNLTS TVQVFTDSDY QLPYVLGSAH |
| | | 371 EGCLPPFPAD VFMIPQYGYL TLNDGSQAVG RSSFYCLEYF PSQMLRTGNN FQFSYEFENV |
| | | 431 PFHSSYAHSQ SLDRLMNPLI DQYLYYLSRT INGSGQNQQT LKFSVAGPSN MAVQGRNYIP |
| | | 491 GPSYRQQRVS TTVTQNNNSE FAWPGASSWA LNGRNSLMNP GPAMASHKEG EDRFFPLSGS |
| | | 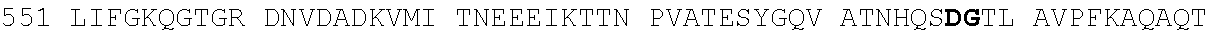 |
| | | 611 GWVQNQGILP GMVWQDRDVY LQGPIWAKIP HTDGNFHPSP LMGGFGMKHP PPQILIKNTP |
| | | 671 VPADPPTAFN KDKLNSFITQ YSTGQVSVEI EWELQKENSK RWNPEIQYTS NYYKSNNVEF |
| | | 731 AVNTEGVYSE PRPIGTRYLT RNL (SEQ ID NO: 46) |
| AAVP | TILSRSTQ | 1 MAADGYLPDW LEDNLSEGIR EWWALKPGAP QPKANQQHQD NARGLVLPGY KYLGPGNGLD |
| HPeB. | TG (SEQ | 61 KGEPVNAADA AALEHDKAYD QQLKAGDNPY LKYNHADAEF QERLKEDTSF GGNLGRAVFQ |
| VP2He | ID NO: | 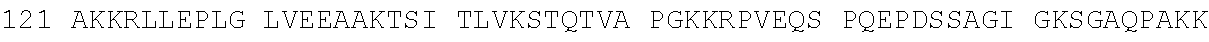 |
| rp | 22), DLC- | RLNFGQTGDT E |
| | AS2, | 192 SVPDPQPIGE PPAAPSGVGS LTMASGGGAP VADNNEGADG VGSSSGNWHC DSQWLGDRVI |
| | (138_139) | 252 TTSTRTWALP TYNNHLYKQI SNSTSGGSSN DNAYFGYSTP WGYFDFNRFH CHFSPRDWQR |
| | | 312 LINNNWGFRP KRLNFKLFNI QVKEVTDNNG VKTIANNLTS TVQVFTDSDY QLPYVLGSAH |
| | | 372 EGCLPPFPAD VFMIPQYGYL TLNDGSQAVG RSSFYCLEYF PSQMLRTGNN FQFSYEFENV |
| | | 432 PFHSSYAHSQ SLDRLMNPLI DQYLYYLSRT INGSGQNQQT LKFSVAGPSN MAVQGRNYIP |
| | | 492 GPSYRQQRVS TTVTQNNNSE FAWPGASSWA LNGRNSLMNP GPAMASHKEG EDRFFPLSGS |
| | | 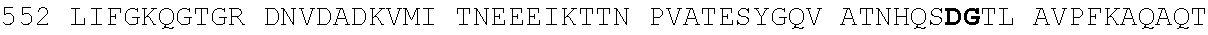 |
| | | 612 GWVQNQGILP GMVWQDRDVY LQGPIWAKIP HTDGNFHPSP LMGGFGMKHP PPQILIKNTP |
| | | 672 VPADPPTAFN KDKLNSFITQ YSTGQVSVEI EWELQKENSK RWNPEIQYTS NYYKSNNVEF |
| | | 732 AVNTEGVYSE PRPIGTRYLT RNL (SEQ ID NO: 47) |
| rh34 | — | MAADGYLPDWLEDNLSEGIREWWDLKPGAPKPKANQQKQDDGRGLVLPGYEYLGPFNGLDKGEPVNAAD |
| | | AAALEHDKAYDQQLKAGDNPYLRYNHADAEFQERLQEDTSFGGNLGRAVFQAKKRVLEPLGLVEEGAKT |
| | | APGKKRPLESPQEPDSSSGIGKKGKQPAKKRLNFEEDTGAGDGPPEGSDTSAMSSDIEMRAAPGGNAVD |
| | | AGQGSDGVGNASGDWHCDSTWSEGKVTTTSTRTWVLPTYNNHLYLRLGTTSNSNTYNGFSTPWGYFDFN |
| | | RFHCHFSPRDWQRLINNNWGLRPKAMRVKIFNIQVKEVTTSNGETTVANNLTSTVQIFADSSYELPYVM |
| | | DAGQEGSLPPFPNDVFMVPQYGYCGIVTGENQNQTDRNAFYCLEYFPSQMLRTGNNFETAYNFEKVPFH |
| | | SMYAHSQSLDGLMNPLLDQYLWHLQSTTSGETLNQGNAATTFGKIRSGDFAFYRKNWLPGPCVKQQRFS |
| | | KTASQNYKIPASGGNALLKYDTHYTLNNRWSNIAPGPPMATAGPSDGDFSNAQLIFPGPSVTGNTTTSA |
| | | NNLLFTSEEEIAATNPRDTDMFGQIADNNQNATTAPITGNVTAMGVLPGMVWQNRDIYYQGPIWAKIPH |
| | | ADGHFHPSPLIGGFGLKHPPPQIFIKNTPVPAYPATTFTAARVDSFITQYSTGQVAVQIEWEIEKERSK |
| | | RWNPEVQFTSNCGNQSSMLWAPDTTGKYTEPRVIGSRYLTNHL (SEQ ID NO: 82) |
| hu.31 | — | MAADGYLPDW LEDTLSEGIR QWWKLKPGPP PPKPAERHKD DSRGLVLPGY |
| | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGSQPAKKK LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY |
| | | QLPYVLGSAH EGCLPPFPAD VFMIPQYGYL TLNDGGQAVG RSSFYCLEYF |
| | | PSQMLRTGNN FQFSYEFENV PFHSSYAHSQ SLDRLMNPLI DQYLYYLSKT |
| | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSAQAQ AQTGWVQNQG |
| | | ILPGMVWQDR DVYLQGPIWA KIPHTDGNFH PSPLMGGFGM KHPPPQILIK |
| | | NTPVPADPPT AFNKDKLNSF ITQYSTGQVS VEIEWELQKE NSKRWNPEIQ |
| | | YTSNYYKSNN VEFAVSTEGV YSEPRPIGTR YLTRNL (SEQ ID NO:83) |
| rh.31 | — | KAYDQQLKAG DNPYLRYNHA DAEFQERLQE DTSFGGNLGR AVFQAKKRVL |
| | | EPLGLVETPA KTAPGKKRPV DSPDSTSGIG KKGQQPAKKR LNFGQTGDSE |
| | | SVPDPQPIGE PPAGPSGLGS GTMAAGGGAP MADNNEGADG VGNASGNWHC |
| | | DSTWLGDRVI TTSTRTWALP TYNNHLYKQI SSQSAGSTND NVYFGYSTPW |
| | | GYFDFNRFHC HFSPRDWQRL INNNWGFRPK KLNFKLFNIQ VKEVTTNDGV |
| | | TTIANNLTST VQVFSDSEYQ LPYVLGSAHQ GCLPPFPADV FMIPQYGYLT |
| | | LNNGSQSVGR SSFYCLEYFP SQMLRTGNNF TFSYTFEDVP FHSSYAHSQS |
| | | LDRLMNPLID QYLYYLARTQ SNAGGTAGNR ELQFYQGGPT TMAEQAKNWL |
| | | PGPCFRQQRV SKTLDQNNNS NFAWTGATKY HLNXRNSLVN PGVAMATHKD |
| | | DEERFFPSSG VLIFGKTGAA NKTTLENVLM TNEEEIRPTN PVATEEYGIV |
| | | SSNLQAASTA AQTQVVNNQG ALPGMVWQNR DVYLQGPIWA KIPHTDGNFH |
| | | PSPLMGGFGL KHPPPQILIK NTPVPANPPE VFTPAKFASF ITQYSTGQVS |
| | | VEIEWELQKE NSKRWNPEIQ YTSNFDKQTG VDFAVDSQGV YSEP (SEQ ID NO: 84) |
| hu. 12 | — | MAADGYLPDWLEDTLSEGIRQWWKLKPGPPPPKPAERHQDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGHQPARKRLNFGQTGDAD |
| | | SVPDPQPLGQPPAAPTSLGSTTMATGSGAPMADNNEGADGVGNSSGNWHCDSQWLGDRVI |
| | | TTSTRTWALPTYNNHLYKQISSQSGASNDNHYFGYSTPWGYFDFNRFHCHFSPRDWQRLI |
| | | NNNWGFRPKRLNFKLFNIQVKEVTQNDGTTTIANNLTSTVQVFTDSEYQLPYVLGSAHQG |
| | | CLPPFPADVFMVPQYGYLTLNNGSQAVGRPSFYCLEYFPSQMLRTGNNFTFSYTFEDVPF |
| | | HSSYAHSQSLDRLMNPLIDQYLYYLNRTQSNSGTLQQSRLLFSQAGPTSMSLQAKNWLPG |
| | | PCYRQQRLSKQANDNNNSNFPWTAATKYHLNGRDSLVNPGPAMASHKDDEEKFFPMHGTL |
| | | IFGKQGTNANDADLEHVMITDEEEIRTTNPVATEQYGNVSNNLQNSNTGPTTENVNHQGA |
| | | LPGMVWQDRDVYLQGPIWAKIPHTDGHFHPSPLMGGFGLKHPPPQIMIKNTPVPANPPTN |
| | | FSSAKFASFITQYSTGQVSVEIEWELQKENSKRWNPEIQYTSNYNKSVNVDFTVDTNGVY |
| | | SEPRPIGTRYLTRNL (SEQ ID NO: 85) |
| hu.13 | — | MAADGYLPDW LEDTLSEGIR QWWKLKPGPP PPKPAERHKD DSRGLVLPGY |
| | | KYLGPFNGLD KGEPVNEADA AALEHDKAYD RQLDSGDNPY LKYNHADAEF |
| | | QERLKEDTSF GGNLGRAVFQ AKKRVLEPLG LVEEPVKTAP GKKRPVEHSP |
| | | AEPDSSSGTG KAGQQPARKR LNFGQTGDAD SVPDPQPLGQ PPAAPSGLGT |
| | | NTMASGSGAP MADNNEGADG VGNSSGNWHC DSTWMGDRVI TTSTRTWALP |
| | | TYNNHLYKQI SSQSGASNDN HYFGYSTPWG YFDFNRFHCH FSPRDWQRLI |
| | | NNNWGFRPKR LNFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS |
| | | QMLRTGNNFT FSYTFEDVPF HSSYAHSQSL DRLMNPLIDQ YLYYLSRTNT |
| | | PSGTTTQSRL QFSQAGASDI RDQSRNWLPG PCYRQQRVSK TSADNNNSEY |
| | | SWTGATKYHL NGRDSLVNPG PAMASHKDDE EKFFPQSGVL IFGKQGSEKT |
| | | NVDIEKVMIT DEEEIRTTNP VATEQYGSVS TNLQGGNTQA ATADVNTQGV |
| | | LPGMVWQDRD VYLQGPIWAK IPHTDGHFHP SPLMGGFGLK HPPPQILIKN |
| | | TPVPANPSTT FSAAKFASFI TQYSTGQVSV EIEWELQKEN SKRWNPEIQY |
| | | TSNYNKSVNV DFTVDTNGVY SEPRPIGTRY LTRNL (SEQ ID NO: 85) |
| hu.21 | — | MAADGYLPDWLEDTLSEGIRQWWKLKPGPPPPKPAERHKDDSRGLVLPGYKYLGPFNGLDKGEPVNEAD |
| | | AAALEHDKAYDRQLDSGDNPYLKYNHADAEFQERLKEDTSFGGNLGRAVFQAKKRILEPLGLVEEPVKT |
| | | APGKKRPVEHSPAEPDSSSGTGKAGQQPARKRLNFGQTGDADSVPDPRPLGQPPAAPSGLGTNTMASGS |
| | | GAPMADNNEGADGVGNSSGNWHCDSTWMGDRVITTSTRTWALPTYNNHLYKQISSQSGASNDNHYFGYS |
| | | TPWGYFDFNRFHCHFSPRDWQRLINNNWGFRPKRLSFKLFNIQVKEVTQNDGTTTIANNLTSTVQVFTD |
| | | SEYQLPYVLGSAHQGCLPPFPADVFMVPQYGYLTLNNGSQAVGRSSFYCLEYFPSQMLRTGNNFTFSYT |
| | | FEDVPFHSSYAHSQSLDRLMNPLIDQYLYYLSRTNTPSGTTTMSRLQFSQAGASDIRDQSRNWLPGPCY |
| | | RQQRVSKTAADNNNSDYSWTGATKYHLNGRDSLVNPGPAMASHKDDEEKYFPQSGVLIFGKQDSGKTNV |
| | | DIEKVMITDEEEIRTTNPVATEQYGSVSTNLQSGNTQAATSDVNTQGVLPGMVWQDRDVYLQGPIWAKI |
| | | PHTDGHFHPSPLMGGFGLKHPPPQILIKNTPVPANPSTTFSAAKFASFITQYSTGQVSVEIEWELQKEN |
| | | SKRWNPEIQYTSNYNKSVNVDFTVDTNGVYSEPRPIGTRYLTRNL (SEQ ID NO: 74) |
| hu.26 | — | MAADGYLPDW LEDTLSEGIR QWWKLKPGPP PPKPAERHKD DSRGLVLPGY |
| | | KYLGPFNGLD KGEPVNEADA AALEHDKAYD RQLDSGDNPY LKYNHADAEF QERLKEDTSF |
| | | GGNLGRAVFQ AKKRILEPLG LVEEPVKTAP GKKRPVEHSP AEPDSSSGTG KAGQQPARKR |
| | | LNFGQTGDAD SVPDPQPLGQ PPAAPSGLGT NTMASGSGAP MADNNEGADG VGNSSGNWHC |
| | | DSTWMGDRVI TTSTRTWALP TYNNHLYKQI SSQSGASNDN HYFGYSTPWG YFDFNRFHCH |
| | | FSPRDWQRLI NNNWGFRPKR LSFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS QMLRTGNNFT |
| | | FSYTFEDVPF HSSYAHSQSL DRLMNPLIDQ YLYYLSRTNT PSGTTTMSRL QFSQAGASDI |
| | | RDQSRNWLPG PCYRQQRVSK TAADNNNSDY SWTGATKYHL NGRDSLVNPG PAMASHKDDE |
| | | EKYFPQSGVL IFGKQDSGKT NVDIEKVMIT DEEEIRTTNP VATEQYGSVS TNLQSGNTQA |
| | | ATSDVNTQGV LPGMVWQDRD VYLQGPIWAK IPHTDGHFHP SPLMGGFGLK HPPPQILIKN |
| | | TPVPANPSTT FSAAKFASFI TQYSTGQVSV EIEWELQKEN SKRWNPEIQY TSNYNKSVNV |
| | | DFTVDINGVY SEPRPIGTRY LTRNL (SEQ ID NO: 76) |
| hu.53 | - | MAADGYLPDW LEDTLSEGIR QWWKLKPGPP PPKPAERHKD DSRGLVLPGY |
| | | QERLKEDTSF GGNLGRAVFQ AKKRVLEPLG LVEEPVKTAP GKKRPVEHSP |
| | | AEPDSSSGTG KAGQQPARKR LNFGQTGDAD SVPDPQPLRQ PPAAPTSLGS |
| | | TTMATGSGAP MADNNEGADG VGNSSGNWHC DSQWLGDRVI TTSTRTWALP |
| | | TYNNHLYKQI SSQSGASNDN HYFGYSTPWG YFDFNRFHCH FSPRDWQRLI |
| | | NNNWGFRPKR LNFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS |
| | | QMLRTGNNFQ FSYTFEDVPF HSSYAHSQSL DRLMNPLIDQ YLYYLNRTQT |
| | | ASGTQQSRLL FSQAGPTSMS LQAKNWLPGP CYRQQRLSKQ ANDNNNSNFP |
| | | WTGATKYYLN GRDSLVNPGP AMASHKDDEE KFFPMHGTLI FGKEGTNATN |
| | | AELENVMITD EEEIRTTNPV ATEQYGYVSN NLQNSNTAAS TETVNHQGAL |
| | | PGMVWQDRDV YLQGPIWAKI PHTDGHFHPS PLMGGFGLKH PPPQIMIKNT |
| | | PVPANPPTNF SSAKFASFIT QYSTGQVSVE IEWELQKENS KRWNPEIQYT |
| | | SNYNKSVNVD FTVDINGVYS EPRPIGTRYL TRNL (SEQ ID NO: 77) |
| hu.56 | — | MAADGYLPDW LEDTLSEGIR QWWKLKPGPP PPKPAERHKD DSRGLVLPGY |
| | | KYLGPFNGLD KGEPVNEADA AALEHDKAYD RQLDSGDNPY LKYNHADAEF |
| | | QERLKEDTSF GGNLGRAVFQ AKKRVLEPLG LVEEPVKTAP GKKRPVEHSP |
| | | VEPDSSSGTG KAGNQPARKR LNFGQTGDAD SVPDPQPLGQ PPASPSGLGT |
| | | NTMATGSGAP MADNNEGADG VGNSSGNWHC DSTWMGDRVV TTSTRTWALP |
| | | TYNNHLYKQI SSQSGASNDN HYFGYSTPWG YFDFNRFHCH FSPRDWQRLI |
| | | KYLGPFNGLD KGEPVNEADA AALEHDKAYD RQLDSGDNPY LKYNHADAEF |
| | | NNNWGFRPKR LNFKLFNIQV KEVTQNDGTT TIANNLTSTV QVFTDLEYQL |
| | | PYVLGSAHQG CLPPFPADVF MVPQYGYLTL NNGSQAVGRS SFYCLEYFPS |
| | | QMLRTGNNFT FSYTFEDVPF HSSYAHSQSL DRLMNPLIDQ YLYYLSRTNT |
| | | PSGTTTQSRL QFSQAGASDI RDQSRNWLPG PCYRQQRVSK TAADNNNSEY |
| | | SWTGATKYHL NGRDSLVNPG PAMASHKDDE EKFFPQSGVL IFGKQGSEKT |
| | | NVDIEKVMIT DEEEIRTTNP VATEQYGSVS TNLQSGNTQA ATSDVNTQGV |
| | | LPGMVWQDRD VYLQGPIWAK IPHTDGHFHP SPLMGGFGLK HPPPQILIKN |
| | | TPVPANPSTT FSAAKFASFI TQYSTGQVSV EIEWELQKEN SKRWNPEIQY |
| | | TSNYNKSVNV DFTVDTNGVY SEPRPIGTRY LTRNL (SEQ ID NO: 86) |
| Rh.64R | R697W | MAADGYLPDW LEDNLSEGIR EWWDLKPGAP KPKANQQKQD DGRGLVLPGY |
| 1 | | KYLGPFNGLD KGEPVNAADA AALEHDKAYD QQLKAGDNPY LRYNHADAEF |
| | | QERLQEDTSF GGNLGRAVFQ AKKRVLEPLG LVEEGAKTAP GKKRPVEPSP |
| | | QRSPDSSTGI GKKGQQPARK RLNFGQTGDS ESVPDPQPIG EPPAAPSSVG |
| | | SGTMAAGGGA PMADNNEGAD GVGSSSGNWH CDSTWLGDRV ITTSTRTWAL |
| | | PTYNNHLYKQ ISNGTSGGST NDNTYFGYST PWGYFDFNRF HCHFSPRDWQ |
| | | RLINNNWGFR PKRLSFKLFN IQVKEVTQNE GTKTIANNLT STIQVFTDSE |
| | | YQLPYVLGSA HQGCLPPFPA DVFMIPQYGY LTLNNGSQAV GRSSFYCLEY |
| | | FPSQMLRTGN NFSFSYTFED VPFHSSYAHS QSLDRLMNPL IDQYLYYLSR |
| | | TQSTGGTAGT QQLLFSQAGP SNMSAQARNW LPGPCYRQQR VSTTLSQNNN |
| | | SNFAWTGATK YHLNGRDSLV NPGVAMATNK DDEDRFFPSS GILMFGKQGA |
| | | GKDNVDYSNV MLTSEEEIKT TNPVATEQYG VVADNLQQQN TAPIVGAVNS |
| | | QGALPGMVWQ NRDVYLQGPI WAKIPHTDGN FHPSPLMGGF GLKHPPPQIL |
| | | IKNTPVPADP PTAFNQAKLN SFITQYSTGQ VSVEIVWELQ KENSKRWNPE (SEQ ID NO: 90) |
| AAV9. | N272A | MAADGYLPDW LEDNLSEGIR EWWALKPGAP QPKANQQHQD NARGLVLPGY KYLGPGNGLD 60 |
| N272A. | 498_NNN/ | KGEPVNAADA AALEHDKAYD QQLKAGDNPY LKYNHADAEF QERLKEDTSF GGNLGRAVFQ 120 |
| 496N | AAA_500 | AKKRLLEPLG LVEEAAKTAP GKKRPVEQSP QEPDSSAGIG KSGAQPAKKR LNFGQTGDTE 180 |
| NN/A | | SVPDPQPIGE PPAAPSGVGS LTMASGGGAP VADNNEGADG VGSSSGNWHC DSQWLGDRVI 240 |
| AA498 | | TTSTRTWALP TYNNHLYKQI SNSTSGGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 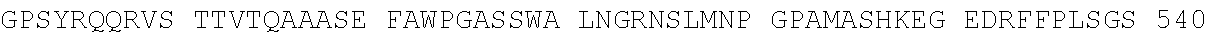 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 91) |
| AAV9. | G266A | MAADGYLPDW LEDNLSEGIR EWWALKPGAP QPKANQQHQD NARGLVLPGY KYLGPGNGLD 60 |
| G266A | 498_NNN/ | KGEPVNAADA AALEHDKAYD QQLKAGDNPY LKYNHADAEF QERLKEDTSF GGNLGRAVFQ 120 |
| 496N | AAA 500 | AKKRLLEPLG LVEEAAKTAP GKKRPVEQSP QEPDSSAGIG KSGAQPAKKR LNFGQTGDTE 180 |
| NN/A | | SVPDPQPIGE PPAAPSGVGS LTMASGGGAP VADNNEGADG VGSSSGNWHC DSQWLGDRVI 240 |
| AA498 | | TTSTRTWALP TYNNHLYKQI SNSTSAGSSN DNAYFGYSTP WGYFDFNRFH CHFSPRDWQR 300 |
| | | LINNNWGFRP KRLNFKLFNI QVKEVTDNNG VKTIANNLTS TVQVFTDSDY QLPYVLGSAH 360 |
| | | EGCLPPFPAD VFMIPQYGYL TLNDGSQAVG RSSFYCLEYF PSQMLRTGNN FQFSYEFENV 420 |
| | | PFHSSYAHSQ SLDRLMNPLI DQYLYYLSKT INGSGQNQQT LKFSVAGPSN MAVQGRNYIP 480 |
| | | 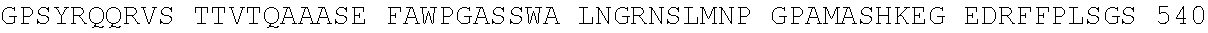 |
| | | LIFGKQGTGR DNVDADKVMI TNEEEIKTTN PVATESYGQV ATNHQSAQAQ AQTGWVQNQG 600 |
| | | ILPGMVWQDR DVYLQGPIWA KIPHTDGNFH PSPLMGGFGM KHPPPQILIK NTPVPADPPT 660 |
| | | AFNKDKLNSF ITQYSTGQVS VEIEWELQKE NSKRWNPEIQ YTSNYYKSNN VEFAVNTEGV 720 |
| | | YSEPRPIGTR YLTRNL (SEQ ID NO: 92) |
| Rh. 46 | | MAADGYLPDWLEDNLSEGIREWWDLKPGAPKPKANQQKQDDGRGLVLPGYKYLGPFNGLD |
| | | KGEPVNAADAAALEHDKAYDQQLKAGDNPYLRYNHADAEFQERLQEDTSFGGNLGRAVFQ |
| | | AKKRVLEPLGLVEEGAKTAPGKKRPVEPSPQRSPDSSTGIGKKGQQPARKRLNFGQTGDS |
| | | ESVPDPQPIGEPPAAPSSVGSGTMAAGGGAPMADNNEGADGVGSSSGNWHCDSTWLGDRV |
| | | ITTSTRTWALPTYNNHLYKQISNGTSGGSTNDNTYFGYSTPWGYFDFNRFHCHFSPRDWQ |
| | | RLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFSFSYTFED |
| | | VPFHSSYAHSQSLDRLMNPLIDQYLYYLSRTQSTGGTAGTQQLLFSQAGPSNMSAQARNW |
| | | LPGPCYRQQRVSTTLSQNNNSNFAWTGATKYHLNGRDSLVNPGVAMATNKDDEDRFFPSS |
| | | GILMFGKQGAGKDNVDYSNVMLTSEEEIKATNPVATEQYGVVADNLQQQNTAPIVGAVNS |
| | | QGALPGMVWQNRDVYLQGPIWAKIPHTDGNFHPSPLMGGFGLKHPPPQILIKNTPVPADP |
| | | PTAFNQAKLNSFITQYSTGQVSVEIEWELQKENSKRWNPEIQYTSNYYKSTNVDFAVNTE |
| | | GVYSEPRPIGTRYLTRNL (SEQ ID NO: 93) |

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A recombinant AAV capsid protein comprising one or more amino acid substitutions relative to the wild type or unengineered capsid protein, in which the rAAV capsid protein is an AAV9 capsid protein (SEQ ID NO:67) with S263G/S269R/A273T substitutions, a G266A substitution, an N272A substitution, a W503R substitution, a Q474A substitution, 496-NNN/AAA-498 substitutions, has an insertion of the peptide TLAAPFK (SEQ ID NO:1) between Q588 and A589, 5268 and 5269, or 5454 and G455, or is an AAV8 capsid (SEQ ID NO:6) with an A269S substitution or 498-NNN/AAA-500 substitutions, or corresponding substitutions or peptide insertions in a capsid protein of another AAV type capsid.

2. The recombinant AAV capsid protein of claim 1 further comprising 498-NNN/AAA-500 amino acid substitutions for an AAV8 capsid protein (SEQ ID NO: 66) or 496-NNN/AAA-498 amino acid substitutions for an AAV9 capsid protein (SEQ ID NO:67), or corresponding substitutions in a capsid protein of another AAV type capsid.

3. The recombinant AAV capsid protein of claim 1 or 2 which is an AAV8.BBB.LD capsid (SEQ ID NO: 27), an AAV9.BBB.LD capsid (SEQ ID NO: 29), an AAV9.496-NNN/AAA-498 capsid (SEQ ID NO: 31), AAV9.496-NNN/AAA-498 capsid (SEQ ID NO: 32), AAV9.W503R capsid (SEQ ID NO: 33), AAV9.Q474A capsid (SEQ ID NO: 34), AAV9.N272A.496-NNN/AAA-498 capsid (SEQ ID NO: 91) or AAV9.N266A.496-NNN/AAA-498 capsid (SEQ ID NO: 92).

4. The recombinant AAV capsid protein of claims 1 to 3 in which the amino acid substitutions or insertions are in an AAV9 capsid, including an AABPHP.eB capsid, protein, or an AAV8 capsid.

5. The recombinant AAV capsid protein of claim 1 or 2 wherein the AAV type capsid is AAV rh.34, AAV4, AAV5, AAV hu.26, AAV rh.31, AAV hu.13, AAV hu.26, AAV hu.56, AAV hu.53, AAV7, AAV rh.10, AAV rh.64.R1, AAV rh.46 or AAV rh.73.

6. The recombinant AAV capsid protein of any of claims 1 to 5, which when incorporated into a rAAV vector, the rAAV vector has increased targeting, transduction or genome integration into CNS cells, relative to a rAAV vector incorporating the corresponding wild type capsid protein without the amino acid substitutions or peptide insertions.

7. The recombinant capsid protein of claim 6, which when incorporated into a rAAV vector, the rAAV vector has decreased targeting, transduction or genome integration into liver cells, relative to a rAAV vector incorporating the corresponding wild type capsid protein without the amino acid substitutions or peptide insertions.

8. The recombinant capsid protein of claim 6 or 7, which when incorporated into a rAAV vector, the rAAV vector has decreased targeting, transduction or genome integration into dorsal root ganglion cells, relative to a rAAV vector incorporating the corresponding capsid protein without the amino acid substitutions or peptide insertions.

9. The recombinant capsid protein of any of the claims 6 to 8, which when incorporated into a rAAV vector, the rAAV vector has decreased targeting, transduction or genome integration into peripheral nerve cells, relative to a rAAV vector incorporating the corresponding capsid protein without the amino acid substitutions or peptide insertions.

10. The recombinant AAV capsid protein of any of claims 1 to 5, which when incorporated into a rAAV vector, the rAAV vector has increased targeting, transduction or genome integration into skeletal and/or cardiac muscle cells, relative to a rAAV vector incorporating the corresponding capsid protein without the amino acid substitutions or peptide insertions.

11. The recombinant capsid protein of claim 10, which when incorporated into a rAAV vector, the rAAV vector has decreased targeting, transduction or genome integration into liver cells, relative to a rAAV vector incorporating the corresponding capsid protein without the amino acid substitutions or peptide insertions.

12. The recombinant capsid protein of claim 10 or 11, which when incorporated into a rAAV vector, the rAAV vector has decreased targeting, transduction or genome integration into CNS cells, relative to a rAAV vector incorporating the corresponding capsid protein without the amino acid substitutions or peptide insertion.

13. The recombinant capsid protein of any of claims 10 to 12, which when incorporated into a rAAV vector, the rAAV vector has decreased targeting, transduction or genome integration into dorsal root ganglion cells, relative to an rAAV vector incorporating the corresponding capsid protein without the amino acid substitutions

14. A nucleic acid comprising a nucleotide sequence encoding the rAAV capsid protein of any of claims 1 to 13, or encoding an amino acid sequence sharing at least 80% identity therewith and retaining the biological activity of the capsid.

15. The nucleic acid of claim 14 encoding the rAAV capsid protein of any of claims 1 to 13.

16. A packaging cell capable of expressing the nucleic acid of claim 14 or 15 to produce AAV vectors comprising the capsid protein encoded by said nucleotide sequence.

17. A rAAV vector comprising the capsid protein of any of claims 1 to 13.

18. The rAAV vector of claim 17 further comprising a transgene encoding a therapeutic protein operably linked to a regulatory sequence for expression in the muscle and/or CNS cells.

19. A pharmaceutical composition comprising the rAAV vector of claim 17 or 18 and a pharmaceutically acceptable carrier.

20. A method of delivering a transgene to a cell, said method comprising contacting said cell with the rAAV vector of claim 17 or 18, wherein said transgene is delivered to said cell.

21. The method of claim 20 in which the cell is a CNS cell, cardiac muscle cell or skeletal muscle cell.

22. A method of delivering a transgene to a target tissue of a subject in need thereof, said method comprising administering to said subject the rAAV vector of claim 17 or 18, wherein the transgene is delivered to said target tissue.

23. The method of claim 22 wherein the transgene is a muscle disease or heart disease therapeutic and said target tissue is cardiac muscle or skeletal muscle.

24. The method of claim 23, wherein the rAAV is administered systemically, including intravenously or intramuscularly.

25. The method of claim 22 wherein the transgene is a CNS disease therapeutic and said target tissue is CNS.

26. The method of claim 25 wherein the rAAV is administered intrathecally or intracerebroventricularly.

27. A pharmaceutical composition for use in delivering a transgene to a cell, said pharmaceutical composition comprising the rAAV vector of claim 17 or 18, wherein said transgene is delivered to said cell.

28. A pharmaceutical composition for use in delivering a transgene encoding a therapeutic protein to a target tissue of a subject in need thereof, said pharmaceutical composition comprising the rAAV vector of claim 17 or 18, wherein the transgene is delivered to said target tissue.

29. The pharmaceutical composition of claim 27 or 28 wherein said therapeutic protein is a muscle disease therapeutic or a heart disease therapeutic and said target tissue is cardiac muscle or skeletal muscle.

30. The pharmaceutical composition of claims 27 to 29 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction in cardiac muscle or skeletal muscle cells compared to a reference AAV capsid.

31. The pharmaceutical composition of claims 27 to 30 wherein the rAAV exhibits at least 50%, 60%, 70%, 80%, 90%, 95% or 99% less transduction in liver compared to the reference AAV capsid.

32. The pharmaceutical composition of claims 27 to 31 wherein the rAAV exhibits at least 50%, 60%, 70%, 80%, 90%, 95% or 99% less transduction in dorsal root ganglion cells compared to the reference AAV capsid.

33. The pharmaceutical composition of claim 27, 28 or 32 wherein said therapeutic protein is a CNS disease therapeutic and said target tissue is CNS.

34. The pharmaceutical composition of claim 27, 28, 32 or 33 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction in CNS cells compared to a reference AAV capsid.

35. The pharmaceutical composition of claims 27, 28, 33 to 34 wherein the rAAV exhibits at least 50%, 60%, 70%, 80%, 90%, 95% or 99% less transduction in liver compared to the reference AAV capsid.

36. The pharmaceutical composition of claims 27, 28, 32 to 35 wherein the rAAV exhibits at least 50%, 60%, 70%, 80%, 90%, 95% or 99% less transduction in dorsal root ganglion cells compared to the reference AAV capsid.

37. The pharmaceutical composition of claims 27 to 36, wherein the AAV reference capsid is AAV8 or AAV9.

38. A method of treating a CNS disorder in a subject in need thereof, said method comprising administering a therapeutically effective amount of pharmaceutical composition of any of claims 27, 28, 32 to 37.

39. A method of treating a muscle disorder in a subject in need thereof, said method comprising administering a therapeutically effective amount of the pharmaceutical composition of any of claims 27-31 and 37.

40. The rAAV vector of claim 18, wherein the transgene is selected from Table 1A or Table 1B.

41. The method of claim 20, or 22-26, wherein the transgene is selected from Table 1A or Table 1B.

42. The pharmaceutical composition for use in delivering a transgene of claim 27 or 28, wherein the transgene is selected from Table 1A or Table 1B.


SEQUENCE LISTING (93 sequences)

SEQ ID NO: 1 - protein, 7 aa, Artificial Sequence
  synthetic construct; capsid peptide
TLAAPFK

SEQ ID NO: 2 - protein, 8 aa, Artificial Sequence
  synthetic construct; capsid peptide
DDDDDDDD

SEQ ID NO: 3 - protein, 7 aa, Artificial Sequence
  synthetic construct; capsid peptide
LSSRLDA

SEQ ID NO: 4 - protein, 9 aa, Artificial Sequence
  synthetic construct; capside peptide
CLSSRLDAC

SEQ ID NO: 5 - protein, 7 aa, Artificial Sequence
  synthetic construct; capside peptide
CLPVASC

SEQ ID NO: 6 - protein, 5 aa, Artificial Sequence
  synthetic construct; capside peptide
LPVAS

SEQ ID NO: 7 - protein, 7 aa, Artificial Sequence
  synthetic construct; capside peptide
ASSLNIA

SEQ ID NO: 8 - protein, 6 aa, Artificial Sequence
  synthetic construct; capside peptide
QAQAQT

SEQ ID NO: 9 - protein, 4 aa, Artificial Sequence
  synthetic construct; capside peptide
STQT

SEQ ID NO: 10 - protein, 7 aa, Artificial Sequence
  synthetic construct; capside peptide
HAIYPRH

SEQ ID NO: 11 - protein, 12 aa, Artificial Sequence
  synthetic construct; capsid peptide
THRPPMWSPVWP

SEQ ID NO: 12 - protein, 7 aa, Artificial Sequence
  synthetic construct; capsid peptide
RTIGPSV

SEQ ID NO: 13 - protein, 9 aa, Artificial Sequence
  synthetic construct; capsid peptide
CRTIGPSVC

SEQ ID NO: 14 - protein, 11 aa, Artificial Sequence
  synthetic construct; capsid peptide
SITLVKSTQTV

SEQ ID NO: 15 - protein, 10 aa, Artificial Sequence
  synthetic construct; capsid peptide
TILSRSTQTG

SEQ ID NO: 16 - protein, 7 aa, Artificial Sequence
  synthetic construct; capsid peptide
QAVRTSL

SEQ ID NO: 17 - protein, 7 aa, Artificial Sequence
  synthetic construct; capsid peptide
QAVRTSH

SEQ ID NO: 18 - protein, 20 aa, Artificial Sequence
  synthetic construct; capsid peptide
VVMVGEKPITITQHSVETEG

SEQ ID NO: 19 - protein, 12 aa, Artificial Sequence
  synthetic construct; capsid peptide
RSSEEDKSTQTT

SEQ ID NO: 20 - protein, 7 aa, Artificial Sequence
  synthetic construct; capsid peptide
TLAVPFK

SEQ ID NO: 21 - protein, 8 aa, Artificial Sequence
  synthetic construct; capsid peptide
DYKDDDDK

SEQ ID NO: 22 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 27 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQAAASNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 28 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQAAASNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 31 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVAGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 744 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDDDDDDDDGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLSSRLDAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 745 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLSSRLDACGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 741 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLPVASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLPVASCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASSLNIAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHAIYPRHGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRTIGPSVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 745 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCRTIGPSVCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 747 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSITLVKSTQTVSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 746 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNTILSRSTQTGSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 753 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTTILSRSTQTGAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 754 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTSITLVKSTQTVAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 35 aa, Artificial Sequence
  synthetic construct; capsid sequence
  misc_feature (14)..(15): Xaa can be any naturally occurring amino acid
  misc_feature (23)..(23): Xaa can be any naturally occurring amino acid
  misc_feature (30)..(30): Xaa can be any naturally occurring amino acid
YYLSRTQNTGGTAXXQTLLFSQXGPSNMSXQAKNW

SEQ ID NO: 49 - protein, 35 aa, Artificial Sequence
  synthetic construct; capsid sequence
YYLNRTQNQSGSAQNKDLLFSRGSPAGMSVQPKNW

SEQ ID NO: 50 - protein, 35 aa, Artificial Sequence; identical to SEQ ID NO: 49
  synthetic construct; capsid sequence

SEQ ID NO: 51 - protein, 35 aa, Artificial Sequence
  synthetic construct; capsid sequence
YYLSRTNTPSGTTTQSRLQFSQAGASDIRDQSRNW

SEQ ID NO: 52 - protein, 36 aa, Artificial Sequence
  synthetic construct; capsid sequence
YYLNRTQGTTSGTTNQSRLLFSQAGPQSMSLQARNW

SEQ ID NO: 53 - protein, 36 aa, Artificial Sequence
  synthetic construct; capsid sequence
WGLQSTTTGTTLNAGTATTNFTKLRPTNFSNFKKNW

SEQ ID NO: 54 - protein, 29 aa, Artificial Sequence
  synthetic construct; capsid sequence
YRFVSTNNTGGVQFNKNLAGRYANTYKNW

SEQ ID NO: 55 - protein, 36 aa, Artificial Sequence
  synthetic construct; capsid sequence
YYLARTQSNPGGTAGNRELQFYQGGPSTMAEQAKNW

SEQ ID NO: 56 - protein, 35 aa, Artificial Sequence
  synthetic construct; capsid sequence
YYLSRTQTTGGTANTQTLGFSQGGPNTMANQAKNW

SEQ ID NO: 57 - protein, 34 aa, Artificial Sequence
  synthetic construct; capsid sequence
YYLSKTINGSGQNQQTLKFSVAGPSNMAVQGRNY

SEQ ID NO: 58 - protein, 35 aa, Artificial Sequence
  synthetic construct; capsid sequence
YYLSRTQSTGGTAGTQQLLFSQAGPNNMSAQAKNW

SEQ ID NO: 59 - protein, 736 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 60 - protein, 735 aa, Artificial Sequence [sequence omitted]

SEQ ID NO: 61 - protein, 736 aa, Artificial Sequence
  synthetic construct; capsid sequence
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 62 - protein, 734 aa, Artificial Sequence
  synthetic construct; capsid sequence
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 63 - protein, 724 aa, Artificial Sequence
  synthetic construct; capsid sequence
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 64 - protein, 736 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 65 - protein, 737 aa, Artificial Sequence
  synthetic construct; capsid sequence
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 66 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 67 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 68 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 70 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 71 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 72 - protein, 738 aa, Artificial Sequence
  synthetic construct; capsid sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 73 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 74 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 75 - protein, 735 aa, Artificial Sequence
  synthetic construct; capsid sequence
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 76 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 77 - protein, 734 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 78 - protein, 736 aa, Artificial Sequence; identical to SEQ ID NO: 61
  synthetic construct; capsid sequence

SEQ ID NO: 79 - protein, 734 aa, Artificial Sequence; identical to SEQ ID NO: 62
  synthetic construct; capsid sequence

SEQ ID NO: 80 - protein, 738 aa, Artificial Sequence
  synthetic construct; capsid sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 81 - protein, 6 aa, Artificial Sequence
  synthetic construct; capsid sequence
HAIYPR

SEQ ID NO: 82 - protein, 733 aa, Artificial Sequence
  synthetic construct; capsid sequence
MAADGYLPDWLEDNLSEGIREWWDLKPGAPKPKANQQKQDDGRGLVLPGYEYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFETAYNFEKVPFHSMYAHSQSLDGLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPAYPATTFTAARVDSFITQYSTGQVAVQIEWEIEKERSKRWNPEVQFTSNCGNQSSMLWAPDTTGKYTEPRVIGSRYLTNHL

SEQ ID NO: 83 - protein, 736 aa, Artificial Sequence
  synthetic construct; capsid sequence
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 84 - protein, 644 aa, Artificial Sequence
  synthetic construct; capsid sequence
  misc_feature (434)..(434): Xaa can be any naturally occurring amino acid
KAYDQQLKAGDNPYLRYNHADAEFQERLQEDTSFGGNLGRAVFQAKKRVLEPLGLVETPAKTAPGKKRPVDSPDSTSGIGKKGQQPAKKRLNFGQTGDSESVPDPQPIGEPPAGPSGLGSGTMAAGGGAPMADNNEGADGVGNASGNWHCDSTWLGDRVITTSTRTWALPTYNNHLYKQISSQSAGSTNDNVYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNAGGTAGNRELQFYQGGPTTMAEQAKNWLPGPCFRQQRVSKTLDQNNNSNFAWTGATKYHLNXRNSLVNPGVAMATHKDDEERFFPSSGVLIFGKTGAANKTTLENVLMTNEEEIRPTNPVATEEYGIVSSNLQAASTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKQTGVDFAVDSQGVYSEP

SEQ ID NO: 85 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 86 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGNQPARKRLNFGQTGDADSVPDPQPLGQPPASPSGLGTNTMATGSGAPMADNNEGADGVGNSSGNWHCDSTWMGDRVVTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDLEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTAADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 87 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAAPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 88 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSTLAAPFKSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 89 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTLAAPFKGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 90 - protein, 700 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPE

SEQ ID NO: 91 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDAAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 92 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSAGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 93 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL